(19) United States
(12) Patent Application Publication
(10) Pub. No.: US 2021/0371855 A1
(43) Pub. Date: Dec. 2, 2021

(54) METHODS OF INHIBITING PROLIFERATIVE CELLS

(71) Applicant: IDEAYA BIOSCIENCES, INC., South San Francisco, CA (US)

(72) Inventors: Lisa BELMONT, South San Francisco, CA (US); Jeff HAGER, South San Francisco, CA (US); Yujiro HATA, South San Francisco, CA (US); Lorn KATEGAYA, South San Francisco, CA (US)

(21) Appl. No.: 17/252,430

(22) PCT Filed: Jun. 17, 2019
(86) PCT No.: PCT/US2019/037559

§ 371 Date: Dec. 15, 2020

Related U.S. Application Data
(60) Provisional application No. 62/685,866, filed on Jun. 15, 2018.

(51) Int. Cl. C12N 15/11; A61P 35/00; A61K 31/4015; A61K 47/68; C12N 15/113; C12N 9/22; A61K 38/46; A61K 31/7088; C12N 15/86; C12N 9/10; A61K 38/45; C12Q 1/533; A61K 45/06

(52) U.S. Cl. CPC C12N 15/11; C12N 2310/531; A61K 31/4015; A61K 47/6803; C12N 15/1137; C12N 9/22; A61K 38/465; A61K 31/7088; C12N 15/86; C12N 9/104; A61K 38/45; C12Y 203/02; C12Q 1/533; A61K 45/06; C12N 2310/20; C12N 2800/80; C12N 2750/14143; G01N 2333/99; C12N 2310/14; C12N 2320/12; C12N 2310/122; A61P 35/00

(57) ABSTRACT

The present invention provides methods of negatively modulating the Werner protein (WRN) to inhibit proliferative cells characterized by high microsatellite instability (MSI-H), for example to treat proliferative diseases (such as cancer) characterized by high MSI (MSI-H). Further provided are compositions used in such methods.

### CROSS-REFERENCES TO RELATED APPLICATIONS

This application claims the benefit of priority under 35 U.S.C § 119(e) to U.S. Provisional Application Ser. No. 62/685,866, filed Jun. 15, 2018, the disclosure of which is incorporated herein by reference in its entirety.

### FIELD

The present disclosure relates to methods of negatively modulating the Werner protein (WRN) to inhibit proliferative cells characterized by high microsatellite instability (MSI-H), for example to treat proliferative diseases (such as cancer) characterized by high MSI (MSI-H). Further provided are compositions used in such methods.

### BACKGROUND OF THE INVENTION

Cancer is a leading cause of death throughout the world. A limitation of prevailing therapeutic approaches, e.g. chemotherapy is that their cytotoxic effects are not restricted to cancer cells and adverse side effects can occur within normal tissues. Consequently, novel strategies are urgently needed to better target cancer cells.

Synthetic lethality arises when a combination of deficiencies in the expression of two or more genes or corresponding loss of function of related gene product proteins (e.g., resulting from one or more chromosomal mutations) leads to cell death, whereas a singular deficiency/loss of function does not. For example, one of the genes (or gene products) can be involved in cell proliferation, whereas the other of the genes (or gene products) can be a non-essential gene. The concept of synthetic lethality originates from studies in drosophila model systems in which a combination of mutations in two or more separate genes leads to cell death (in contrast to viability, or even cell proliferation, which occurs when only one of the genes is mutated or deleted). More recently, studies have explored maladaptive genetic changes in cancer cells that render them vulnerable to synthetic-lethality approaches. These tumor-specific genetic defects can create a vulnerability, which enable the use of targeted agents that are synthetically lethal to such tumor-specific genomic defect and induce the death of tumor cells while sparing normal cells.

Disruptions in DNA repair pathways predispose cells to accumulating DNA damage. Various types of tumors are known to accumulate progressively more mutations in DNA repair proteins as cancers progress. Therefore, pathways involved in DNA repair mechanisms can be targeted by cytotoxic treatments based on synthetic lethality, turning dysregulated repair processes against themselves to induce tumor death.

Identifying synthetic lethal (SL) interactions that are relevant in cancers is an area of focus for biological discovery efforts. In a yeast screen looking to uncover SL interactions between tumor suppressor genes and drug targets, Sgs1, a RECQ helicase, was found to be SL with several genes in the screen. In the same study using human cells, Bloom (BLM), one of five human RECQ helicases, was SL with Check point kinase 1 and 2 (Srivas R, et al., Mol Cell 2016; 63(3):514-25). During DNA damage repair (DDR), BLM participates in homologous recombination (HR). RECQ helicases are 3′ to 5′ DNA unwinding DNA-dependent ATPases. Three RECQ helicases, BLM, Werner (WRN) and RECQL4, cause human syndromes that overlap, but are also distinct symptomatically, when their expression is altered or lost (de Renty C, Ellis N A. Ageing Res Rev 2017; 33:36-51). This suggests that they may have overlapping and distinct functions based on when and where they are expressed in cells, their protein-protein interactions, post-translational modifications, and the like.

Another study (DRIVE) using approximately 400 cell lines includes data from which it can be shown that WRN is not broadly essential but that microsatellite instability (MSI) cell lines from large intestine, endometrial and stomach tissues of origin are sensitive to WRN shRNAs (McDonald E R, 3rd, de Weck A, Schlabach M R, Billy E, Mavrakis K J, Hoffman G R, et al., Cell 2017; 170(3):577-92). The DepMap study, which derives in part from the DRIVE data, also found a pattern of WRN essentiality in MSI cell lines (Tsherniak A, Vazquez F, Montgomery P G, Weir B A, Kryukov G, Cowley G S, et al., Cell 2017; 170(3):564-76). None of the other human RECQ helicases tested in the study showed this MSI SL interaction.

In the DRIVE dataset, two MSI cell lines derived from the same patient (DLD-1 and HCT-15), were not sensitive to WRN knockdown. DLD-1 cell lines do not express MSH6. Additionally, they have one WT copy and normal expression of MRE11, a protein involved in DDR pathways, such as HR and non-canonical NHEJ, that is often reduced in MSI cell lines because of 1 or 2 deleted thymidines in intron 4 (Koh K H, Kang H J, Li L S, Kim N G, You K T, Yang E, et al., Lab Invest 2005; 85(9):1130-8). Homozygous thymidine deletions lead to a reduction of MRE11 expression.

There is a need for identifying new SL interactions, as well as for further characterizing existing SL interactions, that would allow for new and useful targets for various indications characterized by diseased or otherwise aberrant cells.

### SUMMARY

Several aspects described herein relate to methods of decreasing proliferation of proliferative cells characterized by high MSI, use of such methods in treating proliferative diseases, and compositions used in such methods.

In one aspect, provided herein is a pharmaceutical agent for use in a method for treating a proliferative disease in an individual in need thereof, the proliferative disease being characterized by proliferating cells having a high microsatellite instability (MSI-H), said method comprising administering to the individual said pharmaceutical agent, wherein said pharmaceutical agent is effective for decreasing the helicase activity of WRN in the proliferative cells. In some embodiments, the pharmaceutical agent is an inhibitor of WRN. In some embodiments, the inhibitor of WRN is a small molecule inhibitor.

In some embodiments, according to any of the embodiments described above, the small molecule inhibitor has the formula:

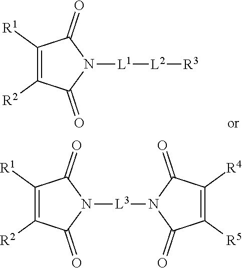

or a pharmaceutically acceptable salt thereof, wherein

L₁ is C₁₋₄ alkylene;
L² is O, S, OC(O), OSO₂, OC(O)O, or OC(O)NH;
L³ is C₁₋₈ alkylene;
R¹, R², R⁴, and R⁵ are each independently H, halogen, C₁₋₄ alkyl, or C₁₋₄ haloalkyl; and
R³ is H, C₁₋₆ alkyl, C₂₋₆ alkenyl, C₂₋₆ alkynyl, C₁₋₆ hydroxyalkyl, C₁₋₆ haloalkyl, C₆₋₁₂ aryl, or C₆₋₁₂ aryl-C₁₋₄ alkyl, each of which is optionally substituted with halogen, C₁₋₄ alkyl, or C₁₋₄ haloalkyl.

In some embodiments, according to any of the embodiments described above, the inhibitor of WRN is an ADC comprising an antibody conjugated to a WRN inhibitor.

In some embodiments, according to any of the embodiments described above, the pharmaceutical agent is an inhibitory nucleic acid targeting WRN mRNA, or a nucleic acid encoding the inhibitory nucleic acid. In some embodiments, the inhibitory nucleic acid comprises a short interfering RNA (siRNA), a microRNA (miRNA), or an antisense oligonucleotide.

In some embodiments, according to any of the embodiments described above, the pharmaceutical agent is a nuclease capable of modifying the genomes of the proliferative cells such that the helicase activity of WRN in the proliferative cells is decreased, or a nucleic acid encoding the nuclease. In some embodiments, the nuclease is a transcription activator-like effector nuclease (TALEN) or zinc-finger nuclease (ZFN) targeting a genomic sequence within or near an endogenous WRN gene locus. In some

In some embodiments, according to any of the embodiments described above, the method comprises administering to the individual a) a gRNA comprising a spacer sequence that is complementary to a genomic sequence within or near an endogenous WRN gene locus, or a nucleic acid encoding the gRNA; and b) an RNA-guided endonuclease (RGEN), or a nucleic acid encoding the RGEN. In some embodiments, the nucleic acid encoding the gRNA is contained in an Adeno Associated Virus (AAV) vector and/or the nucleic acid encoding the RGEN is contained in an AAV vector. In some embodiments, the spacer sequence is complementary to a genomic sequence within a coding region of the endogenous WRN gene. In some embodiments, the genomes of the proliferative cells are modified by non-homologous end joining (NHEJ).

In some embodiments, according to any of the embodiments described above, the method further comprises administering to the individual a donor template comprising a donor nucleic acid, wherein the donor template is configured such that the donor nucleic acid is capable of being inserted into the WRN gene locus by homologous recombination. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession.

In some embodiments, according to any of the embodiments described above, the donor nucleic acid encodes a mutation in the WRN helicase domain that decreases WRN helicase activity.

In some embodiments, according to any of the embodiments described above, the donor template is contained in an AAV vector.

In some embodiments, according to any of the embodiments described above, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof. In some embodiments, the RGEN is Cas9.

In some embodiments, according to any of the embodiments described above, the nucleic acid encoding the RGEN is a ribonucleic acid (RNA) sequence. In some embodiments, the RNA sequence encoding the RGEN is linked to the gRNA via a covalent bond.

In some embodiments, according to any of the embodiments described above, the nucleic acid encoding the RGEN is a deoxyribonucleic acid (DNA) sequence.

In some embodiments, according to any of the embodiments described above, the nucleic acid encoding the RGEN is formulated in a liposome or lipid nanoparticle. In some embodiments, the liposome or lipid nanoparticle encapsulates the gRNA.

In some embodiments, according to any of the embodiments described above, the RGEN is pre-complexed with the gRNA, forming a Ribonucleoprotein (RNP) complex.

In some embodiments, according to any of the embodiments described above, the pharmaceutical agent is a nucleic acid construct comprising a donor nucleic acid, wherein the nucleic acid construct is configured such that insertion of the donor nucleic acid into the genomes of the proliferative cells by homologous recombination decreases the helicase activity of WRN in the proliferative cells. In some embodiments, the nucleic acid construct is an AAV vector. In some embodiments, the AAV vector comprises two homology arms having sequences identical or substantially homologous to regions of the endogenous WRN gene. In some embodiments, the AAV vector is an AAV clade F vector.

In some embodiments, according to any of the embodiments described above, the pharmaceutical agent is a proteolysis targeting chimera (PROTAC) that targets WRN for ubiquitination and proteolytic degradation. In some embodiments, the PROTAC comprises an E3 ubiquitin ligase ligand coupled via a linker to a WRN ligand.

In some embodiments, according to any of the embodiments described above, the proliferative cells comprise one or more MSI-H markers selected from the group consisting of BAT25, BAT26, D2S123, D5S346, and D17S250. In some embodiments, the method further comprises determining the presence of the one or more MSI-H markers in a population of proliferative cells from the individual to identify the presence of MSI-H in the proliferative cells. In some embodiments, the step of determining the presence of the one or more MSI-H markers is carried out prior to administering the pharmaceutical agent.

In some embodiments, according to any of the embodiments described above, the proliferative cells comprises a mutation that impairs DNA mismatch repair. In some embodiments, the proliferative cell comprises a mutation in a MutS homolog and/or a mutation in a MutL homolog. In some embodiments, the MutS homolog is selected from the group consisting of MSH2, MSH3, and MSH6, and the MutL homolog is selected from the group consisting of MLH1, MLH3, PMS1, and PMS2. In some embodiments, the proliferative cell comprises a mutation in MLH1, MSH2, and/or PMS2. In some embodiments, the method further comprises determining the presence of the mutation in a population of proliferative cells from the individual to identify the presence of the mutation in the proliferative cells. In some embodiments, the step of determining the presence of the mutation is carried out prior to administering the pharmaceutical agent.

In some embodiments, according to any of the embodiments described above, the proliferative cell comprises one or more markers of DNA damage. In some embodiments, the one or more markers of DNA damage are selected from the group consisting of high p21 expression and high γH2AX expression. In some embodiments, the method further comprises determining the presence of the one or more markers of DNA damage in a population of proliferative cells from the individual to identify the presence of the one or more markers of DNA damage in the proliferative cells. In some embodiments, the step of determining the presence of the one or more markers of DNA damage is carried out prior to administering the pharmaceutical agent.

In some embodiments, according to any of the embodiments described above, the amount of proliferative cells in the individual is decreased as compared to a corresponding individual that does not receive administration of the pharmaceutical agent.

In some embodiments, according to any of the embodiments described above, the rate of proliferation of the proliferative cells is decreased as compared to a corresponding individual that does not receive administration of the pharmaceutical agent.

In some embodiments, according to any of the embodiments described above, at least some of the proliferative cells are induced to undergo cell cycle arrest.

In some embodiments, according to any of the embodiments described above, at least some of the proliferative cells are induced to undergo apoptosis.

In some embodiments, according to any of the embodiments described above, the proliferative disease is a cancer. In some embodiments, the cancer is selected from the group consisting of colon cancer, gastric cancer, endometrium cancer, ovarian cancer, hepatobiliary tract cancer, urinary tract cancer, brain cancer, and skin cancer.

In some embodiments, according to any of the embodiments described above, the method further comprises administering to the individual a conventional therapy for the proliferative disease. In some embodiments, the method comprises administering to the individual an anti-PD-1 therapy.

In some embodiments, according to any of the embodiments described above, the individual is (a) a mammal; (b) a human; or (c) a veterinary animal.

In another aspect, provided herein is a pharmaceutical agent effective for decreasing WRN helicase activity for use in a method of treating an individual with a proliferative disease, the method comprising determining the presence of a high microsatellite instability (MSI-H), or a marker associated with an MSI-H, in a population of proliferative cells from the individual, determining a likelihood that the individual will respond to a therapy comprising administering to the individual said pharmaceutical agent based on the determination of the presence of MSI-H, or a marker associated with MSI-H, in the population of proliferative cells, and administering to the individual said pharmaceutical agent if the individual is predicted to respond to the therapy. In some embodiments, the determination of the presence of MSI-H in the population of proliferative cells comprises determining the presence of one or more MSI markers selected from the group consisting of BAT25, BAT26, D2S123, D5S346, and D17S250. In some embodiments, the individual is predicted to respond to the therapy if the amount of cells in the population of proliferative cells determined to have at least one of the MSI-H markers is above a pre-determined threshold for the proliferative disease. In some embodiments, the individual is predicted not to respond to the therapy if (a) the amount of cells in the population of proliferative cells determined to have at least one of the MSI-H markers is below a pre-determined threshold for the proliferative disease; or (b) the population of proliferative cells is determined to have none of the MSI-H markers.

In some embodiments, according to any of the embodiments described above, the determination of the presence of a marker associated with MSI-H in the population of proliferative cells comprises determining the presence of a mutation that impairs DNA mismatch repair. In some embodiments, the mutation comprises a mutation in a MutS homolog and/or a mutation in a MutL homolog. In some embodiments, the MutS homolog is selected from the group consisting of MSH2, MSH3, and MSH6, and the MutL homolog is selected from the group consisting of MLH1, MLH3, PMS1, and PMS2. In some embodiments, the mutation comprises a mutation in MLH1, MSH2, and/or PMS2.

In some embodiments, according to any of the embodiments described above, the determination of the presence of a marker associated with MSI-H in the population of proliferative cells comprises determining the presence of one or more markers of DNA damage. In some embodiments, the one or more markers of DNA damage are selected from the group consisting of high p21 expression and high γH2AX expression.

In some embodiments, according to any of the embodiments described above, the individual is predicted to respond to the therapy if the amount of cells in the population of proliferative cells determined to have (i) at least one mutation that impairs DNA mismatch repair and/or (ii) at least one marker of DNA damage is above a pre-determined threshold for the proliferative disease. In some embodiments, the at least one mutation that impairs DNA mismatch repair comprises a mutation in MLH1, MSH2, and/or PMS2, and the at least one marker of DNA damage comprises high p21 expression and/or high γH2AX expression.

In some embodiments, according to any of the embodiments described above, the individual is predicted not to respond to the therapy if (a) the amount of cells in the population of proliferative cells determined to have (i) at least one mutation that impairs DNA mismatch repair and/or (ii) at least one marker of DNA damage is below a pre-determined threshold for the proliferative disease; or (b) the population of proliferative cells is determined to have no mutations that impair DNA mismatch repair and no DNA damage markers.

In another aspect, provided herein is an in vitro method for detecting a high microsatellite instability (MSI-H) and the helicase activity of WRN in an individual diagnosed with or thought to have a proliferative disease, the method comprising: (a) contacting a biological sample from the individual with one or more reagents for detecting the presence of an MSI and the helicase activity of WRN; and (b) detecting (i) the presence of an MSI-H; and (ii) the helicase activity of WRN. In some embodiments, the reagent for detecting the presence of an MSI-H in a biological sample comprises a reagent for detecting the presence of one or more MSI markers selected from the group consisting of BAT25, BAT26, D2S123, D5S346, and D17S250.

In another aspect, provided herein is an in vitro method for detecting a marker associated with a high microsatellite instability (MSI-H) and the helicase activity of WRN in an individual diagnosed with or thought to have a proliferative disease, the method comprising: (a) contacting a biological sample from the individual with one or more reagents for detecting the presence of a marker associated with an MSI-H and the helicase activity of WRN helicase; and (b) detecting (i) the presence of the marker associated with an MSI-H; and (ii) the helicase activity of WRN helicase. In some embodiments, the reagent for detecting the presence of a marker associated with an MSI-H in a biological sample comprises a reagent for detecting the presence of (i) one or more mutations that impair DNA mismatch repair and/or (ii) one or more markers of DNA damage. In some embodiments, the one or more mutations that impair DNA mismatch repair comprise a mutation in a MutS homolog and/or a mutation in a MutL homolog. In some embodiments, the MutS homolog is selected from the group consisting of MSH2, MSH3, and MSH6, and the MutL homolog is selected from the group consisting of MLH1, MLH3, PMS1, and PMS2. In some embodiments, the one or more mutations comprise a mutation in MLH1, MSH2, and/or PMS2. In some embodiments, the one or more markers of DNA damage are selected from the group consisting of high p21 expression and high γH2AX expression.

In some embodiments, according to any of the embodiments described above, the proliferative disease is a cancer. In some embodiments, the cancer is selected from the group consisting of colon cancer, gastric cancer, endometrium cancer, ovarian cancer, hepatobiliary tract cancer, urinary tract cancer, brain cancer, and skin cancer.

In some embodiments, according to any of the embodiments described above, the individual is (a) a mammal; (b) a human; or (c) a veterinary animal.

In another aspect, provided herein is a composition comprising (a) a gRNA comprising a spacer sequence complementary to a genomic sequence within or near an endogenous WRN gene locus, or a nucleic acid encoding the gRNA; and b) an RNA-guided endonuclease (RGEN), or a nucleic acid encoding the RGEN, wherein the components of the composition are configured such that delivery of the composition into a cell is capable of decreasing the helicase activity of WRN in the cell. In some embodiments, the nucleic acid encoding the gRNA is contained in an Adeno Associated Virus (AAV) vector and/or the nucleic acid encoding the RGEN is contained in an AAV vector. In some embodiments, the spacer sequence is complementary to a genomic sequence within a coding region of the endogenous WRN gene.

In some embodiments, according to any of the embodiments described above, the composition further comprises a donor template comprising a donor nucleic acid, wherein the donor template is configured such that the donor nucleic acid is capable of being inserted into the WRN gene locus by homologous recombination. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in the WRN helicase domain that decreases WRN helicase activity. In some embodiments, the donor template is contained in an AAV vector.

In some embodiments, according to any of the embodiments described above, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof. In some embodiments, the RGEN is Cas9.

In some embodiments, according to any of the embodiments described above, the nucleic acid encoding the RGEN is a ribonucleic acid (RNA) sequence. In some embodiments, the RNA sequence encoding the RGEN is linked to the gRNA via a covalent bond.

In some embodiments, according to any of the embodiments described above, the nucleic acid encoding the RGEN is a deoxyribonucleic acid (DNA) sequence.

In some embodiments, according to any of the embodiments described above, the nucleic acid encoding the RGEN is formulated in a liposome or lipid nanoparticle. In some embodiments, the liposome or lipid nanoparticle encapsulates the gRNA.

In some embodiments, according to any of the embodiments described above, the RGEN is pre-complexed with the gRNA, forming a Ribonucleoprotein (RNP) complex.

In another aspect, provided here is a composition comprising a nucleic acid construct comprising a donor nucleic acid, wherein the nucleic acid construct is configured such that insertion of the donor nucleic acid into the genome of a proliferative cell by homologous recombination decreases the helicase activity of WRN in the proliferative cell. In some embodiments, the nucleic acid construct is an AAV vector. In some embodiments, the AAV vector comprises two homology arms having sequences identical or substantially homologous to regions of the endogenous WRN gene. In some embodiments, the AAV vector is an AAV clade F vector.

In another aspect, provided here is a method for decreasing proliferation in a proliferative cell having a high microsatellite instability (MSI-H), comprising decreasing the helicase activity of Werner syndrome ATP-dependent helicase (WRN) in the proliferative cell. In some embodiments, the method comprises delivering into the proliferative cell an inhibitor of WRN. In some embodiments, the inhibitor of WRN is a small molecule inhibitor. In some embodiments, the small molecule inhibitor has the formula:

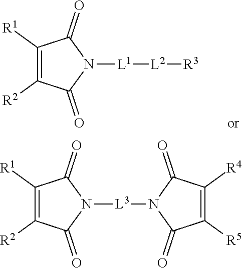

or a pharmaceutically acceptable salt thereof,
wherein
L¹ is C₁₋₄ alkylene;
L² is O, S, OC(O), OSO₂, OC(O)O, or OC(O)NH;
L³ is C₁₋₈ alkylene;
R¹, R², R⁴ and R⁵ are each independently H, halogen, C₁₋₄ alkyl, or C₁₋₄ haloalkyl; and
R³ is H, C₁₋₆ alkyl, C₂₋₆ alkenyl, C₂₋₆ alkynyl, C₁₋₆ hydroxyalkyl, C₁₋₆ haloalkyl, C₆₋₁₂ aryl, or C₆₋₁₂ aryl-C₁₋₄ alkyl, each of which is optionally substituted with halogen, C₁₋₄ alkyl, or C₁₋₄ haloalkyl.

In some embodiments, according to any of the embodiments described above, the inhibitor of WRN comprises an antibody drug conjugate (ADC) comprising an antibody conjugated to the WRN inhibitor.

In some embodiments, according to any of the embodiments described above, the method comprises delivering into the proliferative cell an inhibitory nucleic acid targeting WRN mRNA, or a nucleic acid encoding the inhibitory nucleic acid. In some embodiments, the inhibitory nucleic acid comprises a short interfering RNA (siRNA), a microRNA (miRNA), or an antisense oligonucleotide.

In some embodiments, according to any of the embodiments described above, the method comprises delivering into the proliferative cell a nuclease capable of modifying the genome of the proliferative cell such that the helicase activity of WRN in the proliferative cell is decreased, or a nucleic acid encoding the nuclease. In some embodiments, the nuclease is a transcription activator-like effector nuclease (TALEN) or zinc-finger nucleases (ZFN) targeting a genomic sequence within or near an endogenous WRN gene locus.

In some embodiments, according to any of the embodiments described above, the method comprises delivering into the proliferative cell a) a gRNA comprising a spacer sequence that is complementary to a genomic sequence within or near an endogenous WRN gene locus, or a nucleic acid encoding the gRNA; and b) an RNA-guided endonuclease (RGEN), or a nucleic acid encoding the RGEN. In some embodiments, the nucleic acid encoding the gRNA is contained in an Adeno Associated Virus (AAV) vector and/or the nucleic acid encoding the RGEN is contained in an AAV vector. In some embodiments, the spacer sequence is complementary to a genomic sequence within a coding region of the endogenous WRN gene. In some embodiments, the genome of the proliferative cell is modified by non-homologous end joining (NHEJ).

In some embodiments, according to any of the embodiments described above, the method further comprises delivering into the proliferative cell a donor template comprising a donor nucleic acid, wherein the donor template is configured such that the donor nucleic acid is capable of being inserted into the WRN gene locus by homologous recombination. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in the WRN helicase domain that decreases WRN helicase activity. In some embodiments, the donor template is contained in an AAV vector.

In some embodiments, according to any of the embodiments described above, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof. In some embodiments, the RGEN is Cas9.

In some embodiments, according to any of the embodiments described above, the nucleic acid encoding the RGEN is a ribonucleic acid (RNA) sequence. In some embodiments, the RNA sequence encoding the RGEN is linked to the gRNA via a covalent bond.

In some embodiments, according to any of the embodiments described above, the nucleic acid encoding the RGEN is a deoxyribonucleic acid (DNA) sequence.

In some embodiments, according to any of the embodiments described above, the nucleic acid encoding the RGEN is formulated in a liposome or lipid nanoparticle. In some embodiments, the liposome or lipid nanoparticle encapsulates the gRNA.

In some embodiments, according to any of the embodiments described above, the RGEN is pre-complexed with the gRNA, forming a Ribonucleoprotein (RNP) complex.

In some embodiments, according to any of the embodiments described above, the method comprises delivering into the proliferative cell a nucleic acid construct comprising a donor nucleic acid, wherein the nucleic acid construct is configured such that insertion of the donor nucleic acid into the genome of the proliferative cell by homologous recombination decreases the helicase activity of WRN in the proliferative cell. In some embodiments, the nucleic acid construct is an AAV vector. In some embodiments, the AAV vector comprises two homology arms having sequences identical or substantially homologous to regions of the endogenous WRN gene. In some embodiments, the AAV vector is an AAV clade F vector.

In some embodiments, according to any of the embodiments described above, the method comprises delivering into the proliferative cell a PROTAC that targets WRN for ubiquitination and proteolytic degradation. In some embodiments, the PROTAC comprises an E3 ubiquitin ligase ligand coupled via a linker to a WRN ligand.

In some embodiments, according to any of the embodiments described above, the proliferative cell comprises one or more MSI markers selected from the group consisting of BAT25, BAT26, D2S123, D5S346, and D17S250.

In some embodiments, according to any of the embodiments described above, the proliferative cell comprises a mutation that impairs DNA mismatch repair. In some embodiments, the proliferative cell comprises a mutation in a MutS homolog and/or a mutation in a MutL homolog. In some embodiments, the MutS homolog is selected from the group consisting of MSH2, MSH3, and MSH6, and the MutL homolog is selected from the group consisting of MLH1, MLH3, PMS1, and PMS2. In some embodiments, the proliferative cell comprises a mutation in MLH1, MSH2, and/or PMS2.

In some embodiments, according to any of the embodiments described above, the proliferative cell comprises one or more markers of DNA damage. In some embodiments, the one or more markers of DNA damage are selected from the group consisting of high p21 expression and high γH2AX expression.

In some embodiments, according to any of the embodiments described above, decreasing proliferation in the proliferative cell comprises inducing cell cycle arrest in the proliferative cell.

In some embodiments, according to any of the embodiments described above, decreasing proliferation in the proliferative cell comprises inducing apoptosis in the proliferative cell.

In some embodiments, according to any of the embodiments described above, the method is carried out in vivo.

In some embodiments, according to any of the embodiments described above, the method is carried out ex vivo.

In some embodiments, according to any of the embodiments described above, the method is carried out in vitro.

In some embodiments, according to any of the embodiments described above, the proliferative cell is a cancer cell. In some embodiments, the cancer is selected from the group consisting of colon cancer, gastric cancer, endometrium cancer, ovarian cancer, hepatobiliary tract cancer, urinary tract cancer, brain cancer, and skin cancer.

In some embodiments, according to any of the embodiments described above, the proliferative cell is (a) a mammalian cell; (b) a human cell; or (c) a veterinary animal cell.

### BRIEF DESCRIPTION OF THE DRAWINGS

FIG. 1 depicts a series of graphs showing change in WRN, BLM, and MLH1 transcript expression following siRNA treatment.

FIG. 2 is a series of graphs depicting MSI cell lines are sensitive to WRN knockdown while MSS cell lines are not.

FIG. 3A depicts a series of fluorescent micrographs showing WRN knockdown increases γH2AX and p21 levels in MSI cells. FIG. 3B depicts a series of graphs quantifying the percentage of γH2AX and p21 positive cells. FIG. 3C depicts a series of graphs showing that WRN knockdown alters the cell cycle of MSI cells.

FIG. 4A depicts a schematic showing siRNAs directed to the 5′UTR or to exon 8 of WRN mRNA. FIG. 4B depicts the result of a western blot using the 5′UTR siRNA.

FIG. 4C is a series of graphs depicting the WRN helicase domain rescues the WRN knockdown loss of proliferation phenotype in MSI cells.

FIG. 5 depicts, without being bound by theory, a proposed model of WRN-MSI SL interaction.

FIG. 6A and FIG. 6B depicts a series of graphs showing the results of a dual siRNA experiment in A549 cells with MSH2.

FIG. 7A and FIG. 7B are graphs depicting WRN and BLM knockdown levels.

FIG. 8A, FIG. 8B, and FIG. 8C depict MLH1 and MRE11 re-expressing cell lines do not rescue the WRN knockdown phenotype is MSI cells.

FIG. 9 is a series of fluorescent micrographs showing immunofluorescence of RKO and SW620 cells following WRN knockdown.

FIG. 10A, and FIG. 10B depict flow cytometry dot plots of cells transfected with WRN siRNA.

FIG. 11A, and FIG. 11B depict Rescue cell lines showing siRNA resistant WRN and relative caspase activity.

FIG. 12 depicts a series of graphs showing the effects of NSC compounds on MSI vs. MSS cell lines.

### DETAILED DESCRIPTION

We investigated the genomic mutational partners for which WRN may be synthetic lethal using a candidate approach. Expression of MMR proteins can be decreased, either through loss of function mutations or by promoter hypermethylation. MMR-deficient cells and tumors display high microsatellite instability (MSI).

We have determined that a SL interaction exists between WRN and MSI, particularly MSI-high. Interestingly, WRN is the only RECQ that has an exonuclease domain in addition to the helicase domain. Mutations in the helicase domain of WRN do not lead to the WS symptoms while mutations in the exonuclease domain do (Kamath-Loeb A S, Zavala-van Rankin D G, Flores-Morales J, Emond M J, Sidorova J M, Carnevale A, et al., Sci Rep 2017; 7:44081 doi 10.1038/srep44081). By contrast, using rescue experiments we show that the functional WRN helicase domain is important for maintaining viability in MSI cell lines. In this study, we found that cells with MSI depend on WRN for their survival, suggesting that inhibiting (e.g., by drugging) the WRN helicase domain or otherwise decreasing WRN helicase activity may be beneficial for treating cancer patients with tumors characterized by MSI, particularly MSI-high. As WS symptoms result from WRN exonuclease dysfunction, therapies that selectively target WRN helicase activity without affecting WRN exonuclease activity could be advantageous for inducing synthetic lethality in target cells, while minimizing potential deleterious off-target side effects. Nonetheless, therapeutic approaches that impact both domains—the helicase domain and the exonuclease domain (e.g., by reducing expression of WRN or degrading WRN or otherwise decreasing the expression or activity (including helicase activity)—are tractable.

### Definitions

Unless defined otherwise, all technical and scientific terms used herein have the same meaning as commonly understood by one of ordinary skill in the art to which the disclosure pertains. All patents, applications, published applications and other publications referenced herein are expressly incorporated by reference in their entireties unless stated otherwise. In the event that there are a plurality of definitions for a term herein, those in this section prevail unless stated otherwise.

As used herein, “a” or “an” may mean one or more than one.

“About” has its plain and ordinary meaning when read in light of the specification, and may be used, for example, when referring to a measurable value and may be meant to encompass variations of ±20% or ±10%, more preferably ±5%, even more preferably ±1%, and still more preferably ±0.1% from the specified value.

As used herein, a “subject” or “individual” refers to an animal that is the object of treatment, observation or experiment. “Animal” comprises cold- and warm-blooded vertebrates and invertebrates such as fish, shellfish, reptiles and, in particular, mammals. “Mammal” comprises, without limitation, mice, rats, rabbits, guinea pigs, dogs, cats, sheep, goats, cows, horses, primates, such as monkeys, chimpanzees, and apes, and, in particular, humans.

“Nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also comprises so-called “peptide nucleic acids,” which comprise naturally occurring or modified nucleic acid bases attached to a polyamide backbone. Nucleic acids can be either single stranded or double stranded. In some embodiments, a nucleic acid sequence encoding a fusion protein is provided. In some embodiments, the nucleic acid is RNA or DNA.

“Coding for” or “encoding” are used herein, and refers to the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

A nucleic acid “encoding” a polypeptide includes all nucleic acids that are degenerate versions of each other and that encode the same amino acid sequence.

“Vector” or “expression vector” includes nucleic acids used to introduce heterologous nucleic acids into a cell that has regulatory elements to provide expression of the heterologous nucleic acids in the cell. Vectors include but are not limited to plasmid, minicircles, yeast, and viral genomes. In some embodiments, the vectors are plasmid, minicircles, yeast, or viral genomes. In some embodiments, the vector is a viral vector. In some embodiments, the viral vector is a lentivirus. In some embodiments, the vector is an adeno-associated viral (AAV) vector. In some embodiments, the vector is for protein expression in a bacterial system such as E. coli. As used herein, the term “expression,” or “protein expression” refers to refers to the translation of a transcribed RNA molecule into a protein molecule. Protein expression may be characterized by its temporal, spatial, developmental, or morphological qualities as well as by quantitative or qualitative indications. In some embodiments, the protein or proteins are expressed such that the proteins are positioned for dimerization in the presence of a ligand.

As used herein, the term “regulatory element” refers to a DNA molecule having gene regulatory activity, e.g., one that has the ability to affect the transcription and/or translation of an operably linked transcribable DNA molecule. Regulatory elements such as promoters, leaders, introns, and transcription termination regions are DNA molecules that have gene regulatory activity and play an integral part in the overall expression of genes in living cells. Isolated regulatory elements, such as promoters, that function in plants are therefore useful for modifying plant phenotypes through the methods of genetic engineering.

“Percent (%) amino acid sequence identity” with respect to the amino acid sequences identified herein is defined as the percentage of amino acid residues in a candidate sequence that are identical with the amino acid residues in the reference sequence for each of the extracellular binding domain, hinge domain, transmembrane domain, and/or the signaling domain, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, comprising any algorithms needed to achieve maximal alignment over the full-length of the sequences being compared. For example, % amino acid sequence identity values generated using the WU-BLAST-2 computer program (Altschul; et al., Methods in Enzymology, 266:460-480 (1996)) uses several search parameters, most of which are set to the default values. Those that are not set to default values (e.g., the adjustable parameters) are set with the following values: overlap span=1, overlap fraction=0.125, word threshold (T)=11 and scoring matrix=BLOSUM62.

“Alkyl” refers to a straight or branched, saturated, aliphatic radical having the number of carbon atoms indicated (i.e., C₁₋₆ means one to six carbons). Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc.

“Alkylene” refers to a straight or branched, saturated, aliphatic radical having the number of carbon atoms indicated (i.e., C₁₋₆ means one to six carbons), and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ, where n is 1, 2, 3, 4, 5 or 6. Representative alkylene groups include, but are not limited to, methylene, ethylene, propylene, isopropylene, butylene, isobutylene, sec-butylene, pentylene and hexylene.

“Alkenyl” refers to a straight chain or branched hydrocarbon having at least 2 carbon atoms and at least one double bond and having the number of carbon atom indicated (i.e., C₂₋₆ means to two to six carbons). Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₅, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

“Alkynyl” refers to either a straight chain or branched hydrocarbon having at least 2 carbon atoms and at least one triple bond and having the number of carbon atom indicated (i.e., C₂₋₆ means to two to six carbons). Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl.

“Hydroxyalkyl” or “alkylhydroxy” refers to an alkyl group, as defined above, where at least one of the hydrogen atoms is replaced with a hydroxy group. As for the alkyl group, hydroxyalkyl or alkylhydroxy groups can have any suitable number of carbon atoms, such as C₁-C₆. Exemplary hydroxyalkyl groups include, but are not limited to, hydroxymethyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxyethyl, and the like.

“Halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁-C₆. For example, haloalkyl includes trifluoromethyl, fluoromethyl, 2,2,2-trifluoroethyl, etc. In some instances, the term “perfluoro” can be used to define a compound or radical where all the hydrogens are replaced with fluorine. For example, perfluoromethyl refers to 1,1,1-trifluoromethyl.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Aryl-alkyl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of aryl-alkyl groups include, but are not limited to, benzyl and phenylethyl.

### Methods of Decreasing Cell Proliferation

In one aspect, provided herein is a method for decreasing proliferation in a proliferative cell having a microsatellite instability (MSI), comprising decreasing the helicase activity of Werner syndrome ATP-dependent helicase (WRN) in the proliferative cell. In some embodiments, the proliferative cell is characterized as having high MSI (MSI-H), used interchangeably with MISH-high. Cells can be characterized as MSI or MSI-H according to any method known in the art (see, for example, Dudley, Jonathan C., et al., Clinical Cancer Research, 22(4): 813-820, 2016.). MSI-H is used to classify tumors as having a high frequency of MSI. A tumor can be classified as MSI or MSI-high using polymerase chain reaction (PCR) and/or immunohistochemistry (IHC) assays. As stated in Dudley et al. (supra), a tumor is classified as MSI-H by PCR if (i) there is a shift (usually downward) in the size of at least two microsatellite loci from a reference panel of five microsatellite loci in tumor relative to normal, where the reference panel can be the “Bethesda panel.” which includes two mononucleotide loci (BAT-25 and BAT-26) and three dinucleotide loci (D2S123, D5S346, and D17S250), or Promega Corporation's MSI Analysis System, which includes five mononucleotide loci (BAT-25, BAT-26, NR-21, NR-24, and MONO-27); or (ii) there is a shift in the size of 30% or more microsatellite loci from a reference panel of more than five microsatellite loci in tumor relative to normal. The MSI-H phenotype is associated with germline defects in the mismatch repair genes MLH1, MSH2, MSH6, and PMS2, and is the primary phenotype observed in tumors from patients with HNPCC/Lynch syndrome. A tumor is classified as MSI-H in IHC test if it show a loss of protein expression for at least 1 of the above 4 mismatch repair genes. Cells can be similarly classified as MSI-1 using the tests described herein for tumors

In some embodiments, a tumor or cell is classified as MSI-H using PCR to amplify the five microsatellite loci of the “Bethesda panel” (BAT-25, BAT-26, D2S123, D5S346, and D17S250) from both tumor tissue or cells and normal tissue or cells, wherein the tumor or cell is classified as MSI-H if there is a shift in the size of at least two of the microsatellite loci from the tumor tissue or cells relative to the normal tissue or cells. In some embodiments, the shift in size of the microsatellite loci is a downward shift.

In some embodiments, a tumor or cell is classified as MSI-H using PCR to amplify the five microsatellite loci of Promega Corporation's MSI Analysis System (BAT-25, BAT-26, NR-21, NR-24, and MONO-27) from both tumor tissue or cells and normal tissue or cells, wherein the tumor or cell is classified as MSI-H if there is a shift in the size of at least two of the microsatellite loci from the tumor tissue or cells relative to the normal tissue or cells. In some embodiments, the shift in size of the microsatellite loci is a downward shift.

In some embodiments, a tumor is classified as MSI-H using IHC to determine the expression level of the MMR proteins MLH1, MSH2, MSH6, and/or PMS2 in both tumor tissue and normal tissue, wherein the tumor is classified as MSI-H if there is a loss of protein expression for at least one of the MMR proteins in the tumor tissue relative to the normal tissue. In some embodiments, the loss of protein expression is a decrease of at least 20% (such as a decrease of 30%, 40%, 50%, 60%, 70%, 80%, 90%, 95%, 99%, or more).

In contrast, a tumor is classified as MSI-L by PCR if (i) there is a shift in the size of one microsatellite locus from a reference panel of five microsatellite loci in tumor relative to normal, where the reference panel can be the “Bethesda panel” or Promega Corporation's MSI Analysis System; or (ii) there is a shift in the size of less than 30% microsatellite loci from a reference panel of more than five microsatellite loci in tumor relative to normal. MSI-L tumors are thought to represent a distinct mutator phenotype with potentially different molecular etiology than MSI-H tumors (Thibodeau, 1998; Wu et al., 1999, Am J Hum Genetics 65:1291-1298). Cells can be similarly classified as MSI-L using the tests described herein for tumors.

In some embodiments, the proliferative cell is a mammalian cell. In some embodiments, the mammalian cell is a primate cell, such as a human cell. In some embodiments, the proliferative cell is a veterinary animal cell. In some embodiments, the proliferative cell is a cancer cell. In some embodiments, the proliferative cell is a circulating tumor cell.

In some embodiments, provided herein is a method for decreasing proliferation in a proliferative cell having an MSI (or MSI-H), comprising delivering into the proliferative cell an inhibitor of WRN such that the helicase activity of WRN is decreased in the proliferative cell. In some embodiments, the WRN inhibitor does not decrease the exonuclease activity of WRN in the proliferative cell. In some embodiments, the WRN inhibitor is a small molecule inhibitor. In some embodiments, the WRN inhibitor is an antibody drug conjugate (ADC) comprising an antibody conjugated to a WRN inhibitor. In some embodiments, the antibody in the ADC targets the ADC to the proliferative cell.

In some embodiments, provided herein is a method for decreasing proliferation in a proliferative cell having an MSI (or MSI-H), comprising delivering into the proliferative cell a small molecule inhibitor of WRN such that the helicase activity of WRN is decreased in the proliferative cell. In some embodiments, the small molecule WRN inhibitor does not decrease the exonuclease activity of WRN in the proliferative cell. In some embodiments, the small molecule inhibitor has the formula:

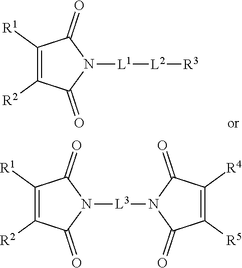

or a pharmaceutically acceptable salt thereof,

wherein:
L¹ is C₁₋₄ alkylene;
L² is O, S, OC(O), OSO₂, OC(O)O, or OC(O)NH;
L³ is C₁₋₈ alkylene;
R¹, R², R⁴, and R⁵ are each independently H, halogen, C₁₋₄ alkyl, or C₁₋₄ haloalkyl; and
R³ is H, C₁₋₆ alkyl, C₂₋₆ alkenyl, C₂₋₆ alkynyl, C₁₋₆ hydroxyalkyl, C₁₋₆ haloalkyl, C₆₋₁₂ aryl,
   or C₆₋₁₂ aryl-C₁₋₄ alkyl, each of which is optionally substituted with halogen, C₁₋₄ alkyl, or C₁₋₄ haloalkyl.

In some embodiments, L¹ is C₁₋₄ alkylene. The C₁₋₄ alkylene of L¹ can be methylene (CH₂), ethylene, propylene, isopropylene, butylene, isobutylene, or sec-butylene. In some embodiments, L¹ is CH₂.

In some embodiments, L² is OC(O).

In some embodiments, L³ is C₁₋₈ alkylene. In some embodiments, L³ is C₁₋₆ alkylene. The C₁₋₈ alkylene of L³ can be methylene, ethylene, propylene, isopropylene, butylene, isobutylene, sec-butylene, pentenylene, 2,2-dimethylpropylene (CH₂C(CH₃)₂CH₂), pentylene, hexylene, heptylene, or octylene. In some embodiments, L³ is CH₂C(CH₃)₂CH₂.

In some embodiments, R¹, R², R⁴, and R⁵ are each independently H or halogen. In some embodiments, R¹ and R² are each independently H or halogen. In some embodiments, R¹, R², R⁴, and R⁵ are each H. In some embodiments, R¹ and R² are each H. In some embodiments, R¹, R², R⁴, and R⁵ are each halogen. In some embodiments, R¹ and R² are each halogen. Halogen can be F, Cl, Br, or I. In some embodiments, R¹, R², R⁴, and R⁵ are each Cl. In some embodiments, R¹ and R² are each Cl.

In some embodiments, R³ is H, C₁₋₆ alkyl, C₂₋₆ alkenyl, C₂₋₆ alkynyl, C₁₋₆ hydroxyalkyl, C₁₋₆ haloalkyl, phenyl, or benzyl, each of which is optionally substituted with halogen, C₁₋₄ alkyl, or C₁₋₄ haloalkyl. In some embodiments, R³ is C₁₋₆ alkyl. The C₁₋₆ alkyl of R³ can be methyl, ethyl, propyl, isopropyl, butyl, isobutyl, sec-butyl, tert-butyl, pentyl, isopentyl, or hexyl. In some embodiments, R³ is ethyl.

In some embodiments, the small molecule inhibitor is selected from the group consisting of NSC 617145 and NSC 19630. NSC 617145 has the formula:

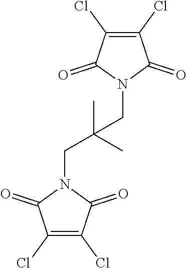 [SMILES: CC(C)(CN1C(=O)C(Cl)=C(Cl)C1=O)CN1C(=O)C(Cl)=C(Cl)C1=O]

and NSC 19630 has the formula:

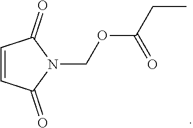

In some embodiments, the small molecule inhibitor is other than NSC 617145, NSC 19630, and ML-216. ML-216 has the formula:

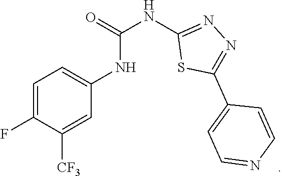 [SMILES: O=C(Nc1ccc(F)c(C(F)(F)F)c1)Nc1nnc(-c2ccncc2)s1]

In some embodiments, provided herein is a method for decreasing proliferation in a proliferative cell having an MSI (or MSI-H), comprising contacting the proliferative cell with an ADC comprising an antibody conjugated to a WRN inhibitor, such that the helicase activity of WRN is decreased in the proliferative cell. In some embodiments, the ADC does not decrease the exonuclease activity of WRN in the proliferative cell. In some embodiments, the antibody in the ADC targets the ADC to the proliferative cell.

In another aspect, provided herein is a method for decreasing proliferation in a proliferative cell having an MSI (or MSI-H), comprising delivering into the proliferative cell a nuclease capable of modifying the genome of the proliferative cell such that the helicase activity of WRN in the proliferative cell is decreased, or a nucleic acid encoding the nuclease. In some embodiments, the modification to the genome does not decrease the exonuclease activity of WRN in the proliferative cell. In some embodiments, the nuclease is a transcription activator-like effector nuclease (TALEN) or zinc-finger nucleases (ZFN) targeting a genomic sequence within or near an endogenous WRN gene locus. In some embodiments, the nuclease is an RNA-guided endonuclease (RGEN), and the method further comprises delivering into the proliferative cell a gRNA comprising a spacer sequence that is complementary to a genomic sequence within or near an endogenous WRN gene locus, or a nucleic acid encoding the gRNA. In some embodiments, the genome of the proliferative cell is modified by non-homologous end joining (NHEJ).

In some embodiments, provided herein is a method for decreasing proliferation in a proliferative cell having an MSI (or MSI-H), comprising delivering into the proliferative cell a TALEN or ZFN targeting a genomic sequence within or near an endogenous WRN gene locus, such that such that the helicase activity of WRN in the proliferative cell is decreased, or a nucleic acid encoding the TALEN or ZFN. In some embodiments, the exonuclease activity of WRN in the proliferative cell is not decreased.

In some embodiments, provided herein is a method for decreasing proliferation in a proliferative cell having an MSI (or MSI-H), comprising delivering into the proliferative cell a) a gRNA comprising a spacer sequence that is complementary to a genomic sequence within or near an endogenous WRN gene locus, or a nucleic acid encoding the gRNA; and b) an RNA-guided endonuclease (RGEN), or a nucleic acid encoding the RGEN, such that such that the helicase activity of WRN in the proliferative cell is decreased. In some embodiments, the exonuclease activity of WRN in the proliferative cell is not decreased. In some embodiments, the nucleic acid encoding the gRNA is contained in an Adeno Associated Virus (AAV) vector and/or the nucleic acid encoding the RGEN is contained in an AAV vector. In some embodiments, the spacer sequence is complementary to a genomic sequence within a coding region of the endogenous WRN gene. In some embodiments, the genome of the proliferative cell is modified by non-homologous end joining (NHEJ).

In some embodiments, according to any of the methods described herein comprising delivering into a proliferative cell a gRNA or nucleic acid encoding the gRNA and an RGEN or nucleic acid encoding the RGEN, the method further comprises delivering into the proliferative cell a donor template comprising a donor nucleic acid, wherein the donor template is configured such that the donor nucleic acid is capable of being inserted into the WRN gene locus by homologous recombination. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in the WRN helicase domain that decreases WRN helicase activity. In some embodiments, the donor nucleic acid encodes a deletion in the WRN helicase domain that decreases WRN helicase activity. In some embodiments, the donor template is contained in an AAV vector.

In some embodiments, according to any of the methods described herein comprising delivering into a proliferative cell a gRNA or nucleic acid encoding the gRNA and an RGEN or nucleic acid encoding the RGEN, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof. In some embodiments, the RGEN is Cas9.

In some embodiments, according to any of the methods described herein comprising delivering into a proliferative cell a gRNA or nucleic acid encoding the gRNA and an RGEN or nucleic acid encoding the RGEN, the method comprises delivering into the proliferative cell a nucleic acid encoding the RGEN. In some embodiments, the nucleic acid encoding the RGEN is a ribonucleic acid (RNA), such as an mRNA. In some embodiments, the method comprises delivering into the proliferative cell the gRNA. In some embodiments, the RNA encoding the RGEN is linked to the gRNA via a covalent bond. In some embodiments, the nucleic acid encoding the RGEN is a deoxyribonucleic acid (DNA), such as a DNA plasmid. In some embodiments, the nucleic acid encoding the RGEN is formulated in a liposome or lipid nanoparticle. In some embodiments, the liposome or lipid nanoparticle encapsulates the gRNA.

In some embodiments, according to any of the methods described herein comprising delivering into a proliferative cell a gRNA or nucleic acid encoding the gRNA and an RGEN or nucleic acid encoding the RGEN, the method comprises delivering into the proliferative cell the RGEN. In some embodiments, the method comprises delivering into the proliferative cell the gRNA. In some embodiments, the RGEN is pre-complexed with the gRNA, forming a Ribonucleoprotein (RNP) complex.

In another aspect, provided herein is a method for decreasing proliferation in a proliferative cell having an MSI, comprising delivering into the proliferative cell a nucleic acid construct comprising a donor nucleic acid, wherein the nucleic acid construct is configured such that insertion of the donor nucleic acid into the genome of the proliferative cell by homologous recombination decreases the helicase activity of WRN in the proliferative cell. In some embodiments, the modification does not decrease WRN exonuclease activity in the proliferative cell. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in the WRN helicase domain that decreases WRN helicase activity. In some embodiments, the donor nucleic acid encodes a deletion in the WRN helicase domain that reduces or eliminates WRN helicase activity. In some embodiments, the nucleic acid construct is an AAV vector. In some embodiments, the AAV vector comprises two homology arms having sequences identical or substantially homologous (such at least about any of 90%, 95%, 96%, 97%, 98%, or 99% homologous) to regions of the endogenous WRN gene. In some embodiments, the AAV vector is an AAV clade F vector.

In some embodiments, according to any of the methods described herein for decreasing proliferation in a proliferative cell having an MSI (or MSI-H), the proliferative cell comprises one or more (such as any of 2, 3, 4, or 5) MSI markers selected from the group consisting of BAT25, BAT26, D2S123, D5S346, and D175250. In some embodiments, the proliferative cell comprises one MSI marker selected from the group consisting of BAT25, BAT26, D2S123, D5S346, and D175250. In some embodiments, the proliferative cell comprises two MSI markers selected from the group consisting of BAT25, BAT26, D2S123, D5S346, and D17S250. In some embodiments, the proliferative cell comprises three MSI markers selected from the group consisting of BAT25, BAT26, D2S123, D5S346, and D17S250. In some embodiments, the proliferative cell comprises four MSI markers selected from the group consisting of BAT25, BAT26, D2S123, D5S346, and D17S250. In some embodiments, the proliferative cell comprises five MSI markers selected from the group consisting of BAT25, BAT26, D2S123, D5S346, and D17S250.

In some embodiments, according to any of the methods described herein for decreasing proliferation in a proliferative cell having an MSI (or MSI-H), the proliferative cell comprises one or more mutations that impair DNA mismatch repair. In some embodiments, the one or more mutations comprise a mutation in a MutS homolog and/or a mutation in a MutL homolog. In some embodiments, the MutS homolog is selected from the group consisting of MSH2, MSH3, and MSH6. In some embodiments, the MutL homolog is selected from the group consisting of MLH1, MLH3, PMS1, and PMS2. In some embodiments, the proliferative cell comprises a mutation in at least one MMR protein selected from MLH1, MSH2, MSH6, and PMS2. In some embodiments, the proliferative cell comprises a mutation in MLH1. In some embodiments, the proliferative cell comprises a mutation in MSH2. In some embodiments, the proliferative cell comprises a mutation in MSH6. In some embodiments, the proliferative cell comprises a mutation in PMS2. In some embodiments, the proliferative cell comprises a mutation in MLH1 and a mutation in MSH2. In some embodiments, the proliferative cell comprises a mutation in MLH1 and a mutation in MSH6. In some embodiments, the proliferative cell comprises a mutation in MLH1 and a mutation in PMS2. In some embodiments, the proliferative cell comprises a mutation in MSH2 and a mutation in MSH6. In some embodiments, the proliferative cell comprises a mutation in MSH2 and a mutation in PMS2. In some embodiments, the proliferative cell comprises a mutation in MSH6 and a mutation in PMS2. In some embodiments, the proliferative cell comprises a mutation in MLH1, a mutation in MSH2, and a mutation in MSH6. In some embodiments, the proliferative cell comprises a mutation in MLH1, a mutation in MSH2, and a mutation in PMS2. In some embodiments, the proliferative cell comprises a mutation in MLH1, a mutation in MSH6, and a mutation in PMS2. In some embodiments, the proliferative cell comprises a mutation in MSH2, a mutation in MSH6, and a mutation in PMS2. In some embodiments, the proliferative cell comprises a mutation in MLH1, a mutation in MSH2, a mutation in MSH6, and a mutation in PMS2.

In some embodiments, according to any of the methods described herein for decreasing proliferation in a proliferative cell having an MSI (or MSI-H), the proliferative cell comprises one or more markers of DNA damage. In some embodiments, the one or more markers of DNA damage are selected from the group consisting of high p21 expression and high γH2AX expression. In some embodiments, the proliferative cell comprises high p21 expression. In some embodiments, the proliferative cell comprises high γH2AX expression. In some embodiments, the proliferative cell comprises high p21 expression and high γH2AX expression.

In some embodiments, according to any of the methods described herein for decreasing proliferation in a proliferative cell having an MSI (or MSI-H), decreasing proliferation in the proliferative cell comprises inducing cell cycle arrest in the proliferative cell. In some embodiments, the proliferative cell is induced to be arrested in G1. In some embodiments, the proliferative cell is induced to be arrested in G2.

In some embodiments, according to any of the methods described herein for decreasing proliferation in a proliferative cell having an MSI (or MSI-H), decreasing proliferation in the proliferative cell comprises inducing apoptosis in the proliferative cell. In some embodiments, the proliferative cell is induced to have increased caspase activity.

In some embodiments, according to any of the methods described herein for decreasing proliferation in a proliferative cell having an MSI (or MSI-H), the method is carried out in vivo. In some embodiments, the proliferative cell is present in a mammal. In some embodiments, the mammal is a primate, such as a human. In some embodiments, the proliferative cell is present in a veterinary animal. In some embodiments, according to any of the methods described herein for decreasing proliferation in a proliferative cell having an MSI, the method is carried out ex vivo. In some embodiments, according to any of the methods described herein for decreasing proliferation in a proliferative cell having an MSI (or MSI-H), the method is carried out in vitro. In some embodiments, the proliferative cell is derived from a mammal. In some embodiments, the mammal is a primate, such as a human. In some embodiments, the proliferative cell is derived from a veterinary animal.

In some embodiments, according to any of the methods described herein for decreasing proliferation in a proliferative cell having an MSI (or MSI-H), the proliferative cell is a cancer cell. In some embodiments, the cancer includes, without limitation, colon cancer, gastric cancer, endometrium cancer, ovarian cancer, hepatobiliary tract cancer, urinary tract cancer, brain cancer, and skin cancer.

In another aspect, provided herein is a method for decreasing proliferation in a proliferative cell having an MSI (or MSI-H), comprising decreasing the expression and/or activity of WRN in the proliferative cell. In some embodiments, the method comprises delivering into the proliferative cell an inhibitor of WRN. In some embodiments, the WRN inhibitor is a small molecule inhibitor. In some embodiments, the WRN inhibitor is an antibody drug conjugate (ADC) comprising an antibody conjugated to a WRN inhibitor. In some embodiments, the antibody in the ADC targets the ADC to the proliferative cell. In some embodiments, the method comprises delivering into the proliferative cell a nuclease, or a nucleic acid encoding the nuclease, capable of modifying the genome of the proliferative cell such that the expression and/or activity of WRN in the proliferative cell is decreased. In some embodiments, the nuclease is a TALEN or ZFN targeting a genomic sequence within or near an endogenous WRN gene locus. In some embodiments, the nuclease is an RGEN, and the method further comprises delivering into the proliferative cell a gRNA comprising a spacer sequence that is complementary to a genomic sequence within or near an endogenous WRN gene locus, or a nucleic acid encoding the gRNA. In some embodiments, the method further comprises delivering into the proliferative cell a donor template comprising a donor nucleic acid, wherein the donor template is configured such that the donor nucleic acid is capable of being inserted into the WRN gene locus by homologous recombination. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in WRN that decreases its activity. In some embodiments, the donor nucleic acid encodes a deletion in WRN that decreases its activity.

In another aspect, provided herein is a method for decreasing proliferation in a proliferative cell having an MSI (or MSI-H), comprising delivering into the proliferative cell an inhibitory nucleic acid targeting WRN mRNA, or a nucleic acid encoding the inhibitory nucleic acid, such that the expression of WRN is decreased in the proliferative cell. In some embodiments, the inhibitory nucleic acid comprises a short interfering RNA (siRNA), a microRNA (miRNA), or an antisense oligonucleotide.

In another aspect, provided herein is a method for decreasing proliferation in a proliferative cell having an MSI (or MSI-H), comprising delivering into the proliferative cell a proteolysis targeting chimera (PROTAC) that targets WRN for ubiquitination and proteolytic degradation. In some embodiments, the PROTAC comprises an E3 ubiquitin ligase ligand coupled via a linker to a WRN ligand.

### Engineered Cells

In one aspect, provided herein is an engineered cell, such as an engineered mammalian cell (e.g., a proliferative cell, such as a cancer cell), having an MSI (or MSI-H), wherein the engineered cell has been modified to decrease the helicase activity of WRN as compared to a corresponding unmodified cell. In some embodiments, the engineered cell does not have decreased WRN exonuclease activity as compared to a corresponding unmodified cell. In some embodiments, the engineered cell is a mammalian cell. In some embodiments, the mammalian cell is a primate cell, such as a human cell. In some embodiments, the engineered cell is a veterinary animal cell. In some embodiments, the engineered cell is a cancer cell. In some embodiments, the engineered cell is a circulating tumor cell.

In some embodiments, provided herein is an engineered cell prepared by modifying an input cell having an MSI (or MSI-H), wherein the modification comprises delivering into the input cell a nuclease capable of modifying the genome of the input cell such that the helicase activity of WRN in the input cell is decreased, or a nucleic acid encoding the nuclease. In some embodiments, the genome modification does not decrease WRN exonuclease activity in the input cell. In some embodiments, the nuclease is a transcription activator-like effector nuclease (TALEN) or zinc-finger nucleases (ZFN) targeting a genomic sequence within or near an endogenous WRN gene locus. In some embodiments, the nuclease is an RNA-guided endonuclease (RGEN), and the modification further comprises delivering into the input cell a gRNA comprising a spacer sequence that is complementary to a genomic sequence within or near an endogenous WRN gene locus, or a nucleic acid encoding the gRNA. In some embodiments, the genome of the input cell is modified by non-homologous end joining (NHEJ).

In some embodiments, provided herein is an engineered cell prepared by modifying an input cell having an MSI (or MSI-H), wherein the modification comprises delivering into the input cell a TALEN or ZFN, or nucleic acid encoding the TALEN or ZFN, targeting a genomic sequence within or near an endogenous WRN gene locus, such that the helicase activity of WRN in the input cell is decreased. In some embodiments, the modification does not decrease WRN exonuclease activity in the input cell.

In some embodiments, provided herein is an engineered cell prepared by modifying an input cell having an MSI, wherein the modification comprises delivering into the input cell a) a gRNA comprising a spacer sequence that is complementary to a genomic sequence within or near an endogenous WRN gene locus, or a nucleic acid encoding the gRNA; and b) an RNA-guided endonuclease (RGEN), or a nucleic acid encoding the RGEN, such that the helicase activity of WRN in the input cell is decreased. In some embodiments, the modification does not decrease WRN exonuclease activity in the input cell. In some embodiments, the nucleic acid encoding the gRNA is contained in an Adeno Associated Virus (AAV) vector and/or the nucleic acid encoding the RGEN is contained in an AAV vector. In some embodiments, the spacer sequence is complementary to a genomic sequence within a coding region of the endogenous WRN gene. In some embodiments, the genome of the input cell is modified by non-homologous end joining (NHEJ).

In some embodiments, according to any of the engineered cells described herein prepared by a method comprising delivering into an input cell a gRNA or nucleic acid encoding the gRNA and an RGEN or nucleic acid encoding the RGEN, the method further comprises delivering into the input cell a donor template comprising a donor nucleic acid, wherein the donor template is configured such that the donor nucleic acid is capable of being inserted into the WRN gene locus by homologous recombination. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in the WRN helicase domain that decreases WRN helicase activity. In some embodiments, the donor nucleic acid encodes a deletion in the WRN helicase domain that reduces or eliminates WRN helicase activity. In some embodiments, the donor template is contained in an AAV vector.

In some embodiments, according to any of the engineered cells described herein prepared by a method comprising delivering into an input cell a gRNA or nucleic acid encoding the gRNA and an RGEN or nucleic acid encoding the RGEN, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof. In some embodiments, the RGEN is Cas9.

In some embodiments, according to any of the engineered cells described herein prepared by a method comprising delivering into an input cell a gRNA or nucleic acid encoding the gRNA and an RGEN or nucleic acid encoding the RGEN, the method comprises delivering into the input cell a nucleic acid encoding the RGEN. In some embodiments, the nucleic acid encoding the RGEN is a ribonucleic acid (RNA), such as an mRNA. In some embodiments, the method comprises delivering into the input cell the gRNA. In some embodiments, the RNA encoding the RGEN is linked to the gRNA via a covalent bond. In some embodiments, the nucleic acid encoding the RGEN is a deoxyribonucleic acid (DNA), such as a DNA plasmid. In some embodiments, the nucleic acid encoding the RGEN is formulated in a liposome or lipid nanoparticle. In some embodiments, the liposome or lipid nanoparticle encapsulates the gRNA.

In some embodiments, according to any of the engineered cells described herein prepared by a method comprising delivering into an input cell a gRNA or nucleic acid encoding the gRNA and an RGEN or nucleic acid encoding the RGEN, the method comprises delivering into the input cell the RGEN. In some embodiments, the method comprises delivering into the input cell the gRNA. In some embodiments, the RGEN is pre-complexed with the gRNA, forming a Ribonucleoprotein (RNP) complex.

In another aspect, provided herein is an engineered cell prepared by modifying an input cell having an MSI, wherein the modification comprises delivering into the input cell a nucleic acid construct comprising a donor nucleic acid, and wherein the nucleic acid construct is configured such that insertion of the donor nucleic acid into the genome of the input cell by homologous recombination decreases the helicase activity of WRN in the input cell. In some embodiments, the modification does not decrease WRN exonuclease activity in the input cell. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in the WRN helicase domain that decreases WRN helicase activity. In some embodiments, the donor nucleic acid encodes a deletion in the WRN helicase domain that reduces or eliminates WRN helicase activity. In some embodiments, the nucleic acid construct is an AAV vector. In some embodiments, the AAV vector comprises two homology arms having sequences identical or substantially homologous (such at least about any of 90%, 95%, 96%, 97%, 98%, or 99% homologous) to regions of the endogenous WRN gene. In some embodiments, the AAV vector is an AAV clade F vector.

In some embodiments, according to any of the engineered cells described herein, the engineered cell comprises one or more (such as any of 2, 3, 4, or 5) MSI markers selected from the group consisting of BAT25, BAT26, D2S123, D5S346, and D17S250. In some embodiments, the engineered cell comprises one MSI marker selected from the group consisting of BAT25, BAT26, D2S123, D5S346, and D17S250. In some embodiments, the engineered cell comprises two MSI markers selected from the group consisting of BAT25, BAT26, D2S123, D5S346, and D17S250. In some embodiments, the engineered cell comprises three MSI markers selected from the group consisting of BAT25, BAT26, D2S123, D5S346, and D17S250. In some embodiments, the engineered cell comprises four MSI markers selected from the group consisting of BAT25, BAT26, D2S123, D5S346, and D17S250. In some embodiments, the engineered cell comprises five MSI markers selected from the group consisting of BAT25, BAT26, D2S123, D5S346, and D17S250.

In some embodiments, according to any of the engineered cells described herein, the engineered cell comprises one or more mutations that impair DNA mismatch repair. In some embodiments, the one or more mutations comprise a mutation in a MutS homolog and/or a mutation in a MutL homolog. In some embodiments, the MutS homolog is selected from the group consisting of MSH2, MSH3, and MSH6. In some embodiments, the MutL homolog is selected from the group consisting of MLH1, MLH3, PMS1, and PMS2. In some embodiments, the engineered cell comprises a mutation in MLH1. In some embodiments, the engineered cell comprises a mutation in MSH2. In some embodiments, the engineered cell comprises a mutation in PMS2. In some embodiments, the engineered cell comprises a mutation in MLH1 and a mutation in MSH2. In some embodiments, the engineered cell comprises a mutation in MLH1 and a mutation in PMS2. In some embodiments, the proliferative cell comprises a mutation in at least one MMR protein selected from MLH1, MSH2, MSH6, and PMS2.

In some embodiments, according to any of the engineered cells described herein, the engineered cell comprises one or more markers of DNA damage. In some embodiments, the one or more markers of DNA damage are selected from the group consisting of high p21 expression and high γH2AX expression. In some embodiments, the engineered cell comprises high p21 expression. In some embodiments, the engineered cell comprises high γH2AX expression. In some embodiments, the engineered cell comprises high p21 expression and high γH2AX expression.

In some embodiments, according to any of the engineered cells described herein, the engineered cell is in a state of cell cycle arrest. In some embodiments, the engineered cell is arrested in G1. In some embodiments, the engineered cell is arrested in G2.

In some embodiments, according to any of the engineered cells described herein, the engineered cell is in a state of apoptosis. In some embodiments, the engineered cell has increased caspase activity.

In some embodiments, according to any of the engineered cells described herein, the modification to prepare the engineered cell is carried out in vivo. In some embodiments, the engineered cell is present in a mammal. In some embodiments, the mammal is a primate, such as a human. In some embodiments, according to any of the engineered cells described herein, the modification to prepare the engineered cell is carried out ex vivo. In some embodiments, according to any of the engineered cells described herein, the modification to prepare the engineered cell is carried out in vitro. In some embodiments, the engineered cell is derived from a mammal. In some embodiments, the mammal is a primate, such as a human.

In some embodiments, according to any of the engineered cells described herein, the engineered cell is a cancer cell. In some embodiments, the cancer includes, without limitation, colon cancer, gastric cancer, endometrium cancer, ovarian cancer, hepatobiliary tract cancer, urinary tract cancer, brain cancer, and skin cancer.

In another aspect, provided herein is an engineered cell, such as an engineered mammalian cell (e.g., a proliferative cell, such as a cancer cell), having an MSI (or MSI-H), wherein the engineered cell has been modified to decrease the expression and/or activity of WRN as compared to a corresponding unmodified cell. In some embodiments, the engineered cell is prepared by modifying an input cell having an MSI, wherein the modification comprises delivering into the input cell a nuclease capable of modifying the genome of the input cell such that (i) the expression of WRN in the input cell is decreased or (ii) the activity of WRN in the input cell is decreased, or a nucleic acid encoding the nuclease. In some embodiments, the engineered cell is prepared by modifying an input cell having an MSI, wherein the modification comprises delivering into the input cell a nucleic acid construct comprising a donor nucleic acid, and wherein the nucleic acid construct is configured such that insertion of the donor nucleic acid into the genome of the input cell by homologous recombination (i) decreases the expression of WRN in the input cell, or (ii) decreases the activity of WRN in the input cell.

### Methods of Genome Editing

In one aspect, provided herein is a method of editing the genome of a cell (e.g., a proliferative cell, such as a cancer cell) having an MSI (or MSI-H), in particular, editing the cell genome to decrease the helicase activity of WRN. In some embodiments, the WRN exonuclease activity in the cell is not decreased. In some embodiments, the cell is a mammalian cell. In some embodiments, the cell is a primate cell, such as a human cell. In some embodiments, the cell is a veterinary animal cell. In some embodiments, the cell is a cancer cell. In some embodiments, the cell is a circulating tumor cell.

In some embodiments, provided herein is a method of editing the genome of a cell having an MSI (or MSI-H) to produce an engineered cell having decreased WRN helicase activity, the method comprising delivering into the cell a nuclease capable of modifying the genome of the cell such that the helicase activity of WRN in the cell is decreased, or a nucleic acid encoding the nuclease. In some embodiments, the WRN exonuclease activity in the cell is not decreased. In some embodiments, the nuclease is a transcription activator-like effector nuclease (TALEN) or zinc-finger nucleases (ZFN) targeting a genomic sequence within or near an endogenous WRN gene locus. In some embodiments, the nuclease is an RNA-guided endonuclease (RGEN), and the method further comprises delivering into the cell a gRNA comprising a spacer sequence that is complementary to a genomic sequence within or near an endogenous WRN gene locus, or a nucleic acid encoding the gRNA. In some embodiments, the genome of the cell is modified by non-homologous end joining (NHEJ).

In some embodiments, provided herein is a method of editing the genome of a cell having an MSI (or MSI-H) to produce an engineered cell having decreased WRN helicase activity, the method comprising delivering into the cell a TALEN or ZFN targeting a genomic sequence within or near an endogenous WRN gene locus, such that the helicase activity of WRN in the cell is decreased, or a nucleic acid encoding the nuclease. In some embodiments, the WRN exonuclease activity in the cell is not decreased.

In some embodiments, provided herein is a method of editing the genome of a cell having an MSI (or MSI-H) to produce an engineered cell having decreased WRN helicase activity, the method comprising delivering into the cell a) a gRNA comprising a spacer sequence that is complementary to a genomic sequence within or near an endogenous WRN gene locus, or a nucleic acid encoding the gRNA; and b) an RNA-guided endonuclease (RGEN), or a nucleic acid encoding the RGEN such that the helicase activity of WRN in the cell is decreased. In some embodiments, the WRN exonuclease activity in the cell is not decreased. In some embodiments, the nucleic acid encoding the gRNA is contained in an Adeno Associated Virus (AAV) vector and/or the nucleic acid encoding the RGEN is contained in an AAV vector. In some embodiments, the spacer sequence is complementary to a genomic sequence within a coding region of the endogenous WRN gene. In some embodiments, the genome of the cell is modified by non-homologous end joining (NHEJ).

In some embodiments, according to any of the methods described herein for editing the genome of a cell comprising delivering into the cell a gRNA or nucleic acid encoding the gRNA and an RGEN or nucleic acid encoding the RGEN, the method further comprises delivering into the cell a donor template comprising a donor nucleic acid, wherein the donor template is configured such that the donor nucleic acid is capable of being inserted into the WRN gene locus by homologous recombination. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in the WRN helicase domain that decreases WRN helicase activity. In some embodiments, the donor nucleic acid encodes a deletion in the WRN helicase domain that reduces or eliminates WRN helicase activity. In some embodiments, the donor template is contained in an AAV vector.

In some embodiments, according to any of the methods described herein for editing the genome of a cell comprising delivering into the cell a gRNA or nucleic acid encoding the gRNA and an RGEN or nucleic acid encoding the RGEN, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof. In some embodiments, the RGEN is Cas9.

In some embodiments, according to any of the methods described herein for editing the genome of a cell comprising delivering into the cell a gRNA or nucleic acid encoding the gRNA and an RGEN or nucleic acid encoding the RGEN, the method comprises delivering into the cell a nucleic acid encoding the RGEN. In some embodiments, the nucleic acid encoding the RGEN is a ribonucleic acid (RNA), such as an mRNA. In some embodiments, the method comprises delivering into the cell the gRNA. In some embodiments, the RNA encoding the RGEN is linked to the gRNA via a covalent bond. In some embodiments, the nucleic acid encoding the RGEN is a deoxyribonucleic acid (DNA), such as a DNA plasmid. In some embodiments, the nucleic acid encoding the RGEN is formulated in a liposome or lipid nanoparticle. In some embodiments, the liposome or lipid nanoparticle encapsulates the gRNA.

In some embodiments, according to any of the methods described herein for editing the genome of a cell comprising delivering into the cell a gRNA or nucleic acid encoding the gRNA and an RGEN or nucleic acid encoding the RGEN, the method comprises delivering into the cell the RGEN. In some embodiments, the method comprises delivering into the cell the gRNA. In some embodiments, the RGEN is pre-complexed with the gRNA, forming a Ribonucleoprotein (RNP) complex.

In another aspect, provided herein is a method of editing the genome of a cell having an MSI (or MSI-H) to produce an engineered cell having decreased WRN helicase activity, the method comprising delivering into the cell a nucleic acid construct comprising a donor nucleic acid, wherein the nucleic acid construct is configured such that insertion of the donor nucleic acid into the genome of the cell by homologous recombination decreases the helicase activity of WRN in the cell. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in the WRN helicase domain that decreases WRN helicase activity. In some embodiments, the donor nucleic acid encodes a deletion in the WRN helicase domain that reduces or eliminates WRN helicase activity. In some embodiments, the nucleic acid construct is an AAV vector. In some embodiments, the AAV vector comprises two homology arms having sequences identical or substantially homologous (such at least about any of 90%, 95%, 96%, 97%, 98%, or 99% homologous) to regions of the endogenous WRN gene. In some embodiments, the AAV vector is an AAV clade F vector.

In another aspect, provided herein is a method of editing the genome of a cell (e.g., a proliferative cell, such as a cancer cell) having an MSI (or MSI-H), in particular, editing the cell genome to decrease the expression and/or activity of WRN. In some embodiments, the method comprises delivering into the cell a nuclease capable of modifying the genome of the cell such that (i) the expression of WRN in the cell is decreased or (ii) the activity of WRN in the cell is decreased, or a nucleic acid encoding the nuclease. In some embodiments, the method comprises delivering into the cell a nucleic acid construct comprising a donor nucleic acid, wherein the nucleic acid construct is configured such that insertion of the donor nucleic acid into the genome of the cell by homologous recombination (i) decreases the expression of WRN in the cell, or (ii) decreases the activity of WRN in the cell.

### Methods of Treatment

In some embodiments, provided herein is a method for treating a disease or condition in an individual in need thereof, wherein the disease or condition is characterized by a cell having an MSI (or MSI-H), the method comprising administering to the individual a pharmaceutical agent effective for decreasing the helicase activity of WRN in the cell. In some embodiments, administration of the pharmaceutical agent to the individual does not decrease WRN exonuclease activity in the cell. In some embodiments, the disease or condition is a proliferative disease, and the cell is a proliferative cell having an MSI. In some embodiments, the cell is a cancer cell. In some embodiments, the cell is a circulating tumor cell.

In another aspect, provided herein is a method for treating a proliferative disease in an individual in need thereof, the proliferative disease being characterized by proliferative cells having an MSI (or MSI-H), comprising administering to the individual a pharmaceutical composition comprising an inhibitor of WRN such that the helicase activity of WRN is decreased in the proliferative cells. In some embodiments, the WRN inhibitor does not decrease the exonuclease activity of WRN in the proliferative cells. In some embodiments, the WRN inhibitor is a small molecule inhibitor. In some embodiments, the WRN inhibitor is an ADC comprising an antibody conjugated to a WRN inhibitor. In some embodiments, the antibody in the ADC targets the ADC to the proliferative cells.

In some embodiments, provided herein is a method for treating a proliferative disease in an individual in need thereof, the proliferative disease being characterized by proliferative cells having an MSI (or MSI-H), comprising administering to the individual a pharmaceutical composition comprising a small molecule inhibitor of WRN such that the helicase activity of WRN is decreased in the proliferative cells. In some embodiments, the small molecule WRN inhibitor does not decrease the exonuclease activity of WRN in the proliferative cells. In some embodiments, the small molecule inhibitor has the formula:

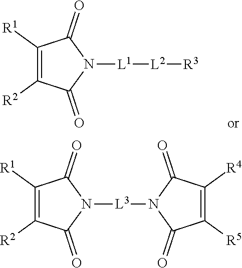

or a pharmaceutically acceptable salt thereof,

wherein:
L¹ is C₁₋₄ alkylene;
L² is O, S, OC(O), OSO₂, OC(O)O, or OC(O)NH;
L³ is C₁₋₈ alkylene;
R¹, R², R⁴, and R⁵ are each independently H, halogen, C₁₋₄ alkyl, or C₁₋₄ haloalkyl; and
R³ is H, C₁₋₆ alkyl, C₂₋₆ alkenyl, C₂₋₆ alkynyl, C₁₋₆ hydroxyalkyl, C₁₋₆ haloalkyl, C₆₋₁₂ aryl,
   or C₆₋₁₂ aryl-C₁₋₄ alkyl, each of which is optionally substituted with halogen, C₁₋₄ alkyl, or C₁₋₄ haloalkyl.

In some embodiments, L¹ is C₁₋₄ alkylene. The C₁₋₄ alkylene of L¹ can be methylene (CH₂), ethylene, propylene, isopropylene, butylene, isobutylene, or sec-butylene. In some embodiments, L¹ is CH₂.

In some embodiments, L² is OC(O).

In some embodiments, L³ is C₁₋₈ alkylene. In some embodiments, L³ is C₁₋₆ alkylene. The C₁₋₈ alkylene of L³ can be methylene, ethylene, propylene, isopropylene, butylene, isobutylene, sec-butylene, pentenylene, 2,2-dimethylpropylene (CH₂C(CH₃)₂CH₂), pentylene, hexylene, heptylene, or octylene. In some embodiments, L³ is CH₂C(CH₃)₂CH₂.

In some embodiments, R¹, R², R⁴, and R⁵ are each independently H or halogen. In some embodiments, R¹ and R² are each independently H or halogen. In some embodiments, R¹, R², R⁴, and R⁵ are each H. In some embodiments, R¹ and R² are each H. In some embodiments, R¹, R², R⁴, and R⁵ are each halogen. In some embodiments, R¹ and R² are each halogen. Halogen can be F, Cl, Br, or I. In some embodiments, R¹, R², R⁴, and R⁵ are each Cl. In some embodiments, R¹ and R² are each Cl.

In some embodiments, R³ is H, C₁₋₆ alkyl, C₂₋₆ alkenyl, C₂₋₆ alkynyl, C₁₋₆ hydroxyalkyl, C₁₋₆ haloalkyl, phenyl, or benzyl, each of which is optionally substituted with halogen, C₁₋₄ alkyl, or C₁₋₄ haloalkyl. In some embodiments, R³ is C₁₋₆ alkyl. The C₁₋₆ alkyl of R³ can be methyl, ethyl, propyl, isopropyl, butyl, isobutyl, sec-butyl, tert-butyl, pentyl, isopentyl, or hexyl. In some embodiments, R³ is ethyl.

In some embodiments, the small molecule inhibitor is selected from the group consisting of NSC 617145 and NSC 19630. In some embodiments, the small molecule inhibitor is other than NSC 617145, NSC 19630, and ML-216.

In some embodiments, provided herein is a method for treating a proliferative disease in an individual in need thereof, the proliferative disease being characterized by proliferative cells having an MSI (or MSI-H), comprising administering to the individual a pharmaceutical composition comprising an ADC comprising an antibody conjugated to a WRN inhibitor, such that the helicase activity of WRN is decreased in the proliferative cells. In some embodiments, the ADC does not decrease the exonuclease activity of WRN in the proliferative cells. In some embodiments, the antibody in the ADC targets the ADC to the proliferative cells.

In another aspect, provided herein is a method for treating a proliferative disease in an individual in need thereof, the proliferative disease being characterized by proliferative cells having an MSI (or MSI-H), comprising administering to the individual a pharmaceutical composition comprising a nuclease capable of modifying the genomes of the proliferative cells such that the helicase activity of WRN in the proliferative cells is decreased, or a nucleic acid encoding the nuclease. In some embodiments, WRN exonuclease activity in the proliferative cells is not decreased. In some embodiments, the nuclease is a transcription activator-like effector nuclease (TALEN) or zinc-finger nucleases (ZFN) targeting a genomic sequence within or near an endogenous WRN gene locus. In some embodiments, the nuclease is an RNA-guided endonuclease (RGEN), and the method further comprises administering to the individual a gRNA comprising a spacer sequence that is complementary to a genomic sequence within or near an endogenous WRN gene locus, or a nucleic acid encoding the gRNA. In some embodiments, the genomes of the proliferative cells are modified by non-homologous end joining (NHEJ).

In some embodiments, provided herein is a method for treating a proliferative disease in an individual in need thereof, the proliferative disease being characterized by proliferative cells having an MSI (or MSI-H), comprising administering to the individual a pharmaceutical composition comprising a TALEN or ZFN targeting a genomic sequence within or near an endogenous WRN gene locus, such that the helicase activity of WRN in the proliferative cells is decreased, or a nucleic acid encoding the nuclease. In some embodiments, WRN exonuclease activity in the proliferative cells is not decreased.

In some embodiments, provided herein is a method for treating a proliferative disease in an individual in need thereof, the proliferative disease being characterized by proliferative cells having an MSI (or MSI-H), comprising administering to the individual a pharmaceutical composition comprising a) a gRNA comprising a spacer sequence that is complementary to a genomic sequence within or near an endogenous WRN gene locus, or a nucleic acid encoding the gRNA; and b) an RNA-guided endonuclease (RGEN), or a nucleic acid encoding the RGEN, such that the helicase activity of WRN in the proliferative cells is decreased. In some embodiments, WRN exonuclease activity in the proliferative cells is not decreased. In some embodiments, the nucleic acid encoding the gRNA is contained in an Adeno Associated Virus (AAV) vector and/or the nucleic acid encoding the RGEN is contained in an AAV vector. In some embodiments, the spacer sequence is complementary to a genomic sequence within a coding region of the endogenous WRN gene. In some embodiments, the genomes of the proliferative cells are modified by non-homologous end joining (NHEJ).

In some embodiments, according to any of the methods described herein comprising administering to an individual a gRNA or nucleic acid encoding the gRNA and an RGEN or nucleic acid encoding the RGEN, the method further comprises administering to the individual a donor template comprising a donor nucleic acid, wherein the donor template is configured such that the donor nucleic acid is capable of being inserted into the WRN gene locus by homologous recombination. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in the WRN helicase domain that decreases WRN helicase activity. In some embodiments, the donor nucleic acid encodes a deletion in the WRN helicase domain that reduces or eliminates WRN helicase activity. In some embodiments, the donor template is contained in an AAV vector.

In some embodiments, according to any of the methods described herein comprising administering to an individual a gRNA or nucleic acid encoding the gRNA and an RGEN or nucleic acid encoding the RGEN, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof. In some embodiments, the RGEN is Cas9.

In some embodiments, according to any of the methods described herein comprising administering to an individual a gRNA or nucleic acid encoding the gRNA and an RGEN or nucleic acid encoding the RGEN, the method comprises administering to the individual a nucleic acid encoding the RGEN. In some embodiments, the nucleic acid encoding the RGEN is a ribonucleic acid (RNA), such as an mRNA. In some embodiments, the method comprises administering to the individual the gRNA. In some embodiments, the RNA encoding the RGEN is linked to the gRNA via a covalent bond. In some embodiments, the nucleic acid encoding the RGEN is a deoxyribonucleic acid (DNA), such as a DNA plasmid. In some embodiments, the nucleic acid encoding the RGEN is formulated in a liposome or lipid nanoparticle. In some embodiments, the liposome or lipid nanoparticle encapsulates the gRNA.

In some embodiments, according to any of the methods described herein comprising administering to an individual a gRNA or nucleic acid encoding the gRNA and an RGEN or nucleic acid encoding the RGEN, the method comprises administering to the individual the RGEN. In some embodiments, the method comprises administering to the individual the gRNA. In some embodiments, the RGEN is pre-complexed with the gRNA, forming a Ribonucleoprotein (RNP) complex.

In another aspect, provided herein is a method for treating a proliferative disease in an individual in need thereof, the proliferative disease being characterized by proliferative cells having an MSI, comprising administering to the individual a pharmaceutical composition comprising a nucleic acid construct comprising a donor nucleic acid, wherein the nucleic acid construct is configured such that insertion of the donor nucleic acid into the genomes of the proliferative cells by homologous recombination decreases the helicase activity of WRN in the proliferative cells. In some embodiments, WRN exonuclease activity is not decreased in the proliferative cells. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in the WRN helicase domain that decreases WRN helicase activity. In some embodiments, the donor nucleic acid encodes a deletion in the WRN helicase domain that reduces or eliminates WRN helicase activity. In some embodiments, the nucleic acid construct is an AAV vector. In some embodiments, the AAV vector comprises two homology arms having sequences identical or substantially homologous (such at least about any of 90%, 95%, 96%, 97%, 98%, or 99% homologous) to regions of the endogenous WRN gene. In some embodiments, the AAV vector is an AAV clade F vector.

In some embodiments, according to any of the methods described herein for treating a proliferative disease, the proliferative cells comprise one or more (such as any of 2, 3, 4, or 5) MSI markers selected from the group consisting of BAT25, BAT26, D2S123, D5S346, and D17S250. In some embodiments, the proliferative cells comprise one MSI marker selected from the group consisting of BAT25, BAT26, D2S123, D5S346, and D17S250. In some embodiments, the proliferative cells comprise two MSI markers selected from the group consisting of BAT25, BAT26, D2S123, D5S346, and D17S250. In some embodiments, the proliferative cells comprise three MSI markers selected from the group consisting of BAT25, BAT26, D2S123, D5S346, and D17S250. In some embodiments, the proliferative cells comprise four MSI markers selected from the group consisting of BAT25, BAT26, D2S123, D5S346, and D17S250. In some embodiments, the proliferative cells comprise five MSI markers selected from the group consisting of BAT25, BAT26, D2S123, D5S346, and D17S250. In some embodiments, the method further comprises determining the presence of the one or more MSI markers in a population of proliferative cells from the individual to identify the presence of MSI in the proliferative cells. In some embodiments, the step of determining the presence of the one or more MSI markers is carried out prior to administering the pharmaceutical composition.

In some embodiments, according to any of the methods described herein for treating a proliferative disease, the proliferative cells comprise one or more mutations that impair DNA mismatch repair. In some embodiments, the one or more mutations comprise a mutation in a MutS homolog and/or a mutation in a MutL homolog. In some embodiments, the MutS homolog is selected from the group consisting of MSH2, MSH3, and MSH6. In some embodiments, the MutL homolog is selected from the group consisting of MLH1, MLH3, PMS1, and PMS2. In some embodiments, the proliferative cells comprise a mutation in MLH1. In some embodiments, the proliferative cells comprise a mutation in MSH2. In some embodiments, the proliferative cells comprise a mutation in PMS2. In some embodiments, the proliferative cells comprise a mutation in MLH1 and a mutation in MSH2. In some embodiments, the proliferative cells comprise a mutation in MLH1 and a mutation in PMS2. In some embodiments, the proliferative cell comprises a mutation in at least one MMR protein selected from MLH1, MSH2, MSH6, and PMS2.

In some embodiments, the method further comprises determining the presence of the one or more mutations in a population of proliferative cells from the individual to identify the presence of the one or more mutations in the proliferative cells. In some embodiments, the step of determining the presence of the one or more mutations is carried out prior to administering the pharmaceutical composition.

In some embodiments, according to any of the methods described herein for treating a proliferative disease, the proliferative cells comprise one or more markers of DNA damage. In some embodiments, the one or more markers of DNA damage are selected from the group consisting of high p21 expression and high γH2AX expression. In some embodiments, the proliferative cells comprise high p21 expression. In some embodiments, the proliferative cells comprise high γH2AX expression. In some embodiments, the proliferative cells comprise high p21 expression and high γH2AX expression. In some embodiments, the method further comprises determining the presence of the one or more markers of DNA damage in a population of proliferative cells from the individual to identify the presence of the one or more markers of DNA damage in the proliferative cells. In some embodiments, the step of determining the presence of the one or more markers of DNA damage is carried out prior to administering the pharmaceutical composition.

In some embodiments, according to any of the methods described herein for treating a proliferative disease, the amount of proliferative cells in the individual is decreased as compared to a corresponding individual that does not receive administration of the pharmaceutical composition.

In some embodiments, according to any of the methods described herein for treating a proliferative disease, the rate of proliferation of the proliferative cells is decreased as compared to a corresponding individual that does not receive administration of the pharmaceutical composition.

In some embodiments, according to any of the methods described herein for treating a proliferative disease, at least some of the proliferative cells are induced to undergo cell cycle arrest. In some embodiments, at least some of the proliferative cells are induced to be arrested in G1. In some embodiments, at least some of the proliferative cells are induced to be arrested in G2.

In some embodiments, according to any of the methods described herein for treating a proliferative disease, at least some of the proliferative cells are induced to undergo apoptosis. In some embodiments, at least some of the proliferative cells are induced to have increased caspase activity.

In some embodiments, according to any of the methods described herein for treating a proliferative disease, the individual is a mammal. In some embodiments, the mammal is a primate, such as a human. In some embodiments, according to any of the methods described herein for treating a proliferative disease, the individual is a veterinary animal.

In some embodiments, according to any of the methods described herein for treating a proliferative disease, the proliferative disease is cancer. In some embodiments, the cancer includes, without limitation, colon cancer, gastric cancer, endometrium cancer, ovarian cancer, hepatobiliary tract cancer, urinary tract cancer, brain cancer, and skin cancer.

In some embodiments, according to any of the methods described herein for treating a proliferative disease, the method further comprises administering to the individual a conventional therapy for the proliferative disease. In some embodiments, the conventional therapy is an anti-PD-1 therapy.

In another aspect, provided herein is a method for treating a disease or condition in an individual in need thereof, wherein the disease or condition is characterized by a cell having an MSI, the method comprising administering to the individual a pharmaceutical agent effective for decreasing the expression and/or activity of WRN in the cell. In some embodiments, the pharmaceutical agent is an inhibitor of WRN. In some embodiments, that pharmaceutical agent is a small molecule inhibitor. In some embodiments, the pharmaceutical agent is an ADC comprising an antibody conjugated to a WRN inhibitor. In some embodiments, the pharmaceutical agent is a nuclease capable of modifying the genomes of the proliferative cells such that (i) the expression of WRN in the proliferative cells is decreased or (ii) the activity of WRN (e.g., the helicase activity of WRN) in the proliferative cells is decreased, or a nucleic acid encoding the nuclease. In some embodiments, the pharmaceutical agent is a nucleic acid construct comprising a donor nucleic acid, wherein the nucleic acid construct is configured such that insertion of the donor nucleic acid into the genomes of the proliferative cells by homologous recombination (i) decreases the expression of WRN in the proliferative cells, or (ii) decreases the activity of WRN in the proliferative cells.

In another aspect, provided herein is a method for treating a proliferative disease in an individual in need thereof, the proliferative disease being characterized by proliferative cells having an MSI, comprising administering to the individual a pharmaceutical composition comprising an inhibitory nucleic acid targeting WRN mRNA, or a nucleic acid encoding the inhibitory nucleic acid, such that the expression of WRN is decreased in the proliferative cells. In some embodiments, the inhibitory nucleic acid comprises a short interfering RNA (siRNA), a microRNA (miRNA), or an antisense oligonucleotide.

In another aspect, provided herein is a method for treating a proliferative disease in an individual in need thereof, the proliferative disease being characterized by proliferative cells having an MSI, comprising administering to the individual a pharmaceutical composition comprising a proteolysis targeting chimera (PROTAC) that targets WRN for ubiquitination and proteolytic degradation. In some embodiments, the PROTAC comprises an E3 ubiquitin ligase ligand coupled via a linker to a WRN ligand.

### Methods of Diagnosis

In another aspect, provided herein is a method for predicting if an individual diagnosed with a proliferative disease is likely to respond to a therapy comprising administering to the individual a pharmaceutical agent effective for decreasing WRN helicase activity, the method comprising determining the presence of an MSI, or a marker associated with an MSI (or MSI-H), in a population of proliferative cells from the individual, and determining a likelihood that the individual will respond to the therapy based on the determination of the presence of MSI, or a marker associated with MSI, in the population of proliferative cells. In some embodiments, the population of proliferative cells comprises cancer cells. In some embodiments, the population of proliferative cells comprises circulating tumor cells. In some embodiments, the individual is a mammal, such as a primate, e.g., a human. In some embodiments, the individual is a human. In some embodiments, the individual is a veterinary animal.

In another aspect, provided herein is a method for predicting if an individual diagnosed with a proliferative disease is likely to respond to a therapy comprising administering to the individual a pharmaceutical agent effective for decreasing WRN helicase activity, the method comprising determining the presence of an MSI in a population of proliferative cells from the individual, and determining a likelihood that the individual will respond to the therapy based on the determination of the presence of MSI in the population of proliferative cells. In some embodiments, the determination of the presence of MSI in the population of proliferative cells comprises determining the presence of one or more MSI markers selected from the group consisting of BAT25, BAT26, D2S123, D5S346, and D17S250. In some embodiments, the individual is predicted to respond to the therapy if the amount of cells in the population of proliferative cells determined to have at least one of the MSI markers is above a pre-determined threshold for the proliferative disease. In some embodiments, the individual is predicted not to respond to the therapy if (a) the amount of cells in the population of proliferative cells determined to have at least one of the MSI markers is below a pre-determined threshold for the proliferative disease; or (b) the population of proliferative cells is determined to have none of the MSI markers. In some embodiments, the proliferative disease is a cancer. In some embodiments, the cancer includes, without limitation, colon cancer, gastric cancer, endometrium cancer, ovarian cancer, hepatobiliary tract cancer, urinary tract cancer, brain cancer, and skin cancer. In some embodiments, the individual is a mammal, such as a primate, e.g., a human. In some embodiments, the individual is a human. In some embodiments, the individual is a veterinary animal.

In another aspect, provided herein is a method for predicting if an individual diagnosed with a proliferative disease is likely to respond to a therapy comprising administering to the individual a pharmaceutical agent effective for decreasing WRN helicase activity, the method comprising determining the presence of a marker associated with an MSI in a population of proliferative cells from the individual, and determining a likelihood that the individual will respond to the therapy based on the determination of the presence of a marker associated with MSI in the population of proliferative cells. In some embodiments, the determination of the presence of a marker associated with MSI in the population of proliferative cells comprises determining the presence of a mutation that impairs DNA mismatch repair. In some embodiments, the mutation comprises a mutation in a MutS homolog and/or a mutation in a MutL homolog. In some embodiments, the MutS homolog is selected from the group consisting of MSH2, MSH3, and MSH6, and the MutL homolog is selected from the group consisting of MLH1, MLH3, PMS1, and PMS2. In some embodiments, the mutation comprises a mutation in MLH1, MSH2, and/or PMS2. In some embodiments, the mutation comprises a mutation in MLH1 and MSH2. In some embodiments, the mutation comprises a mutation in MLH1 and PMS2. In some embodiments, the mutation comprises a mutation in at least one MMR proteins selected from MLH1, MSH2, MSH6, and PMS2. In some embodiments, the determination of the presence of a marker associated with MSI in the population of proliferative cells comprises determining the presence of one or more markers of DNA damage. In some embodiments, the one or more markers of DNA damage are selected from the group consisting of high p21 expression and high γH2AX expression. In some embodiments, the individual is predicted to respond to the therapy if the amount of cells in the population of proliferative cells determined to have (i) at least one mutation that impairs DNA mismatch repair and/or (ii) at least one marker of DNA damage is above a pre-determined threshold for the proliferative disease. In some embodiments, the at least one mutation that impairs DNA mismatch repair comprises a mutation in MLH1, MSH2, and/or PMS2, and the at least one marker of DNA damage comprises high p21 expression and/or high γH2AX expression. In some embodiments, the individual is predicted not to respond to the therapy if (a) the amount of cells in the population of proliferative cells determined to have (i) at least one mutation that impairs DNA mismatch repair and/or (ii) at least one marker of DNA damage is below a pre-determined threshold for the proliferative disease; or (b) the population of proliferative cells is determined to have no mutations that impair DNA mismatch repair and no DNA damage markers. In some embodiments, the proliferative disease is a cancer. In some embodiments, the cancer includes, without limitation, colon cancer, gastric cancer, endometrium cancer, ovarian cancer, hepatobiliary tract cancer, urinary tract cancer, brain cancer, and skin cancer. In some embodiments, the individual is a mammal, such as a primate, e.g., a human. In some embodiments, the individual is a human. In some embodiments, the individual is a veterinary animal.

In another aspect, provided herein is a method for detecting a microsatellite instability (MSI) (or MSI-H) and the helicase activity of WRN in an individual diagnosed with or thought to have a proliferative disease, the method comprising: (a) contacting a biological sample from the individual with one or more reagents for detecting the presence of an MSI and the helicase activity of WRN; and (b) detecting (i) the presence of an MSI; and (ii) the helicase activity of WRN. In some embodiments, the reagent for detecting the presence of MSI in a biological sample comprises a reagent for detecting the presence of one or more MSI markers selected from the group consisting of BAT25, BAT26, D2S123, D5S346, and D17S250. In some embodiments, the proliferative disease is a cancer. In some embodiments, the cancer includes, without limitation, colon cancer, gastric cancer, endometrium cancer, ovarian cancer, hepatobiliary tract cancer, urinary tract cancer, brain cancer, and skin cancer. In some embodiments, the biological sample comprises cancer cells. In some embodiments, the biological sample comprises circulating tumor cells. In some embodiments, the individual is a mammal, such as a primate, e.g., a human. In some embodiments, the individual is a human. In some embodiments, the individual is a veterinary animal.

In another aspect, provided herein is a method for detecting a marker associated with an MSI (or MSI-H) and the helicase activity of WRN in an individual diagnosed with or thought to have a proliferative disease, the method comprising: (a) contacting a biological sample from the individual with one or more reagents for detecting the presence of a marker associated with an MSI and the helicase activity of WRN; and (b) detecting (i) the presence of the marker associated with an MSI; and (ii) the helicase activity of WRN. In some embodiments, the reagent for detecting the presence of a marker associated with an MSI in a biological sample comprises a reagent for detecting the presence of (i) one or more mutations that impair DNA mismatch repair and/or (ii) one or more markers of DNA damage. In some embodiments, the one or more mutations that impair DNA mismatch repair comprise a mutation in a MutS homolog and/or a mutation in a MutL homolog. In some embodiments, the MutS homolog is selected from the group consisting of MSH2, MSH3, and MSH6, and the MutL homolog is selected from the group consisting of MLH1, MLH3, PMS1, and PMS2. In some embodiments, the one or more mutations comprise a mutation in MLH1, MSH2, and/or PMS2. In some embodiments, the one or more mutations comprise a mutation in MLH1 and MSH2. In some embodiments, the one or more mutations comprise a mutation in MLH1 and PMS2. In some embodiments, the mutation comprises a mutation in at least one MMR proteins selected from MLH1, MSH2, MSH6, and PMS2. In some embodiments, the one or more markers of DNA damage are selected from the group consisting of high p21 expression and high γH2AX expression. In some embodiments, the proliferative disease is a cancer. In some embodiments, the cancer includes, without limitation, colon cancer, gastric cancer, endometrium cancer, ovarian cancer, hepatobiliary tract cancer, urinary tract cancer, brain cancer, and skin cancer. In some embodiments, the biological sample comprises cancer cells. In some embodiments, the biological sample comprises circulating tumor cells. In some embodiments, the individual is a mammal, such as a primate, e.g., a human. In some embodiments, the individual is a human. In some embodiments, the individual is a veterinary animal.

### Compositions

In one aspect, provided herein is a composition comprising (a) a gRNA comprising a spacer sequence complementary to a genomic sequence within or near an endogenous WRN gene locus, or a nucleic acid encoding the gRNA; and b) an RNA-guided endonuclease (RGEN), or a nucleic acid encoding the RGEN, wherein the components of the composition are configured such that delivery of the composition into a cell is capable of decreasing the helicase activity of WRN in the cell. In some embodiments, delivery of the composition into a cell does not decrease WRN exonuclease activity in the cell. In some embodiments, the nucleic acid encoding the gRNA is contained in an Adeno Associated Virus (AAV) vector and/or the nucleic acid encoding the RGEN is contained in an AAV vector. In some embodiments, the spacer sequence is complementary to a genomic sequence within a coding region of the endogenous WRN gene. In some embodiments, the composition further comprises a donor template comprising a donor nucleic acid, wherein the donor template is configured such that the donor nucleic acid is capable of being inserted into the WRN gene locus by homologous recombination. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in the WRN helicase domain that decreases WRN helicase activity. In some embodiments, the donor nucleic acid encodes a deletion in the WRN helicase domain that reduces or eliminates WRN helicase activity. In some embodiments, the donor template is contained in an AAV vector.

In some embodiments, according to any of the compositions described herein comprising a gRNA or nucleic acid encoding the gRNA and an RGEN or nucleic acid encoding the RGEN, the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof. In some embodiments, the RGEN is Cas9.

In some embodiments, according to any of the compositions described herein comprising a gRNA or nucleic acid encoding the gRNA and an RGEN or nucleic acid encoding the RGEN, the composition comprises a nucleic acid encoding the RGEN. In some embodiments, the nucleic acid encoding the RGEN is a ribonucleic acid (RNA), such as an mRNA. In some embodiments, the composition comprises the gRNA. In some embodiments, the RNA encoding the RGEN is linked to the gRNA via a covalent bond. In some embodiments, the nucleic acid encoding the RGEN is a deoxyribonucleic acid (DNA), such as a DNA plasmid. In some embodiments, the nucleic acid encoding the RGEN is formulated in a liposome or lipid nanoparticle. In some embodiments, the liposome or lipid nanoparticle encapsulates the gRNA.

In some embodiments, according to any of the compositions described herein comprising a gRNA or nucleic acid encoding the gRNA and an RGEN or nucleic acid encoding the RGEN, the composition comprises the RGEN. In some embodiments, the composition comprises the gRNA. In some embodiments, the RGEN is pre-complexed with the gRNA, forming a Ribonucleoprotein (RNP) complex.

In another aspect, provided herein is a composition comprising a nucleic acid construct comprising a donor nucleic acid, wherein the nucleic acid construct is configured such that insertion of the donor nucleic acid into the genome of a proliferative cell by homologous recombination decreases the helicase activity of WRN in the proliferative cell. In some embodiments, the donor nucleic acid encodes one or more STOP codons, and the donor template is configured such that the donor nucleic acid is inserted into a coding region of the WRN gene. In some embodiments, the donor nucleic acid encodes three STOP codons in each of the 3 translation frames present in succession. In some embodiments, the donor nucleic acid encodes a mutation in the WRN helicase domain that decreases WRN helicase activity. In some embodiments, the donor nucleic acid encodes a deletion in the WRN helicase domain that reduces or eliminates WRN helicase activity. In some embodiments, the nucleic acid construct is an AAV vector. In some embodiments, the AAV vector comprises two homology arms having sequences identical or substantially homologous (such at least about any of 90%, 95%, 96%, 97%, 98%, or 99% homologous) to regions of the endogenous WRN gene. In some embodiments, the AAV vector is an AAV clade F vector.

### Nucleic Acids

### Genome-Targeting Nucleic Acid or Guide RNA

The present disclosure provides a genome-targeting nucleic acid that can direct the activities of an associated polypeptide (e.g., a site-directed polypeptide or DNA endonuclease) to a specific target sequence within a target nucleic acid. In some embodiments, the genome-targeting nucleic acid is an RNA. A genome-targeting RNA is referred to as a “guide RNA” or “gRNA” herein. A guide RNA has at least a spacer sequence that hybridizes to a target nucleic acid sequence of interest and a CRISPR repeat sequence. In Type II systems, the gRNA also has a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-directed polypeptide such that the guide RNA and site-direct polypeptide form a complex. The genome-targeting nucleic acid provides target specificity to the complex by virtue of its association with the site-directed polypeptide. The genome-targeting nucleic acid thus directs the activity of the site-directed polypeptide.

In some embodiments, the genome-targeting nucleic acid is a double-molecule guide RNA. In some embodiments, the genome-targeting nucleic acid is a single-molecule guide RNA. A double-molecule guide RNA has two strands of RNA. The first strand has in the 5′ to 3′ direction, an optional spacer extension sequence, a spacer sequence and a minimum CRISPR repeat sequence. The second strand has a minimum tracrRNA sequence (complementary to the minimum CRISPR repeat sequence), a 3′ tracrRNA sequence and an optional tracrRNA extension sequence. A single-molecule guide RNA (sgRNA) in a Type II system has, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may have elements that contribute additional functionality (e.g., stability) to the guide RNA. The single-molecule guide linker links the minimum CRISPR repeat and the minimum tracrRNA sequence to form a hairpin structure. The optional tracrRNA extension has one or more hairpins. A single-molecule guide RNA (sgRNA) in a Type V system has, in the 5′ to 3′ direction, a minimum CRISPR repeat sequence and a spacer sequence.

Exemplary genome-targeting nucleic acids are described in WO2018002719.

### Donor DNA or Donor Template

Site-directed polypeptides, such as a DNA endonuclease, can introduce double-strand breaks or single-strand breaks in nucleic acids, e.g., genomic DNA. The double-strand break can stimulate a cell's endogenous DNA-repair pathways (e.g., homology-dependent repair (HDR) or non-homologous end joining or alternative non-homologous end joining (A-NHEJ) or microhomology-mediated end joining (MMEJ). NHEJ can repair cleaved target nucleic acid without the need for a homologous template. This can sometimes result in small deletions or insertions (indels) in the target nucleic acid at the site of cleavage, and can lead to disruption or alteration of gene expression. HDR, which is also known as homologous recombination (HR) can occur when a homologous repair template, or donor, is available.

The homologous donor template has sequences that are homologous to sequences flanking the target nucleic acid cleavage site. The sister chromatid is generally used by the cell as the repair template. However, for the purposes of genome editing, the repair template is often supplied as an exogenous nucleic acid, such as a plasmid, duplex oligonucleotide, single-strand oligonucleotide, double-stranded oligonucleotide, or viral nucleic acid. With exogenous donor templates, it is common to introduce an additional nucleic acid sequence (such as a transgene) or modification (such as a single or multiple base change or a deletion) between the flanking regions of homology so that the additional or altered nucleic acid sequence also becomes incorporated into the target locus. MMEJ results in a genetic outcome that is similar to NHEJ in that small deletions and insertions can occur at the cleavage site. MMEJ makes use of homologous sequences of a few base pairs flanking the cleavage site to drive a favored end-joining DNA repair outcome. In some instances, it can be possible to predict likely repair outcomes based on analysis of potential microhomologies in the nuclease target regions.

Thus, in some cases, homologous recombination is used to insert an exogenous polynucleotide sequence into the target nucleic acid cleavage site. An exogenous polynucleotide sequence is termed a donor polynucleotide (or donor or donor sequence or polynucleotide donor template) herein. In some embodiments, the donor polynucleotide, a portion of the donor polynucleotide, a copy of the donor polynucleotide, or a portion of a copy of the donor polynucleotide is inserted into the target nucleic acid cleavage site. In some embodiments, the donor polynucleotide is an exogenous polynucleotide sequence, i.e., a sequence that does not naturally occur at the target nucleic acid cleavage site.

When an exogenous DNA molecule is supplied in sufficient concentration inside the nucleus of a cell in which the double strand break occurs, the exogenous DNA can be inserted at the double strand break during the NHEJ repair process and thus become a permanent addition to the genome. These exogenous DNA molecules are referred to as donor templates in some embodiments. If the donor template contains a coding sequence for one or more system components described herein optionally together with relevant regulatory sequences such as promoters, enhancers, polyA sequences and/or splice acceptor sequences, the one or more system components can be expressed from the integrated nucleic acid in the genome resulting in permanent expression for the life of the cell. Moreover, the integrated nucleic acid of the donor DNA template can be transmitted to the daughter cells when the cell divides.

In the presence of sufficient concentrations of a donor DNA template that contains flanking DNA sequences with homology to the DNA sequence either side of the double strand break (referred to as homology arms), the donor DNA template can be integrated via the HDR pathway. The homology arms act as substrates for homologous recombination between the donor template and the sequences either side of the double strand break. This can result in an error free insertion of the donor template in which the sequences either side of the double strand break are not altered from that in the unmodified genome.

Supplied donors for editing by HDR vary markedly but generally contain the intended sequence with small or large flanking homology arms to allow annealing to the genomic DNA. The homology regions flanking the introduced genetic changes can be 30 bp or smaller, or as large as a multi-kilobase cassette that can contain promoters, cDNAs, etc. Both single-stranded and double-stranded oligonucleotide donors can be used. These oligonucleotides range in size from less than 100 nt to over many kb, though longer ssDNA can also be generated and used. Double-stranded donors are often used, including PCR amplicons, plasmids, and mini-circles. In general, it has been found that an AAV vector is a very effective means of delivery of a donor template, though the packaging limits for individual donors is <5 kb. Active transcription of the donor increased HDR three-fold, indicating the inclusion of promoter can increase conversion. Conversely, CpG methylation of the donor can decrease gene expression and HDR.

In some embodiments, the donor DNA can be supplied with the nuclease or independently by a variety of different methods, for example by transfection, nanoparticle, micro-injection, or viral transduction. A range of tethering options can be used to increase the availability of the donors for HDR in some embodiments. Examples include attaching the donor to the nuclease, attaching to DNA binding proteins that bind nearby, or attaching to proteins that are involved in DNA end binding or repair.

In addition to genome editing by NHEJ or HDR, site-specific gene insertions can be conducted that use both the NHEJ pathway and HR. A combination approach can be applicable in certain settings, possibly including intron/exon borders. NHEJ can prove effective for ligation in the intron, while the error-free HDR can be better suited in the coding region.

### Nucleic Acid Encoding a Site-Directed Polypeptide or DNA Endonuclease

In some embodiments, the methods of genome edition and compositions therefore can use a nucleic acid sequence encoding a site-directed polypeptide or DNA endonuclease. The nucleic acid sequence encoding the site-directed polypeptide can be DNA or RNA. If the nucleic acid sequence encoding the site-directed polypeptide is RNA, it can be covalently linked to a gRNA sequence or exist as a separate sequence. In some embodiments, a peptide sequence of the site-directed polypeptide or DNA endonuclease can be used instead of the nucleic acid sequence thereof.

Vectors

In another aspect, the present disclosure provides a nucleic acid having a nucleotide sequence encoding a genome-targeting nucleic acid of the disclosure, a site-directed polypeptide of the disclosure, and/or any nucleic acid or proteinaceous molecule necessary to carry out the embodiments of the methods of the disclosure. In some embodiments, such a nucleic acid is a vector (e.g., a recombinant expression vector).

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Additional vectors contemplated for eukaryotic target cells include, but are not limited to, the vectors pCTx-1, pCTx-2, and pCTx-3. Other vectors can be used so long as they are compatible with the host cell.

In some embodiments, a vector has one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. In some embodiments, the vector is a self-inactivating vector that either inactivates the viral sequences or the components of the CRISPR machinery or other elements.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct having the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with Cas endonuclease, various promoters such as RNA polymerase III promoters, including for example U6 and H1, can be advantageous. Descriptions of and parameters for enhancing the use of such promoters are known in art, and additional information and approaches are regularly being described; see, e.g., Ma, H. et al., Molecular Therapy—Nucleic Acids 3, e161 (2014) doi:10.1038/mtna.2014.12.

The expression vector can also contain a ribosome binding site for translation initiation and a transcription terminator. The expression vector can also include appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

In some embodiments, a promoter is an inducible promoter (e.g., a heat shock promoter, tetracycline-regulated promoter, steroid-regulated promoter, metal-regulated promoter, estrogen receptor-regulated promoter, etc.). In some embodiments, a promoter is a constitutive promoter (e.g., CMV promoter, UBC promoter). In some embodiments, the promoter is a spatially restricted and/or temporally restricted promoter (e.g., a tissue specific promoter, a cell type specific promoter, etc.). In some embodiments, a vector does not have a promoter for at least one gene to be expressed in a host cell if the gene is going to be expressed, after it is inserted into a genome, under an endogenous promoter present in the genome.

### Site-Directed Polypeptide or DNA Endonuclease

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The process of integrating non-native nucleic acid into genomic DNA is an example of genome editing.

A site-directed polypeptide is a nuclease used in genome editing to cleave DNA. The site-directed polypeptide can be administered to a cell or a patient as either: one or more polypeptides, or one or more nucleic acids encoding the polypeptide.

In the context of a CRISPR/Cas or CRISPR/Cpf1 system, the site-directed polypeptide can bind to a guide RNA that, in turn, specifies the site in the target DNA to which the polypeptide is directed. In embodiments of CRISPR/Cas or CRISPR/Cpf1 systems herein, the site-directed polypeptide is an endonuclease, such as a DNA endonuclease. Such an RNA-guided site-directed polypeptide is also referred to herein as an RNA-guided endonuclease, or RGEN.

Exemplary site-directed polypeptides are described in WO2018002719.

### Target Sequence Selection

In some embodiments, shifts in the location of the 5′ boundary and/or the 3′ boundary relative to particular reference loci are used to facilitate or enhance particular applications of gene editing, which depend in part on the endonuclease system selected for the editing, as further described and illustrated herein.

In a first, non-limiting aspect of such target sequence selection, many endonuclease systems have rules or criteria that guide the initial selection of potential target sites for cleavage, such as the requirement of a PAM sequence motif in a particular position adjacent to the DNA cleavage sites in the case of CRISPR Type II or Type V endonucleases.

In another, non-limiting aspect of target sequence selection or optimization, the frequency of “off-target” activity for a particular combination of target sequence and gene editing endonuclease (i.e. the frequency of DSBs occurring at sites other than the selected target sequence) is assessed relative to the frequency of on-target activity. In some cases, cells that have been correctly edited at the desired locus can have a selective advantage relative to other cells. Illustrative, but non-limiting, examples of a selective advantage include the acquisition of attributes such as enhanced rates of replication, persistence, resistance to certain conditions, enhanced rates of successful engraftment or persistence in vivo following introduction into a patient, and other attributes associated with the maintenance or increased numbers or viability of such cells. In other cases, cells that have been correctly edited at the desired locus can be positively selected for by one or more screening methods used to identify, sort or otherwise select for cells that have been correctly edited. Both selective advantage and directed selection methods can take advantage of the phenotype associated with the correction. In some embodiments, cells can be edited two or more times in order to create a second modification that creates a new phenotype that is used to select or purify the intended population of cells. Such a second modification could be created by adding a second gRNA for a selectable or screenable marker. In some cases, cells can be correctly edited at the desired locus using a DNA fragment that contains the cDNA and also a selectable marker.

In embodiments, whether any selective advantage is applicable or any directed selection is to be applied in a particular case, target sequence selection is also guided by consideration of off-target frequencies in order to enhance the effectiveness of the application and/or reduce the potential for undesired alterations at sites other than the desired target. As described further and illustrated herein and in the art, the occurrence of off-target activity is influenced by a number of factors including similarities and dissimilarities between the target site and various off-target sites, as well as the particular endonuclease used. Bioinformatics tools are available that assist in the prediction of off-target activity, and frequently such tools can also be used to identify the most likely sites of off-target activity, which can then be assessed in experimental settings to evaluate relative frequencies of off-target to on-target activity, thereby allowing the selection of sequences that have higher relative on-target activities. Illustrative examples of such techniques are provided herein, and others are known in the art.

Another aspect of target sequence selection relates to homologous recombination events. Sequences sharing regions of homology can serve as focal points for homologous recombination events that result in deletion of intervening sequences. Such recombination events occur during the normal course of replication of chromosomes and other DNA sequences, and also at other times when DNA sequences are being synthesized, such as in the case of repairs of double-strand breaks (DSBs), which occur on a regular basis during the normal cell replication cycle but can also be enhanced by the occurrence of various events (such as UV light and other inducers of DNA breakage) or the presence of certain agents (such as various chemical inducers). Many such inducers cause DSBs to occur indiscriminately in the genome, and DSBs are regularly being induced and repaired in normal cells. During repair, the original sequence can be reconstructed with complete fidelity, however, in some cases, small insertions or deletions (referred to as “indels”) are introduced at the DSB site.

DSBs can also be specifically induced at particular locations, as in the case of the endonucleases systems described herein, which can be used to cause directed or preferential gene modification events at selected chromosomal locations. The tendency for homologous sequences to be subject to recombination in the context of DNA repair (as well as replication) can be taken advantage of in a number of circumstances, and is the basis for one application of gene editing systems, such as CRISPR, in which homology directed repair is used to insert a sequence of interest, provided through use of a “donor” polynucleotide, into a desired chromosomal location.

Regions of homology between particular sequences, which can be small regions of “microhomology” that can have as few as ten base pairs or less, can also be used to bring about desired deletions. For example, a single DSB is introduced at a site that exhibits microhomology with a nearby sequence. During the normal course of repair of such DSB, a result that occurs with high frequency is the deletion of the intervening sequence as a result of recombination being facilitated by the DSB and concomitant cellular repair process.

In some circumstances, however, selecting target sequences within regions of homology can also give rise to much larger deletions, including gene fusions (when the deletions are in coding regions), which can or cannot be desired given the particular circumstances.

### Targeted Integration

In some embodiments, a method provided herein employs a step of integrating donor nucleic acid as described herein at a specific location in the genome of target cells (e.g., proliferative cells that are MSI-H), which is referred to as “targeted integration”. In some embodiments, targeted integration is enabled by using a sequence specific nuclease to generate a double stranded break in the genomic DNA.

The CRISPR-Cas system used in some embodiments has the advantage that a large number of genomic targets can be rapidly screened to identify an optimal CRISPR-Cas design. The CRISPR-Cas system uses a RNA molecule called a single guide RNA (sgRNA) that targets an associated Cas nuclease (for example the Cas9 nuclease) to a specific sequence in DNA. This targeting occurs by Watson-Crick based pairing between the sgRNA and the sequence of the genome within the approximately 20 bp targeting sequence of the sgRNA. Once bound at a target site the Cas nuclease cleaves both strands of the genomic DNA creating a double strand break. The only requirement for designing a sgRNA to target a specific DNA sequence is that the target sequence must contain a protospacer adjacent motif (PAM) sequence at the 3′ end of the sgRNA sequence that is complementary to the genomic sequence. In the case of the Cas9 nuclease the PAM sequence is NRG (where R is A or G and N is any base), or the more restricted PAM sequence NGG. Therefore, sgRNA molecules that target any region of the genome can be designed in silico by locating the 20 bp sequence adjacent to all PAM motifs. PAM motifs occur on average very 15 bp in the genome of eukaryotes. However, sgRNA designed by in silico methods will generate double strand breaks in cells with differing efficiencies and it is not possible to predict the cutting efficiencies of a series of sgRNA molecule using in silico methods. Because sgRNA can be rapidly synthesized in vitro this enables the rapid screening of all potential sgRNA sequences in a given genomic region to identify the sgRNA that results in the most efficient cutting. Typically when a series of sgRNA within a given genomic region are tested in cells a range of cleavage efficiencies between 0 and 90% is observed. In silico algorithms as well as laboratory experiments can also be used to determine the off-target potential of any given sgRNA. While a perfect match to the 20 bp recognition sequence of a sgRNA will primarily occur only once in most eukaryotic genomes there will be a number of additional sites in the genome with 1 or more base pair mismatches to the sgRNA. These sites can be cleaved at variable frequencies which are often not predictable based on the number or location of the mismatches. Cleavage at additional off-target sites that were not identified by the in silico analysis can also occur. Thus, screening a number of sgRNA in a relevant cell type to identify sgRNA that have the most favorable off-target profile is a critical component of selecting an optimal sgRNA for therapeutic use. A favorable off target profile will take into account not only the number of actual off-target sites and the frequency of cutting at these sites, but also the location in the genome of these sites. For example, off-target sites close to or within functionally important genes, particularly oncogenes or anti-oncogenes would be considered as less favorable than sites in intergenic regions with no known function. Thus, the identification of an optimal sgRNA cannot be predicted simply by in silico analysis of the genomic sequence of an organism but requires experimental testing. While in silico analysis can be helpful in narrowing down the number of guides to test it cannot predict guides that have high on target cutting or predict guides with low desirable off-target cutting. The ability of a given sgRNA to promote cleavage by a Cas enzyme can relate to the accessibility of that specific site in the genomic DNA which can be determined by the chromatin structure in that region. While the majority of the genomic DNA in a quiescent differentiated cell exists in highly condensed heterochromatin, regions that are actively transcribed exists in more open chromatin states that are known to be more accessible to large molecules such as proteins like the Cas protein. Even within actively transcribed genes some specific regions of the DNA are more accessible than others due to the presence or absence of bound transcription factors or other regulatory proteins. Predicting sites in the genome or within a specific genomic locus or region of a genomic locus is not possible and therefore would need to be determined experimentally in a relevant cell type. Once some sites are selected as potential sites for insertion, it can be possible to add some variations to such a site, e.g. by moving a few nucleotides upstream or downstream from the selected sites, with or without experimental tests.

### Nucleic Acid Modifications

In some embodiments, polynucleotides introduced into cells have one or more modifications that can be used independently or in combination, for example, to enhance activity, stability or specificity, alter delivery, reduce innate immune responses in host cells, or for other enhancements, as further described herein and known in the art.

In certain embodiments, modified polynucleotides are used in the CRISPR/Cas9/Cpf1 system, in which case the guide RNAs (either single-molecule guides or double-molecule guides) and/or a DNA or an RNA encoding a Cas or Cpf1 endonuclease introduced into a cell can be modified, as described below. Such modified polynucleotides can be used in the CRISPR/Cas9/Cpf1 system to edit any one or more genomic loci.

Using the CRISPR/Cas9/Cpf1 system for purposes of non-limiting illustrations of such uses, modifications of guide RNAs can be used to enhance the formation or stability of the CRISPR/Cas9/Cpf1 genome editing complex having guide RNAs, which can be single-molecule guides or double-molecule, and a Cas or Cpf1 endonuclease. Modifications of guide RNAs can also or alternatively be used to enhance the initiation, stability or kinetics of interactions between the genome editing complex with the target sequence in the genome, which can be used, for example, to enhance on-target activity. Modifications of guide RNAs can also or alternatively be used to enhance specificity, e.g., the relative rates of genome editing at the on-target site as compared to effects at other (off-target) sites.

Modifications can also or alternatively be used to increase the stability of a guide RNA, e.g., by increasing its resistance to degradation by ribonucleases (RNases) present in a cell, thereby causing its half-life in the cell to be increased. Modifications enhancing guide RNA half-life can be particularly useful in embodiments in which a Cas or Cpf1 endonuclease is introduced into the cell to be edited via an RNA that needs to be translated in order to generate endonuclease, because increasing the half-life of guide RNAs introduced at the same time as the RNA encoding the endonuclease can be used to increase the time that the guide RNAs and the encoded Cas or Cpf1 endonuclease co-exist in the cell.

Modifications can also or alternatively be used to decrease the likelihood or degree to which RNAs introduced into cells elicit innate immune responses. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses.

One or more types of modifications can also be made to RNAs encoding an endonuclease that are introduced into a cell, including, without limitation, modifications that enhance the stability of the RNA (such as by increasing its degradation by RNAses present in the cell), modifications that enhance translation of the resulting product (i.e. the endonuclease), and/or modifications that decrease the likelihood or degree to which the RNAs introduced into cells elicit innate immune responses.

Combinations of modifications, such as the foregoing and others, can likewise be used. In the case of CRISPR/Cas9/Cpf1, for example, one or more types of modifications can be made to guide RNAs (including those exemplified above), and/or one or more types of modifications can be made to RNAs encoding Cas endonuclease (including those exemplified above).

Exemplary modified nucleic acids are described in WO2018002719.

### Delivery

In some embodiments, any nucleic acid molecules used in the methods provided herein, e.g. a nucleic acid encoding a genome-targeting nucleic acid of the disclosure and/or a site-directed polypeptide are packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Exemplary delivery methods and reagents are described in WO2018002719.

Additional embodiments of the invention include embodiments 1-145 below:

Embodiment 1. A method for decreasing proliferation in a proliferative cell having a microsatellite instability (MSI), comprising decreasing the helicase activity of Werner syndrome ATP-dependent helicase (WRN) in the proliferative cell.

Embodiment 2. The method of embodiment 1, comprising delivering into the proliferative cell an inhibitor of WRN.

Embodiment 3. The method of embodiment 2, wherein the inhibitor of WRN is a small molecule inhibitor.

Embodiment 4. The method of embodiment 3, wherein the small molecule inhibitor has the formula:

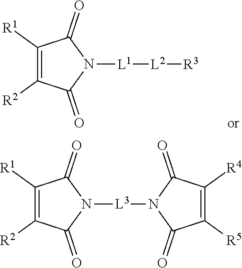

or a pharmaceutically acceptable salt thereof,

wherein
L¹ is C₁₋₄ alkylene;
L² is O, S, OC(O), OSO₂, OC(O)O, or OC(O)NH;
L³ is C₁₋₈ alkylene;
R¹, R², R⁴ and R⁵ are each independently H, halogen, C₁₋₄ alkyl, or C₁₋₄ haloalkyl; and
R³ is H, C₁₋₆ alkyl, C₂₋₆ alkenyl, C₂₋₆ alkynyl, C₁₋₆ hydroxyalkyl, C₁₋₆ haloalkyl, C₆₋₁₂ aryl, or C₆₋₁₂ aryl-C₁₋₄ alkyl, each of which is optionally substituted with halogen, C₁₋₄ alkyl, or C₁₋₄ haloalkyl.

Embodiment 5. The method of embodiment 2, wherein the inhibitor of WRN comprises an antibody drug conjugate (ADC) comprising an antibody conjugated to a WRN inhibitor.

Embodiment 6. The method of embodiment 1, comprising delivering into the proliferative cell an inhibitory nucleic acid targeting WRN mRNA, or a nucleic acid encoding the inhibitory nucleic acid.

Embodiment 7. The method of embodiment 6, wherein the inhibitory nucleic acid comprises a short interfering RNA (siRNA), a microRNA (miRNA), or an antisense oligonucleotide.

Embodiment 8. The method of embodiment 1, comprising delivering into the proliferative cell a nuclease capable of modifying the genome of the proliferative cell such that the helicase activity of WRN in the proliferative cell is decreased, or a nucleic acid encoding the nuclease.

Embodiment 9. The method of embodiment 8, wherein the nuclease is a transcription activator-like effector nuclease (TALEN) or zinc-finger nucleases (ZFN) targeting a genomic sequence within or near an endogenous WRN gene locus.

Embodiment 10. The method of embodiment 8, comprising delivering into the proliferative cell a) a gRNA comprising a spacer sequence that is complementary to a genomic sequence within or near an endogenous WRN gene locus, or a nucleic acid encoding the gRNA; and b) an RNA-guided endonuclease (RGEN), or a nucleic acid encoding the RGEN.

Embodiment 11. The method of embodiment 10, wherein the nucleic acid encoding the gRNA is contained in an Adeno Associated Virus (AAV) vector and/or the nucleic acid encoding the RGEN is contained in an AAV vector.

Embodiment 12. The method of embodiment 10 or 11, wherein the spacer sequence is complementary to a genomic sequence within a coding region of the endogenous WRN gene.

Embodiment 13. The method of any one of embodiments 10-12, wherein the genome of the proliferative cell is modified by non-homologous end joining (NHEJ).

Embodiment 14. The method of any one of embodiments 10-12, further comprising delivering into the proliferative cell a donor template comprising a donor nucleic acid, wherein the donor template is configured such that the donor nucleic acid is capable of being inserted into the WRN gene locus by homologous recombination.

Embodiment 15. The method of embodiment 14, wherein the donor nucleic acid encodes one or more STOP codons, and the donor template is configured such that the donor nucleic acid is inserted into a coding region of the WRN gene.

Embodiment 16. The method of embodiment 15, wherein the donor nucleic acid encodes three STOP codons in each of the 3 translation frames present in succession.

Embodiment 17. The method of embodiment 14, wherein the donor nucleic acid encodes a mutation in the WRN helicase domain that decreases WRN helicase activity.

Embodiment 18. The method of any one of embodiments 14-17, wherein the donor template is contained in an AAV vector.

Embodiment 19. The method of any one of embodiments 10-18, wherein the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof.

Embodiment 20. The method of embodiment 19, wherein the RGEN is Cas9.

Embodiment 21. The method of any one of embodiments 10-20, wherein the nucleic acid encoding the RGEN is a ribonucleic acid (RNA) sequence.

Embodiment 22. The method of embodiment 21, wherein the RNA sequence encoding the RGEN is linked to the gRNA via a covalent bond.

Embodiment 23. The method of any one of embodiments 10-20, wherein the nucleic acid encoding the RGEN is a deoxyribonucleic acid (DNA) sequence.

Embodiment 24. The method of any one of embodiments 10-23, wherein the nucleic acid encoding the RGEN is formulated in a liposome or lipid nanoparticle.

Embodiment 25. The method of embodiment 24, wherein the liposome or lipid nanoparticle encapsulates the gRNA.

Embodiment 26. The method of any one of embodiments 10-20, wherein the RGEN is pre-complexed with the gRNA, forming a Ribonucleoprotein (RNP) complex.

Embodiment 27. The method of embodiment 1, comprising delivering into the proliferative cell a nucleic acid construct comprising a donor nucleic acid, wherein the nucleic acid construct is configured such that insertion of the donor nucleic acid into the genome of the proliferative cell by homologous recombination decreases the helicase activity of WRN in the proliferative cell.

Embodiment 28. The method of embodiment 27, wherein the nucleic acid construct is an AAV vector.

Embodiment 29. The method of embodiment 28, wherein the AAV vector comprises two homology arms having sequences identical or substantially homologous to regions of the endogenous WRN gene.

Embodiment 30. The method of embodiment 28 or 29, wherein the AAV vector is an AAV clade F vector.

Embodiment 31. The method of embodiment 1, comprising delivering into the proliferative cell a proteolysis targeting chimera (PROTAC) that targets WRN for ubiquitination and proteolytic degradation.

Embodiment 32. The method of embodiment 31, wherein the PROTAC comprises an E3 ubiquitin ligase ligand coupled via a linker to a WRN ligand.

Embodiment 33. The method of any one of embodiments 1-32 wherein the proliferative cell comprises one or more MSI markers selected from the group consisting of BAT25, BAT26, D2S123, D5S346, and D17S250.

Embodiment 34. The method of any one of embodiments 1-33, wherein the proliferative cell comprises a mutation that impairs DNA mismatch repair.

Embodiment 35. The method of embodiment 34, wherein the proliferative cell comprises a mutation in a MutS homolog and/or a mutation in a MutL homolog.

Embodiment 36. The method of embodiment 35, wherein the MutS homolog is selected from the group consisting of MSH2, MSH3, and MSH6, and the MutL homolog is selected from the group consisting of MLH1, MLH3, PMS1, and PMS2.

Embodiment 37. The method of embodiment 36, wherein the proliferative cell comprises a mutation in MLH1, MSH2, and/or PMS2.

Embodiment 38. The method of any one of embodiments 1-37, wherein the proliferative cell comprises one or more markers of DNA damage.

Embodiment 39. The method of embodiment 38, wherein the one or more markers of DNA damage are selected from the group consisting of high p21 expression and high γH2AX expression.

Embodiment 40. The method of any one of embodiments 1-39, wherein decreasing proliferation in the proliferative cell comprises inducing cell cycle arrest in the proliferative cell.

Embodiment 41. The method of any one of embodiments 1-39, wherein decreasing proliferation in the proliferative cell comprises inducing apoptosis in the proliferative cell.

Embodiment 42. The method of any one of embodiments 1-41, wherein the method is carried out in vivo.

Embodiment 43. The method of any one of embodiments 1-41, wherein the method is carried out ex vivo.

Embodiment 44. The method of any one of embodiments 1-41, wherein the method is carried out in vitro.

Embodiment 45. The method of any one of embodiments 1-44, wherein the proliferative cell is a cancer cell.

Embodiment 46. The method of embodiment 45, wherein the cancer is selected from the group consisting of colon cancer, gastric cancer, endometrium cancer, ovarian cancer, hepatobiliary tract cancer, urinary tract cancer, brain cancer, and skin cancer.

Embodiment 47. The method of any one of embodiments 1-46, wherein the proliferative cell is (a) a mammalian cell; (b) a human cell; or (c) a veterinary animal cell.

Embodiment 48. A method for treating a proliferative disease in an individual in need thereof, the proliferative disease being characterized by proliferating cells having an MSI, comprising administering to the individual a pharmaceutical agent effective for decreasing the helicase activity of WRN in the proliferative cells.

Embodiment 49. The method of embodiment 48, comprising administering to the individual an inhibitor of WRN.

Embodiment 50. The method of embodiment 49, wherein the inhibitor of WRN is a small molecule inhibitor.

Embodiment 51. The method of embodiment 50, wherein the small molecule inhibitor has the formula:

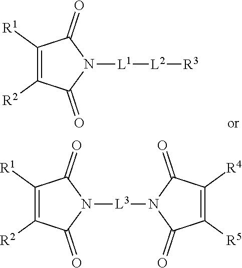

or a pharmaceutically acceptable salt thereof,
wherein
L¹ is C₁₋₄ alkylene;
L² is O, S, OC(O), OSO₂, OC(O)O, or OC(O)NH;
L³ is C₁₋₈ alkylene;
R¹, R², R⁴, and R⁵ are each independently H, halogen, C₁₋₄ alkyl, or C₁₋₄ haloalkyl; and R³ is H, C₁₋₆ alkyl, C₂₋₆ alkenyl, C₂₋₆ alkynyl, C₁₋₆ hydroxyalkyl, C₁₋₆ haloalkyl, C₆₋₁₂ aryl, or C₆₋₁₂ aryl-C₁₋₄ alkyl, each of which is optionally substituted with halogen, C₁₋₄ alkyl, or C₁₋₄ haloalkyl.

Embodiment 52. The method of embodiment 49, wherein the inhibitor of WRN is an ADC comprising an antibody conjugated to a WRN inhibitor.

Embodiment 53. The method of embodiment 48, comprising administering to the individual an inhibitory nucleic acid targeting WRN mRNA, or a nucleic acid encoding the inhibitory nucleic acid.

Embodiment 54. The method of embodiment 53, wherein the inhibitory nucleic acid comprises a short interfering RNA (siRNA), a microRNA (miRNA), or an antisense oligonucleotide.

Embodiment 55. The method of embodiment 48, comprising administering to the individual a nuclease capable of modifying the genomes of the proliferative cells such that the helicase activity of WRN in the proliferative cells is decreased, or a nucleic acid encoding the nuclease.

Embodiment 56. The method of embodiment 55, wherein the nuclease is a transcription activator-like effector nuclease (TALEN) or zinc-finger nucleases (ZFN) targeting a genomic sequence within or near an endogenous WRN gene locus.

Embodiment 57. The method of embodiment 55, comprising administering to the individual a) a gRNA comprising a spacer sequence that is complementary to a genomic sequence within or near an endogenous WRN gene locus, or a nucleic acid encoding the gRNA; and b) an RNA-guided endonuclease (RGEN), or a nucleic acid encoding the RGEN.

Embodiment 58. The method of embodiment 57, wherein the nucleic acid encoding the gRNA is contained in an Adeno Associated Virus (AAV) vector and/or the nucleic acid encoding the RGEN is contained in an AAV vector.

Embodiment 59. The method of embodiment 57 or 58, wherein the spacer sequence is complementary to a genomic sequence within a coding region of the endogenous WRN gene.

Embodiment 60. The method of any one of embodiments 57-59, wherein the genomes of the proliferative cells are modified by non-homologous end joining (NHEJ).

Embodiment 61. The method of any one of embodiments 57-59, further comprising administering to the individual a donor template comprising a donor nucleic acid, wherein the donor template is configured such that the donor nucleic acid is capable of being inserted into the WRN gene locus by homologous recombination.

Embodiment 62. The method of embodiment 61, wherein the donor nucleic acid encodes one or more STOP codons, and the donor template is configured such that the donor nucleic acid is inserted into a coding region of the WRN gene.

Embodiment 63. The method of embodiment 62, wherein the donor nucleic acid encodes three STOP codons in each of the 3 translation frames present in succession.

Embodiment 64. The method of embodiment 61, wherein the donor nucleic acid encodes a mutation in the WRN helicase domain that decreases WRN helicase activity.

Embodiment 65. The method of any one of embodiments 61-64, wherein the donor template is contained in an AAV vector.

Embodiment 66. The method of any one of embodiments 57-65, wherein the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof.

Embodiment 67. The method of embodiment 66, wherein the RGEN is Cas9.

Embodiment 68. The method of any one of embodiments 57-67, wherein the nucleic acid encoding the RGEN is a ribonucleic acid (RNA) sequence.

Embodiment 69. The method of embodiment 68, wherein the RNA sequence encoding the RGEN is linked to the gRNA via a covalent bond.

Embodiment 70. The method of any one of embodiments 57-67, wherein the nucleic acid encoding the RGEN is a deoxyribonucleic acid (DNA) sequence.

Embodiment 71. The method of any one of embodiments 57-70, wherein the nucleic acid encoding the RGEN is formulated in a liposome or lipid nanoparticle.

Embodiment 72. The method of embodiment 71, wherein the liposome or lipid nanoparticle encapsulates the gRNA.

Embodiment 73. The method of any one of embodiments 57-67, wherein the RGEN is pre-complexed with the gRNA, forming a Ribonucleoprotein (RNP) complex.

Embodiment 74. The method of embodiment 48, comprising administering to the individual a nucleic acid construct comprising a donor nucleic acid, wherein the nucleic acid construct is configured such that insertion of the donor nucleic acid into the genomes of the proliferative cells by homologous recombination decreases the helicase activity of WRN in the proliferative cells.

Embodiment 75. The method of embodiment 74, wherein the nucleic acid construct is an AAV vector.

Embodiment 76. The method of embodiment 75, wherein the AAV vector comprises two homology arms having sequences identical or substantially homologous to regions of the endogenous WRN gene.

Embodiment 77. The method of embodiment 75 or 76, wherein the AAV vector is an AAV clade F vector.

Embodiment 78. The method of embodiment 48, comprising administering to the individual a PROTAC that targets WRN for ubiquitination and proteolytic degradation.

Embodiment 79. The method of embodiment 78, wherein the PROTAC comprises an E3 ubiquitin ligase ligand coupled via a linker to a WRN ligand.

Embodiment 80. The method of any one of embodiments 48-79, wherein the proliferative cells comprise one or more MSI markers selected from the group consisting of BAT25, BAT26, D2S123, D5S346, and D17S250.

Embodiment 81. The method of embodiment 80, further comprising determining the presence of the one or more MSI markers in a population of proliferative cells from the individual to identify the presence of MSI in the proliferative cells.

Embodiment 82. The method of embodiment 81, wherein the step of determining the presence of the one or more MSI markers is carried out prior to administering the pharmaceutical agent.

Embodiment 83. The method of any one of embodiments 48-82, wherein the proliferative cells comprises a mutation that impairs DNA mismatch repair.

Embodiment 84. The method of embodiment 83, wherein the proliferative cell comprises a mutation in a MutS homolog and/or a mutation in a MutL homolog.

Embodiment 85. The method of embodiment 84, wherein the MutS homolog is selected from the group consisting of MSH2, MSH3, and MSH6, and the MutL homolog is selected from the group consisting of MLH1, MLH3, PMS1, and PMS2.

Embodiment 86. The method of embodiment 85, wherein the proliferative cell comprises a mutation in MLH1, MSH2, and/or PMS2.

Embodiment 87. The method of any one of embodiments 83-86, further comprising determining the presence of the mutation in a population of proliferative cells from the individual to identify the presence of the mutation in the proliferative cells.

Embodiment 88. The method of embodiment 87, wherein the step of determining the presence of the mutation is carried out prior to administering the pharmaceutical agent.

Embodiment 89. The method of any one of embodiments 48-88, wherein the proliferative cell comprises one or more markers of DNA damage.

Embodiment 90. The method of embodiment 89, wherein the one or more markers of DNA damage are selected from the group consisting of high p21 expression and high γH2AX expression.

Embodiment 91. The method of embodiment 89 or 90, further comprising determining the presence of the one or more markers of DNA damage in a population of proliferative cells from the individual to identify the presence of the one or more markers of DNA damage in the proliferative cells.

Embodiment 92. The method of embodiment 91, wherein the step of determining the presence of the one or more markers of DNA damage is carried out prior to administering the pharmaceutical agent.

Embodiment 93. The method of any one of embodiments 48-92, wherein the amount of proliferative cells in the individual is decreased as compared to a corresponding individual that does not receive administration of the pharmaceutical agent.

Embodiment 94. The method of any one of embodiments 48-93, wherein the rate of proliferation of the proliferative cells is decreased as compared to a corresponding individual that does not receive administration of the pharmaceutical agent.

Embodiment 95. The method of any one of embodiments 48-94, wherein at least some of the proliferative cells are induced to undergo cell cycle arrest.

Embodiment 96. The method of any one of embodiments 48-95, wherein at least some of the proliferative cells are induced to undergo apoptosis.

Embodiment 97. The method of any one of embodiments 48-96, wherein the proliferative disease is a cancer.

Embodiment 98. The method of embodiment 97, wherein the cancer is selected from the group consisting of colon cancer, gastric cancer, endometrium cancer, ovarian cancer, hepatobiliary tract cancer, urinary tract cancer, brain cancer, and skin cancer.

Embodiment 99. The method of any one of embodiments 48-98, further comprising administering to the individual a conventional therapy for the proliferative disease.

Embodiment 100. The method of embodiment 99, comprising administering to the individual an anti-PD-1 therapy.

Embodiment 101. The method of any one of embodiments 48-100, wherein the individual is (a) a mammal; (b) a human; or (c) a veterinary animal.

Embodiment 102. A method for predicting if an individual diagnosed with a proliferative disease is likely to respond to a therapy comprising administering to the individual a pharmaceutical agent effective for decreasing WRN helicase activity, the method comprising determining the presence of an MSI, or a marker associated with an MSI, in a population of proliferative cells from the individual, and determining a likelihood that the individual will respond to the therapy based on the determination of the presence of MSI, or a marker associated with MSI, in the population of proliferative cells.

Embodiment 103. The method of embodiment 102, wherein the determination of the presence of MSI in the population of proliferative cells comprises determining the presence of one or more MSI markers selected from the group consisting of BAT25, BAT26, D2S123, D5S346, and D17S250.

Embodiment 104. The method of embodiment 102 or 103, wherein the individual is predicted to respond to the therapy if the amount of cells in the population of proliferative cells determined to have at least one of the MSI markers is above a pre-determined threshold for the proliferative disease.

Embodiment 105. The method of embodiment 102 or 103, wherein the individual is predicted not to respond to the therapy if the amount of cells in the population of proliferative cells determined to have at least one of the MSI markers is below a pre-determined threshold for the proliferative disease; or the population of proliferative cells is determined to have none of the MSI markers.

Embodiment 106. The method of embodiment 102, wherein the determination of the presence of a marker associated with MSI in the population of proliferative cells comprises determining the presence of a mutation that impairs DNA mismatch repair.

Embodiment 107. The method of embodiment 106, wherein the mutation comprises a mutation in a MutS homolog and/or a mutation in a MutL homolog.

Embodiment 108. The method of embodiment 107, wherein the MutS homolog is selected from the group consisting of MSH2, MSH3, and MSH6, and the MutL homolog is selected from the group consisting of MLH1, MLH3, PMS1, and PMS2.

Embodiment 109. The method of embodiment 108, wherein the mutation comprises a mutation in MLH1, MSH2, and/or PMS2.

Embodiment 110. The method of embodiment 102, wherein the determination of the presence of a marker associated with MSI in the population of proliferative cells comprises determining the presence of one or more markers of DNA damage.

Embodiment 111. The method of embodiment 110, wherein the one or more markers of DNA damage are selected from the group consisting of high p21 expression and high γH2AX expression.

Embodiment 112. The method of any one of embodiments 106-111, wherein the individual is predicted to respond to the therapy if the amount of cells in the population of proliferative cells determined to have (i) at least one mutation that impairs DNA mismatch repair and/or (ii) at least one marker of DNA damage is above a pre-determined threshold for the proliferative disease.

Embodiment 113. The method of embodiment 112, wherein the at least one mutation that impairs DNA mismatch repair comprises a mutation in MLH1, MSH2, and/or PMS2, and the at least one marker of DNA damage comprises high p21 expression and/or high γH2AX expression.

Embodiment 114. The method of embodiment 106-111, wherein the individual is predicted not to respond to the therapy if the amount of cells in the population of proliferative cells determined to have (i) at least one mutation that impairs DNA mismatch repair and/or (ii) at least one marker of DNA damage is below a pre-determined threshold for the proliferative disease; or the population of proliferative cells is determined to have no mutations that impair DNA mismatch repair and no DNA damage markers.

Embodiment 115. A method for detecting a microsatellite instability (MSI) and the helicase activity of WRN in an individual diagnosed with or thought to have a proliferative disease, the method comprising: (a) contacting a biological sample from the individual with one or more reagents for detecting the presence of an MSI and the helicase activity of WRN; and (b) detecting (i) the presence of an MSI; and (ii) the helicase activity of WRN.

Embodiment 116. The method of embodiment 115, wherein the reagent for detecting the presence of an MSI in a biological sample comprises a reagent for detecting the presence of one or more MSI markers selected from the group consisting of BAT25, BAT26, D2S123, D5S346, and D17S250.

Embodiment 117. A method for detecting a marker associated with an MSI and the helicase activity of WRN in an individual diagnosed with or thought to have a proliferative disease, the method comprising: (a) contacting a biological sample from the individual with one or more reagents for detecting the presence of a marker associated with an MSI and the helicase activity of WRN helicase; and (b) detecting (i) the presence of the marker associated with an MSI; and (ii) the helicase activity of WRN helicase.

Embodiment 118. The method of embodiment 117, wherein the reagent for detecting the presence of a marker associated with an MSI in a biological sample comprises a reagent for detecting the presence of (i) one or more mutations that impair DNA mismatch repair and/or (ii) one or more markers of DNA damage.

Embodiment 119. The method of embodiment 118, wherein the one or more mutations that impair DNA mismatch repair comprise a mutation in a MutS homolog and/or a mutation in a MutL homolog.

Embodiment 120. The method of embodiment 119, wherein the MutS homolog is selected from the group consisting of MSH2, MSH3, and MSH6, and the MutL homolog is selected from the group consisting of MLH1, MLH3, PMS1, and PMS2.

Embodiment 121. The method of embodiment 120, wherein the one or more mutations comprise a mutation in MLH1, MSH2, and/or PMS2.

Embodiment 122. The method of any one of embodiments 118-121, wherein the one or more markers of DNA damage are selected from the group consisting of high p21 expression and high γH2AX expression.

Embodiment 123. The method of any one of embodiments 102-122, wherein the proliferative disease is a cancer.

Embodiment 124. The method of embodiment 123, wherein the cancer is selected from the group consisting of colon cancer, gastric cancer, endometrium cancer, ovarian cancer, hepatobiliary tract cancer, urinary tract cancer, brain cancer, and skin cancer.

Embodiment 125. The method of any one of embodiments 102-124, wherein the individual is (a) a mammal; (b) a human; or (c) a veterinary animal.

Embodiment 126. A composition comprising (a) a gRNA comprising a spacer sequence complementary to a genomic sequence within or near an endogenous WRN gene locus, or a nucleic acid encoding the gRNA; and b) an RNA-guided endonuclease (RGEN), or a nucleic acid encoding the RGEN, wherein the components of the composition are configured such that delivery of the composition into a cell is capable of decreasing the helicase activity of WRN in the cell.

Embodiment 127. The composition of embodiment 126, wherein the nucleic acid encoding the gRNA is contained in an Adeno Associated Virus (AAV) vector and/or the nucleic acid encoding the RGEN is contained in an AAV vector.

Embodiment 128. The composition of embodiment 126 or 127, wherein the spacer sequence is complementary to a genomic sequence within a coding region of the endogenous WRN gene.

Embodiment 129. The composition of any one of embodiments 126-128, further comprising a donor template comprising a donor nucleic acid, wherein the donor template is configured such that the donor nucleic acid is capable of being inserted into the WRN gene locus by homologous recombination.

Embodiment 130. The composition of embodiment 129, wherein the donor nucleic acid encodes one or more STOP codons, and the donor template is configured such that the donor nucleic acid is inserted into a coding region of the WRN gene.

Embodiment 131. The composition of embodiment 130, wherein the donor nucleic acid encodes three STOP codons in each of the 3 translation frames present in succession.

Embodiment 132. The composition of embodiment 129, wherein the donor nucleic acid encodes a mutation in the WRN helicase domain that decreases WRN helicase activity.

Embodiment 133. The composition of any one of embodiments 129-132, wherein the donor template is contained in an AAV vector.

Embodiment 134. The composition of any one of embodiments 126-133, wherein the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof.

Embodiment 135. The composition of embodiment 134, wherein the RGEN is Cas9.

Embodiment 136. The composition of any one of embodiments 126-135, wherein the nucleic acid encoding the RGEN is a ribonucleic acid (RNA) sequence.

Embodiment 137. The composition of embodiment 136, wherein the RNA sequence encoding the RGEN is linked to the gRNA via a covalent bond.

Embodiment 138. The composition of any one of embodiments 126-135, wherein the nucleic acid encoding the RGEN is a deoxyribonucleic acid (DNA) sequence.

Embodiment 139. The composition of any one of embodiments 126-136, wherein the nucleic acid encoding the RGEN is formulated in a liposome or lipid nanoparticle.

Embodiment 140. The composition of embodiment 139, wherein the liposome or lipid nanoparticle encapsulates the gRNA.

Embodiment 141. The composition of any one of embodiments 126-135, wherein the RGEN is pre-complexed with the gRNA, forming a Ribonucleoprotein (RNP) complex.

Embodiment 142. A composition comprising a nucleic acid construct comprising a donor nucleic acid, wherein the nucleic acid construct is configured such that insertion of the donor nucleic acid into the genome of a proliferative cell by homologous recombination decreases the helicase activity of WRN in the proliferative cell.

Embodiment 143. The composition of embodiment 142, wherein the nucleic acid construct is an AAV vector.

Embodiment 144. The composition of embodiment 143, wherein the AAV vector comprises two homology arms having sequences identical or substantially homologous to regions of the endogenous WRN gene.

Embodiment 145. The composition of any one of embodiments 142-144, wherein the AAV vector is an AAV clade F vector.

The present disclosure has been described above with reference to specific alternatives. However, other alternatives than the above described are equally possible within the scope of the disclosure. Different method steps than those described above, may be provided within the scope of the disclosure. The different features and steps described herein may be combined in other combinations than those described.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

In addition, where features or aspects of the disclosure are described in terms of Markush groups, those skilled in the art will recognize that the disclosure is also thereby described in terms of any individual member or subgroup of members of the Markush group.

Any of the features of an alternative of an aspect is applicable to all aspects and alternatives identified herein. Moreover, any of the features of an alternative of an aspect is independently combinable, partly or wholly with other alternatives described herein in any way, e.g., one, two, or three or more alternatives may be combinable in whole or in part. Further, any of the features of an alternative of an aspect may be made optional to other aspects or alternatives. Although described above in terms of various example alternatives and implementations, it should be understood that the various features, aspects and functionality described in one or more of the individual alternatives are not limited in their applicability to the particular alternative with which they are described, but instead may be applied, alone or in various combinations, to one or more of the other alternatives of the present application, whether or not such alternatives are described and whether or not such features are presented as being a part of a described alternative. Thus, the breadth and scope of the present application should not be limited by any of the above-described example alternatives.

All references cited herein are incorporated herein by reference in their entirety. To the extent publications and patents or patent applications incorporated by reference contradict the disclosure contained in the specification, the specification is intended to supersede and/or take precedence over any such contradictory material. To the extent publications and patents or patent applications incorporated by reference herein contradict the disclosure contained in the specification, the specification is intended to supersede and/or take precedence over any such contradictory material.

The details of one or more embodiments of the disclosure are set forth in the accompanying description below. Any materials and methods similar or equivalent to those described herein can be used in the practice or testing of the present disclosure. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Some embodiments of the disclosures provided herewith are further illustrated by the following non-limiting examples.

### EXAMPLES

### Example 1: WRN Synthetic Lethal (SL) Interactions

This example probes SL interactions of the RECQ helicase, WRN, and compares such SL interactions to those of another RECQ helicase, BLM.

### Materials and Methods

Potential SL interactions were tested between BLM and WRN, and clinically relevant DDR proteins such as PARP, MLH1, FBXW7 and ATM, etc. (Table 1).

TABLE 1
Candidate approach to identifying BLM and WRN synthetic lethal interactions

| Gene | Testing tools |
|---|---|
| PARP | Olaparib on HAP1 BLM/WRN isogenic cells |
| | Olaparib and talazoparib on BLM/WRN siRNA transfected Hs578T cells |
| ATM | KU55933 and KU60019 on HAP1 BLM/WRN isogenic cells |
| | BLM/WRN siRNA transfected ATM null cell lines (Hs695T, SKCO-1) |
| | Dual siRNA in A549 cells |
| DNAPK | NU7447 on HAP1 BLM/WRN isogenic cells |
| | BLM/WRN siRNA transfected HAP1 DNAPK isogenic cells |
| Wee1 | MK-1775 on HAP1 BLM/WRN isogenic cells |
| CHEK1 | MK-8776 on HAP1 BLM/WRN isogenic cells |
| CHEK1&2 | AZD7762 on HAP1 BLM/WRN isogenic cells |
| FANCD2 | siRNA transfected HAP1 BLM/WRN isogenic cells |
| FBXW7 | siRNA transfected HAP1 BLM/WRN isogenic cells |
| | Dual siRNA in A549 cells |
| XRCC3 | siRNA transfected HAP1 BLM/WRN isogenic cells |
| RAD54B | siRNA transfected HAP1 BLM/WRN isogenic cells |
| NBN | siRNA transfected HAP1 BLM/WRN isogenic cells |
| LIG4 | siRNA transfected HAP1 BLM/WRN isogenic cells |
| ERCC5 | siRNA transfected HAP1 BLM/WRN isogenic cells |
| TP53BP1 | siRNA transfected HAP1 BLM/WRN isogenic cells |
| FANCM | siRNA transfected HAP1 BLM/WRN isogenic cells |
| MLH1 | BLM/WRN siRNA transfected HAP1 MLH1 isogenic cells |
| | Dual siRNA in A549 cells |
| MSH2 | Dual siRNA in A549 cells |
| PMS1 | Dual siRNA in A549 cells |
| PMS2 | Dual siRNA in A549 cells |

Three approaches were used in proliferation assays. The first, was treating isogenic HAP1 parental and BLM or WRN CRISPR knockout (KO) cell lines with commercially available tool compounds. The second, was transfecting HAP1 parental and WRN/BLM KO cells with siRNAs of potential SL partners or transfecting WRN and BLM siRNAs into KO cells of potential SL partners. Lastly, dual siRNA experiments were carried out which involves cotransfecting siRNAs individually and together.

Cell lines: HAP 1 isogenic cell lines were obtained from Horizon Discovery (BLM; HZGHC000629c007, WRN; HZGHC000432c001). All other lines were obtained from ATCC. Stable cell lines were generated using lentiviral infection by cloning into a pLVbsd-EF1 a-HA vector. Cloning and lentiviral particle generation was carried out at Biosiettia Inc. Cells were seeded into a 6-well plate (225,000 cells/well). The next day, cells were infected with virus and polybrene (8 μg/ml) at a MOI of 5 for WRN rescue experiments and MOI of 10 for MLH1 and MRE11 rescue experiments. 48 h later, cells were seeded into media containing 10 μg/ml of blasticidin to select for infected cells.

Transfections: siRNAs were obtained from Life technologies (Table 2).

TABLE 2
siRNAs

| Gene | siRNA ID# | Antisense sequence |
|---|---|---|
| WRN | s14907 | AGUAAGAUAGAAACCCUCCgt |
| WRN 5′UTR | NM_000553_ | AAACCCGAGAAGAUCCAGUCC |
| | stealth_691 | AACA |
| BLM | s1999 | UUUCGUUUUGGAAGAUAUCtt |
| MLH 1.1 | s297 | AUAUUGUCCACGGUUGAGGca |
| MLH 1.2 | s298 | UAUUGUCCACGGUUGAGGCat |
| MLH 1.3 | s224048 | UAUCCUCACAUCCAAUUUCta |
| MSH2 1.1 | s8966 | UUACACGAAAGUAAUAUCCaa |
| MSH2 1.2 | s8967 | UAAGAUCUGGGAAUCGACGaa |
| MSH2 1.3 | s8968 | UAUCAUAUCCUUGCGAUUCtc |
| PMS1 1.1 | s229950 | UCUACAUUCAUAAACUUCCtt |
| PMS1 1.2 | s229951 | ACAAGUUUUACUAUAUUCCgt |
| PMS1 1.3 | s229952 | UCUUUUAAAUCUGCUACUCca |
| PMS2 1.1 | s10740 | AUUGGUGCAACUUACACGGat |
| PMS2 1.2 | s10742 | AAACUCGAAAUUUACAUCCgg |
| PMS2 1.3 | s534928 | UCUUGUAGCAAAAUUUGCCtt |
| Kif11 | s7903 | UGAACUUAGAAGAUCAGUCtt |

Cells were transfected with 5 nM of negative control siRNA #1 (catalog #4390842) or relevant siRNA. Kifl 1 is an essential gene in dividing cells. Cells were counted and seeded into a 24-well plate (50,000 cells/well). Approximately 8 h later, they were transfected with siRNA. After 3-4 days, the control siRNA transfected cells were counted and seeded into a 96-well plate (500-1000 cells/well) in triplicate. The remaining cells were used to determine degree of knock down using RTPCR. Gene-targeting siRNA transfected cells were seeded using the volume calculated for the control siRNA cells to maintain the effects that occurred during the 1st 4 days of knockdown. The next day, the cells were transfected again and monitored over 4-5 days in an IncuCyte ZOOM instrument.

RT-PCR: Taqman Probes were obtained from Life technologies (Table 3). RNA was isolated using an RNAeasy purification kit (Qiagen; 74106). 100 ng of RNA was used in a reverse transcription reaction (Life technologies; Ser. No. 11/756,500). The resulting cDNA was diluted 2 fold and added to taqman gene expression master mix (Life technologies; 4369016) containing the internal gene control probe against PPIA (VIC) and target gene probe (FAM) following the vendor's manual.

TABLE 3
Taqman Probes

| Gene | Probe ID |
|---|---|
| WRN | Hs00172155 |
| BLM | Hs00172060 |
| MLH1 | Hs00979919 |
| MSH2 | Hs00953527 |
| PMS1 | Hs00922262 |
| PMS2 | Hs00241053 |
| PPIA | Hs04194521 |

### Results

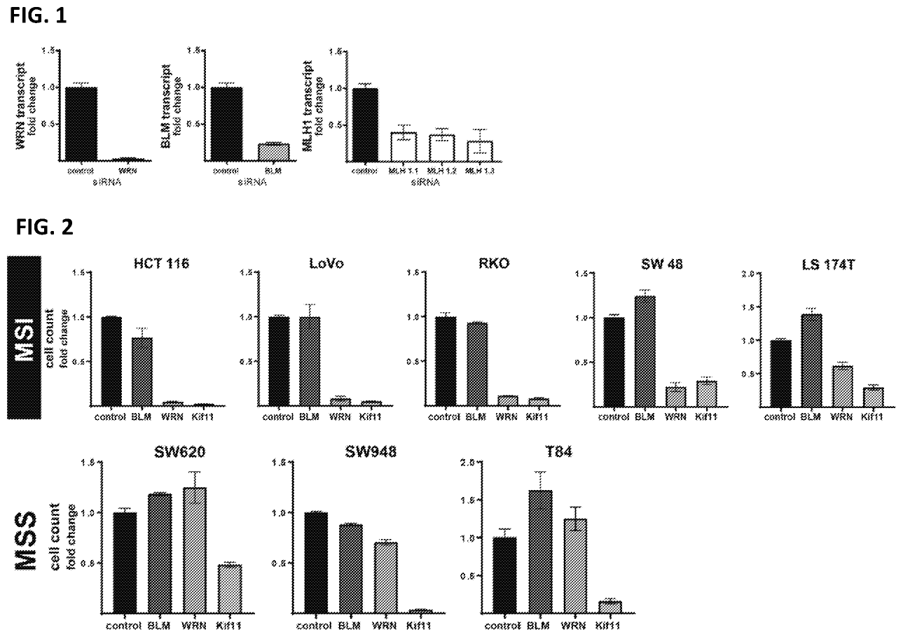

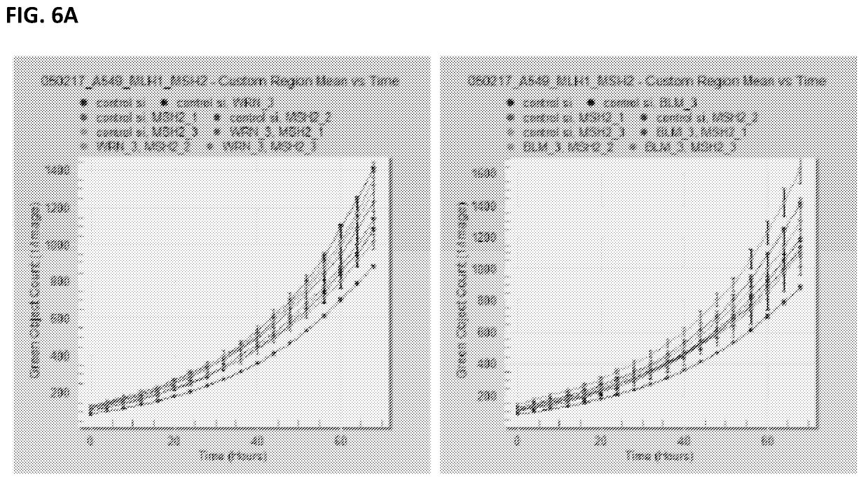

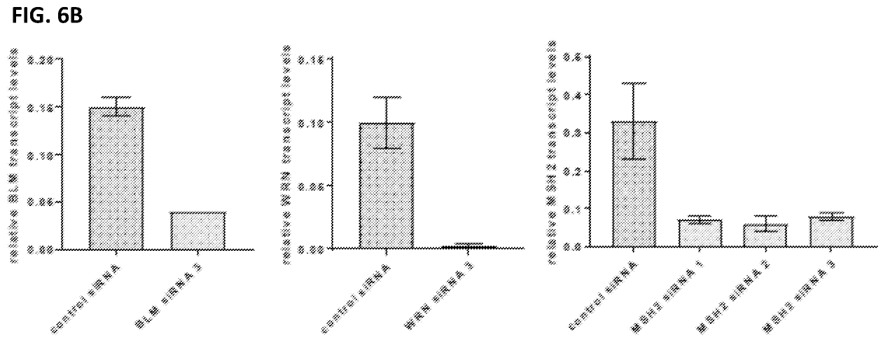

Three siRNAs targeting MLH1 showed efficient knockdown of MLH1 transcript as did the BLM and WRN siRNA on BLM and WRN transcript, respectively (FIG. 1). During MMR, MSH2 exists in a complex with MSH6 and MLH1 exists in a complex with PMS2 (Li G M. Cell Res 2008; 18(1):85-98). When MSH2 or MLH1 expression is decreased, MSH6 and PMS2 are destabilized suggesting that MSH2 and MLH1 are core components of the protein complexes that detect DNA mismatches. Accordingly, we evaluated whether dual knockdown of MSH2 and WRN would synergize to result in synthetic lethality. Dual siRNA with MSH2 and WRN did not result in synthetic lethality (FIG. 6A and FIG. 6B). Additionally, PMS1 and PMS2 did not result in loss of proliferation synergy (FIG. 6A and FIG. 6B).

### Example 2: MMR-Deficient Cell Lines are Sensitive to WRN Knockdown

The protein expression of MLH1 and other MMR proteins is lost in some colorectal cancer cell lines. This example tests if MMR-deficient/MSI cell lines were sensitive to WRN compared to MMR-proficient/microsatellite stable (MSS) cell lines.

### Materials and Methods

Proliferation and viability assays: Cell lines were infected with NucLight Red lentivirus (Essen Biosciences; 4476) to express a red fluorescent protein in the nucleus thus enabling live-cell counting. Cells were counted, transfected with siRNA and monitored in an IncuCyte ZOOM instrument.

Other experiments performed as described above.

### Results

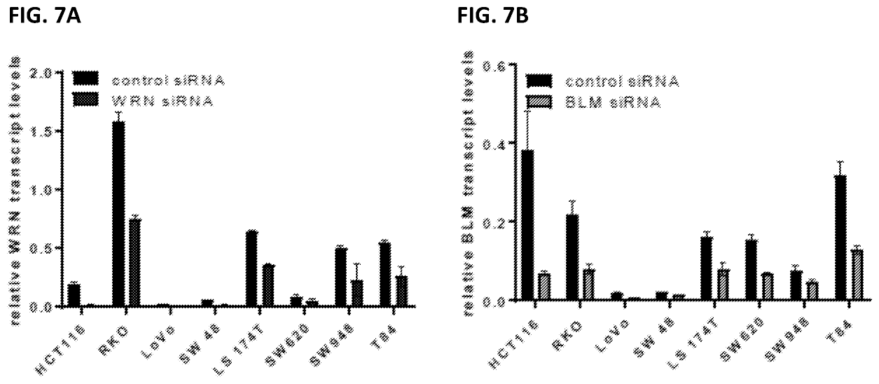

Five MMR-deficient/MSI cell lines, HCT116, LoVo, RKO, SW48, and LS174T, were found to be sensitive to WRN knockdown in 7- or 10-day viability assays (FIG. 2, top row). Conversely, MMR-proficient/MSS cell lines SW620, SW948, and T84 were found to be insensitive to WRN knockdown (FIG. 2, bottom row). Knockdown levels were confirmed using RT-PCR (FIG. 7A and FIG. 7B). In the dual siRNA experiments, MSH2 was not SL with WRN (FIG. 6A), however, a cell line with mutations in MSH2, LoVo, is sensitive to WRN knockdown (FIG. 2). The sensitivity of an MSH2 mutant cell line also suggests that WRN may be SL with downstream MSI.

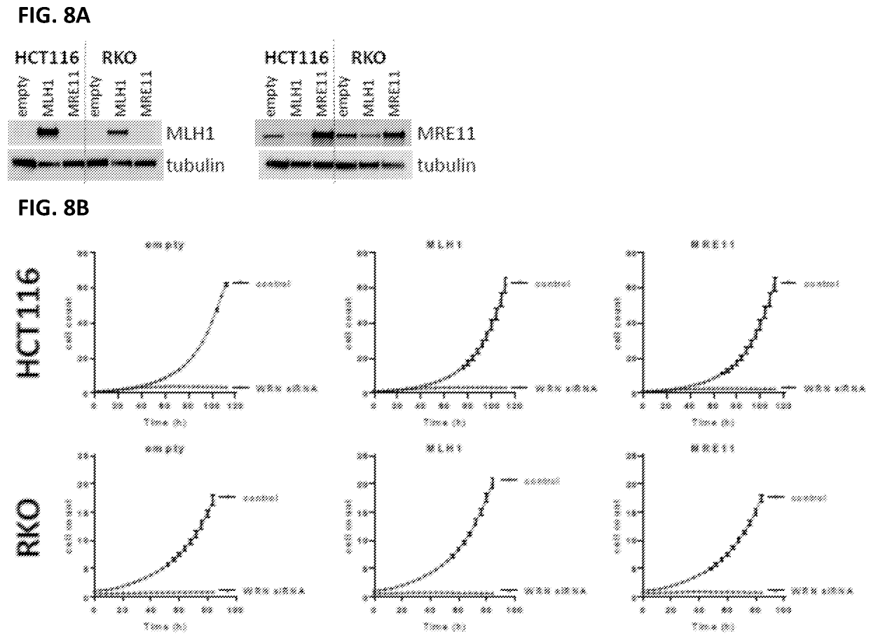

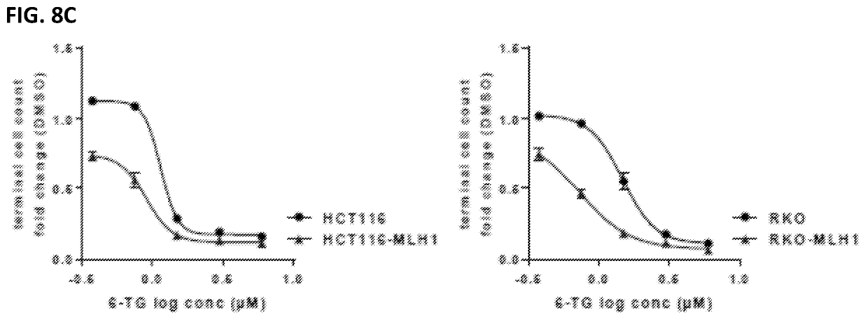

Since some MSI cell lines with homozygous mutations in MRE11 have previously been shown to be sensitive to WRN knockdown, we tested whether WRN was SL with MRE11. Knocking down MRE11 in DLD-1 cells, which do not express MSH6 and only have one WT copy of MRE11, did not render the cells sensitive to WRN loss (data not shown). Furthermore, reexpressing MRE11 in WRN-sensitive MSI cell lines (FIG. 8A) did not rescue the WRN siRNA phenotype (FIG. 8B). The WRN phenotype was unable to be rescued by reexpressing MLH1. Without being bound by theory, it is possible that reexpression was not sufficient to restore MMR and/or reverse the MSI. MMR-proficient cells are sensitive, while MMR-deficient cells are resistant, to 6-thioguanine (Yan T, Berry S E, Desai A B, Kinsella T J., Clin Cancer Res 2003; 9(6):2327-34). MLH1 reexpressing cells were found to be more sensitive to 6-TG (FIG. 8C) indicating that MMR was able to be rescued but not the WRN siRNA phenotype.

### Example 3: WRN Knockdown Increases DSB and Changes the Cell Cycle of MSI Cells

This Example tests how WRN knockdown affects double stranded DNA breaks and the cell cycle in MSI cell lines.

### Materials and Methods

Antibodies: WRN (Bethyl labs; A300-239A), Tubulin (LiCOR; 926-42211), γH2AX (Millipore; 05-636), p21 (abcam; ab109520), phospho-H3 488 (Cell signaling; 34655).

Immunofluorescence: Cell were seeded into 96-well plates (2000 cells/well), transfected 24 h, 48 h and 72 h prior to being fixed with 3.7% formaldehyde. 0.5% Triton-X was used to permeabilize the cell prior to incubation with primary antibodies overnight. Images were collected using a high-content INCELL imager.

Flow cytometry: Cell were seeded into 6-well plates (100,000-150,000 cells/well), transfected 48 h and 72 h. To detect cells in S-phase, we used a Click-iT EdU kit (Life technologies; C10425). Cell were incubated for 2 h with 10 μM EdU. To detect cells in M phase, transfected cells were fixed with 3.7% formaldehyde, permeabilized with 0.5% Triton-X and incubated with an anti-phospho H3 antibody. To measure DNA content, DRAQ7 (Abcam; ab109202) was added to the cells prior to analysis.

Other experiments performed as described above.

### Results

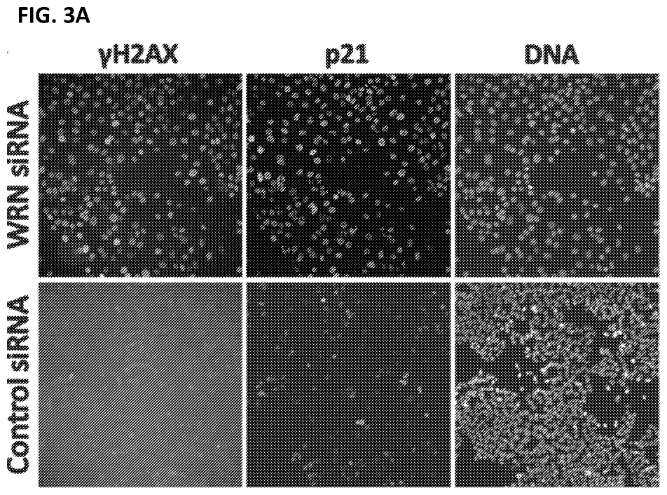

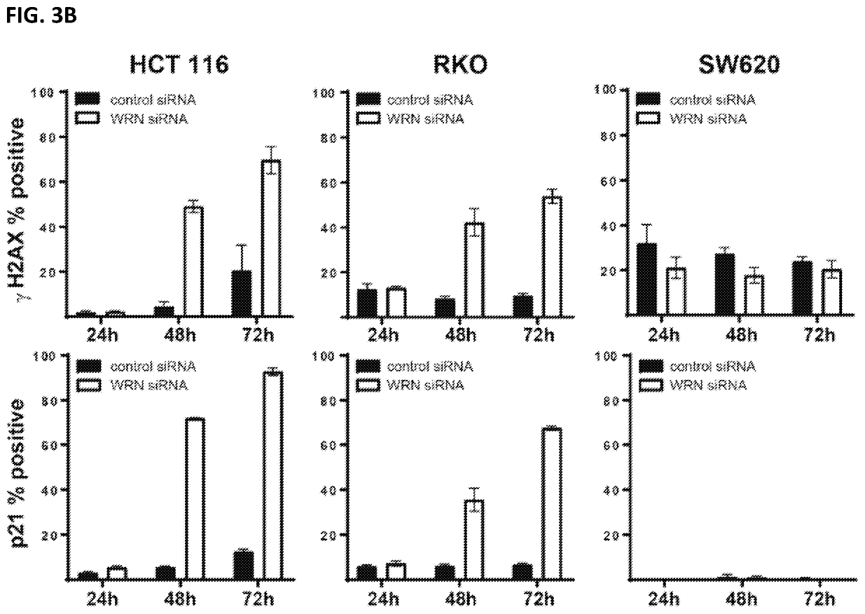

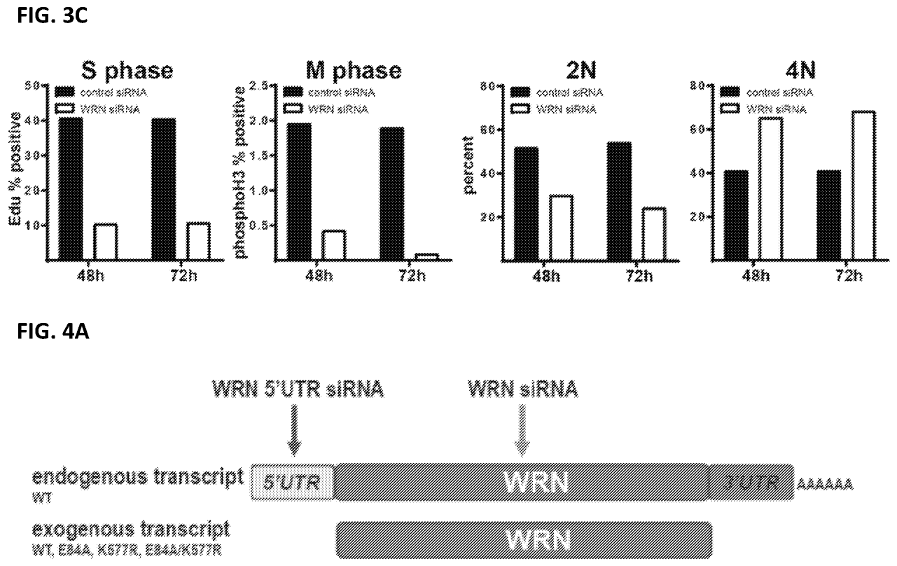

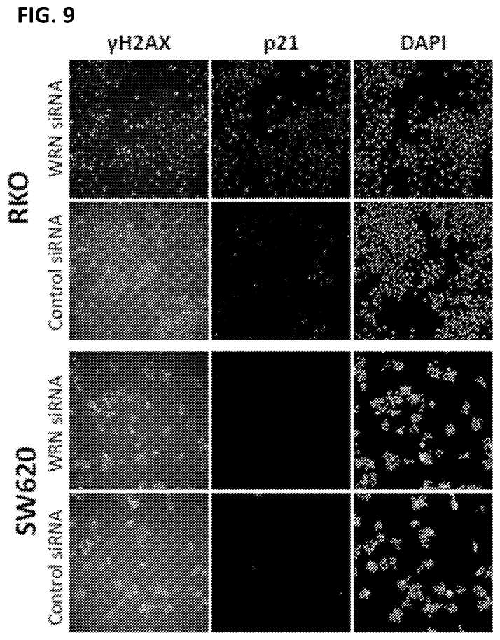

To understand the mechanism by which WRN knockdown was inducing loss of viability in MSI cells, transfected cells were harvested at 24, 48 and 72 h and stained using γH2AX and p21 antibodies. Compared to control siRNA, more HCT116 (FIG. 3A) and RKO (FIG. 9) cells transfected with WRN siRNA showed positive staining for both γH2AX and p21. SW620 (MSS) cells, on the other hand, did not show any change in γH2AX levels (FIG. 9). Quantification of antibody staining for all 3 cell lines is shown in FIG. 3B. p21 was not detected in SW620 cells that are have mutations in TP53. Since p21 is a p53 target gene, and without being bound by theory, this might explain the inability to detect p21. p21 regulates the cell cycle acting at the G1 to S checkpoint. Interestingly, most MSI cell lines except DLD-1 cells are WT for TP53 while MSS cell lines are mostly mutant for TP53 (Ahmed D, Eide P W, Eilertsen I A, Danielsen S A, Eknaes M, Hektoen M, et al., Oncogenesis 2013; 2:e71 doi 10.1038/oncsis.2013.35).

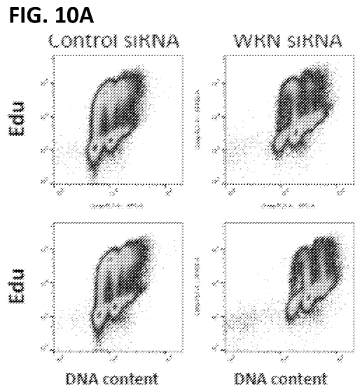

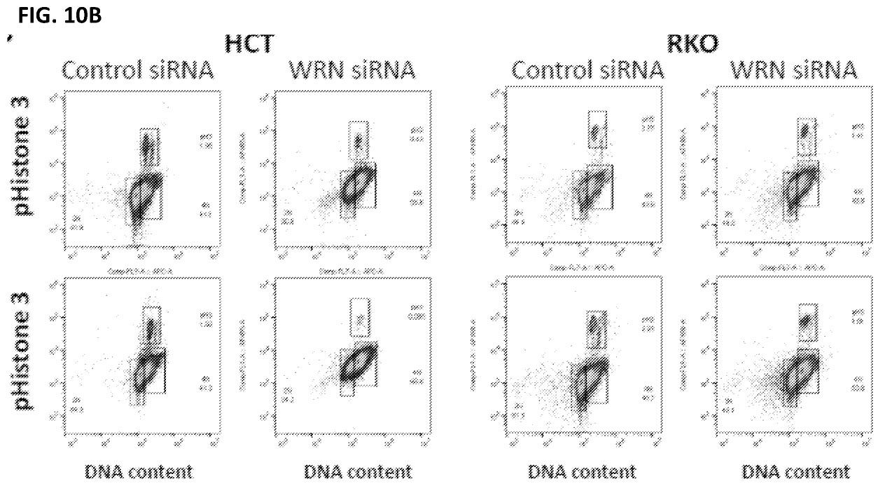

The robust increase in p21 levels following WRN loss in MSI cells prompted the measurement of cell cycle changes in these cells. Flow cytometry analyses revealed a decrease in cells entering S-phase consistent with elevated levels of p21. A decrease in cells in M-phase and with 2N DNA was also observed but, an increase in cells with 4N DNA (FIG. 3C, FIG. 10A, and FIG. 10B). The increase in 4N cells is consistent with the cells not going through mitosis. In summary, reducing WRN expression causes an increase in DSBs which in turn leads to changes in the cell cycle that slow proliferation.

### Example 4: WRN Helicase Domain can Rescue WRN Knockdown Phenotype in MSI Cells

This Example probes which domain is required for the WRN MSI SL interaction.

### Materials and Methods

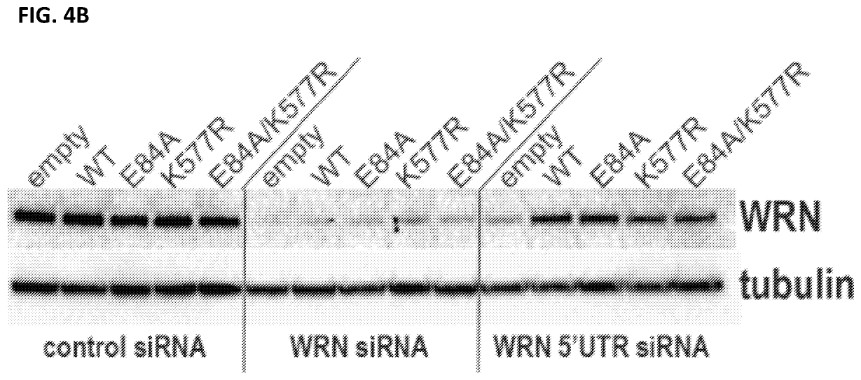

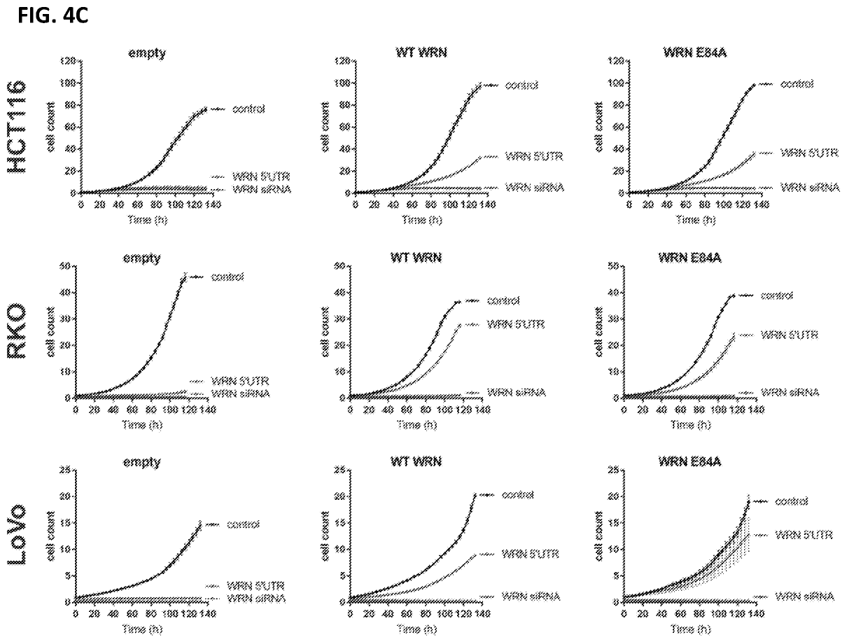

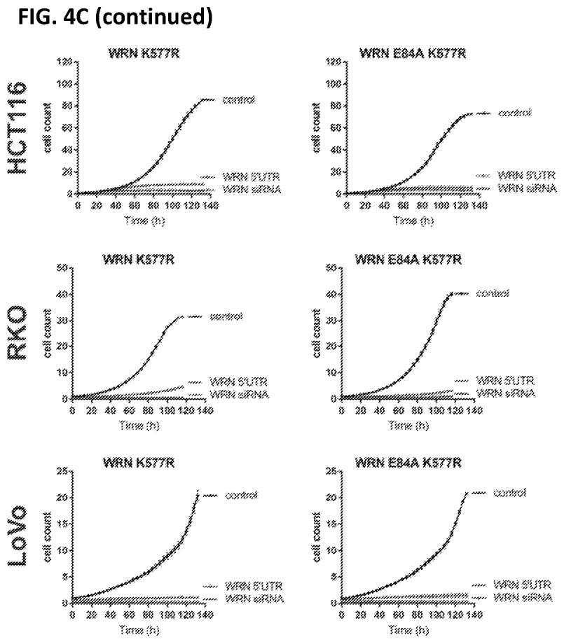

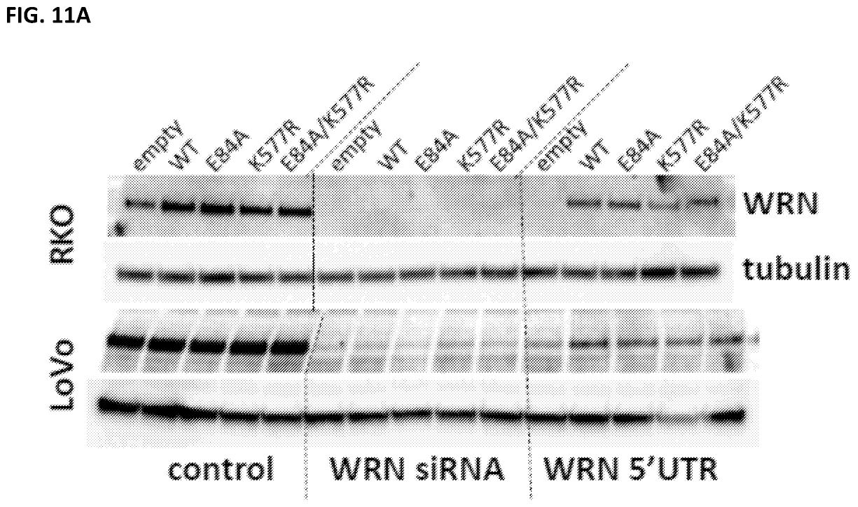

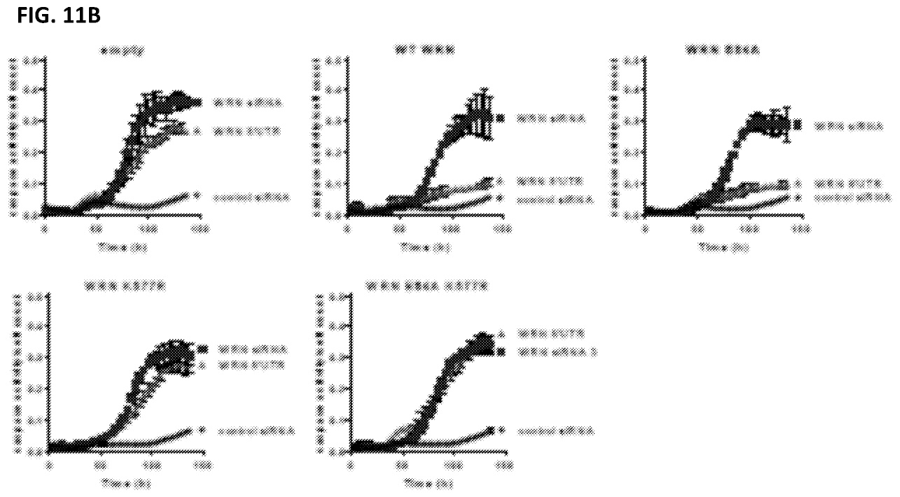

Cell lines constitutively expressing siRNA-resistant wild-type (WT), exonuclease-dead (E84A), helicase-dead (K577R) and enzymatically-dead (E84A/K577R) WRN were generated. The cells were then transfected with siRNA against the 5′UTR or in exon 8 of WRN mRNA (FIG. 4A). An siRNA-resistant pool of WRN was detected by western blot using the 5′UTR siRNA showing that the system is working as intended (FIG. 4B and FIG. 11B).

Other experiments performed as described above.

### Results

Knockdown with both siRNAs decreased the proliferation of the negative control cell lines. Partial to almost complete rescue with WT and exonuclease-dead WRN was observed in cells transfected with of the 5′UTR siRNA (FIG. 4C). Conversely, the helicase-dead and enzymatically-dead WRN did not rescue the loss of proliferation observed with the 5′UTR siRNA. It was also found that WRN knockdown increased the relative activity of caspases 3 and 7 in HCT 116 cells (FIG. 11A). Consistent with the rescue of proliferation, WT and exonuclease-dead WRN, and not helicase-dead mutants, rescued the increase in caspase activity in cells transfected with the 5′UTR siRNA. These results indicate that the helicase activity of WRN is driving the WRN SL interaction.

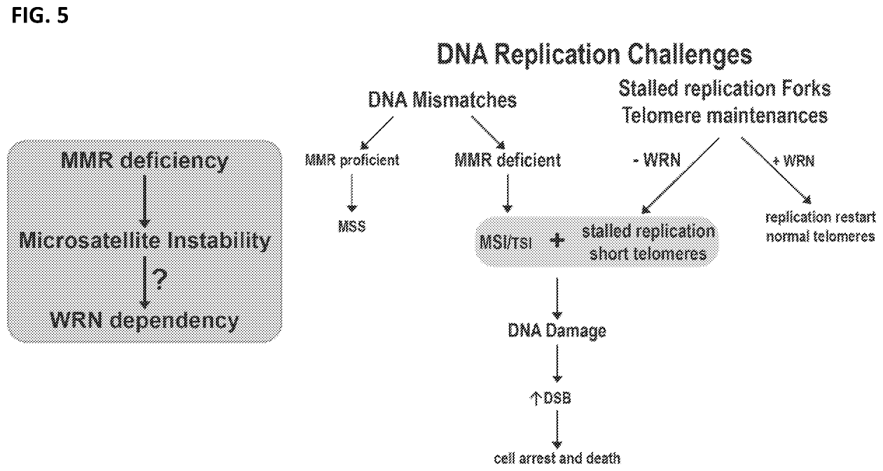

These results have shown that cells with MSI are dependent on WRN (FIG. 5, grey box). Without being bound by theory, the following model is proposed to explain the WRN MSI interaction. During DNA replication, MSI cells without WRN cannot restart replication and maintain their telomeres (FIG. 5). Aside from MSI, loss of MLH1 has been reported (Jia P, Chastain M, Zou Y, Her C, Chai W., Nucleic Acids Res 2017; 45(3):1219-32) to increase intrachromosomal telomere sequence insertions (TSI). The combination of MSI, TSI, stalled replication forks and shorter telomeres leads to unrepairable DNA damage indicated at least in part by more double stranded breaks. The accumulation of DSB triggers p21 cell arrest and apoptotic cell death.

### Example 5: Effects of NSC Compounds on MSI Vs MSS Cell Lines

This example tests the effects of known small molecule inhibitors of WRN on cell lines characterized as having MSI.

### Materials and Methods

Cell lines were infected with NucLight Red lentivirus (Essen Biosciences cat #4476) to express a red fluorescent protein in the nucleus thus enabling live-cell counting. Two cell lines with high microsatellite instability (MSI), HCT116 and RKO, and two microsatellite stable (MSS) cell lines, SW620 and SW948, were treated with WRN inhibitors, NSC 19630 (Calbiochem cat #681647) and NSC 617145 (Sigma cat #SML 1005) as follows. Cells were counted and seeded in triplicate into a 96-well plate (500 cells/well for MSI and 1000 cells/well for MSS). The next day, the cells were treated with compounds and monitored over 6 days in an IncuCyte ZOOM instrument. The cell count from the last time point was used to calculate IC₅₀ values using GraphPad Prism 7.

### Results

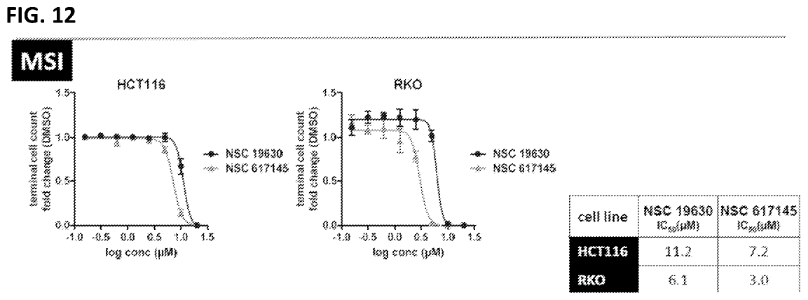

The effect of NSC 19630 and NSC 617145 on MSI cell lines is shown in FIG. 12. Consistent with the previously-described experiments with siRNA treatment (e.g., FIG. 2), exposure of MSI cell lines to the small molecules caused decreased proliferation of cells. The data from the MSS cell lines, SW620 and SW948, showed differences in sensitivity to the inhibitors, potentially suggesting non-MSI related or off-target effects at the higher concentrations in some cell lines.

### Example 6: Effects of WRN Knockdown in a Human Xenograft Mouse Model of Colon Carcinoma

This example tests the effects of knocking down the expression of WRN in xenograft tumors in mice derived from human colon carcinoma cell line HCT-116.

### Materials and Methods

Generation of WRN shRNA cell lines: HCT 116 cells were infected with lentivirus in the presence of polybrene (8 μg/ml) to express a control shRNA or 3 inducible WRN shRNAs at a MOI of 5. Lentiviral particles were purchased from Dharmacon (cat #V3 SH7669-225815112, V3 SH7669-225872565, V3 SH7669-226110693). Following antibiotic selection with puromycin (2 μg/ml), clones were selected using limited dilution. Ten clones per shRNA were characterized for WRN protein knockdown (westerns) and viability in the presence and absence of doxycycline (0.5 μg/ml). Two control shRNA and eight shRNAs that showed similar growth curves were further characterized for WRN transcript knockdown (RTPCR) and viability. One control and two WRN shRNAs were selected for implantation in vivo.

Female SCID mice are obtained (e.g., from Charles River Laboratories) and housed at, e.g., 5 mice per cage. Food and water are available ad libitum. Mice are acclimated to the animal facilities for a period of at least five days prior to the commencement of experiments. Animals are tested, e.g., in the light phase of a 12-hour light: 12-hour dark schedule (lights on at 06:00 hours). All experiments are conducted in compliance with Ideaya Bioscience's Institutional Animal Care and Use Committee and the NIH Guide for Care and Use of Laboratory Animals Guidelines.

To generate xenografts, a suspension of, e.g., 1 or 3×10⁶ viable tumor cells derived from human colon carcinoma, HCT-116 (ATCC) or HCT-116 shWRN cells are injected subcutaneously into the flank of 6- to 8-week-old mice. The injection volume is, e.g., 0.1 mL, and composed of a 1:1 mixture of HBSS and Matrigel (BD Biosciences). Tumors are size matched at, e.g., approximately 200-250 mm³. Therapy begins the day of or 24 hours after size matching the tumors. Each experimental group includes, e.g., 8-10 animals. Tumors are measured, e.g., two to three times weekly. Measurements of the length (L) and width (W) of the tumor are obtained, e.g., via electronic calipers, and the volume is calculated according to the following equation: V=L×W²/2.

To characterize the knockdown of WRN from the HCT-116 shWRN cells in vivo, animals are administered increasing doses of doxycycline. Doxycycline hyclate is reconstituted, e.g., with 0.9% sodium chloride for injection and administered intraperitoneally at a predetermined interval. Tumors are collected at defined times following administration and WRN knockdown is determined by PCR or Western blots. To determine the effect of sustained WRN knockdown on HCT-116 cells, tumors are size-matched and doxycycline is administered for a predetermined number of days.

Mice are euthanized when tumor volume reaches a maximum of, e.g., 2000 mm³, or upon presentation of skin ulcerations or other morbidities, whichever occur first. For all groups, tumor volumes are plotted only for the duration that allowed the full set of animals to remain on study. If animals have to be taken off study, the remaining animals are monitored for tumor growth until they reach defined endpoints.

Maximal tumor growth inhibition (TGIₘₐₓ), expressed as a percentage, indicates the maximal divergence between the mean tumor volume of the test article-treated group and the control group treated with drug. Tumor growth delay (TGD), expressed as a percentage, is the difference of the median time of the test article-treated group tumors to reach 1 cm³ as compared with the control group. Data from experiments in vivo are analyzed, e.g., using the two-way ANOVA with post hoc Bonferroni correction for TGIₘₐₓ and the Mantel-Cox log-rank test for TGD (e.g., using GraphPad Prism, GraphPad Software).

What is claimed is:

1. A pharmaceutical agent for use in a method for treating a proliferative disease in an individual in need thereof, the proliferative disease being characterized by proliferating cells having a high microsatellite instability (MSI-H), said method comprising administering to the individual said pharmaceutical agent, wherein said pharmaceutical agent is effective for decreasing the helicase activity of WRN in the proliferative cells.

2. The pharmaceutical agent for use according to claim 1, wherein the pharmaceutical agent is an inhibitor of WRN.

3. The pharmaceutical agent for use according to claim 2, wherein the inhibitor of WRN is a small molecule inhibitor.

4. The pharmaceutical agent for use according to claim 3, wherein the small molecule inhibitor has the formula: 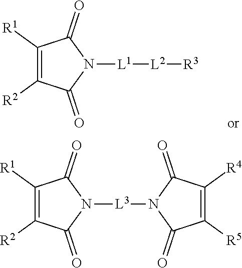
or a pharmaceutically acceptable salt thereof,
wherein
L¹ is C₁₋₄ alkylene;
L² is O, S, OC(O), OSO₂, OC(O)O, or OC(O)NH;
L³ is C₁₋₈ alkylene;
R¹, R², R⁴, and R⁵ are each independently H, halogen, C₁₋₄ alkyl, or C₁₋₄ haloalkyl; and
R³ is H, C₁₋₆ alkyl, C₂₋₆ alkenyl, C₂₋₆ alkynyl, C₁₋₆ hydroxyalkyl, C₁₋₆ haloalkyl, C₆₋₁₂ aryl, or C₆₋₁₂ aryl-C₁₋₄ alkyl, each of which is optionally substituted with halogen, C₁₋₄ alkyl, or C₁₋₄ haloalkyl.

5. The pharmaceutical agent for use according to claim 2, wherein the inhibitor of WRN is an ADC comprising an antibody conjugated to a WRN inhibitor.

6. The pharmaceutical agent for use according to claim 1, wherein the pharmaceutical agent is an inhibitory nucleic acid targeting WRN mRNA, or a nucleic acid encoding the inhibitory nucleic acid.

7. The pharmaceutical agent for use according to claim 6, wherein the inhibitory nucleic acid comprises a short interfering RNA (siRNA), a microRNA (miRNA), or an antisense oligonucleotide.

8. The pharmaceutical agent for use according to claim 1, wherein the pharmaceutical agent is a nuclease capable of modifying the genomes of the proliferative cells such that the helicase activity of WRN in the proliferative cells is decreased, or a nucleic acid encoding the nuclease.

9. The pharmaceutical agent for use according to claim 8, wherein the nuclease is a transcription activator-like effector nuclease (TALEN) or zinc-finger nuclease (ZFN) targeting a genomic sequence within or near an endogenous WRN gene locus.

10. The pharmaceutical agent for use according to claim 8, wherein the method comprises administering to the individual a) a gRNA comprising a spacer sequence that is complementary to a genomic sequence within or near an endogenous WRN gene locus, or a nucleic acid encoding the gRNA; and b) an RNA-guided endonuclease (RGEN), or a nucleic acid encoding the RGEN.

11. The pharmaceutical agent for use according to claim 10, wherein the nucleic acid encoding the gRNA is contained in an Adeno Associated Virus (AAV) vector and/or the nucleic acid encoding the RGEN is contained in an AAV vector.

12. The pharmaceutical agent for use according to claim 10 or 11, wherein the spacer sequence is complementary to a genomic sequence within a coding region of the endogenous WRN gene.

13. The pharmaceutical agent for use according to any one of claims 10-12, wherein the genomes of the proliferative cells are modified by non-homologous end joining (NHEJ).

14. The pharmaceutical agent for use according to any one of claims 10-12, wherein the method further comprises administering to the individual a donor template comprising a donor nucleic acid, wherein the donor template is configured such that the donor nucleic acid is capable of being inserted into the WRN gene locus by homologous recombination.

15. The pharmaceutical agent for use according to claim 14, wherein the donor nucleic acid encodes one or more STOP codons, and the donor template is configured such that the donor nucleic acid is inserted into a coding region of the WRN gene.

16. The pharmaceutical agent for use according to claim 15, wherein the donor nucleic acid encodes three STOP codons in each of the 3 translation frames present in succession.

17. The pharmaceutical agent for use according to claim 14, wherein the donor nucleic acid encodes a mutation in the WRN helicase domain that decreases WRN helicase activity.

18. The pharmaceutical agent for use according to any one of claims 14-17, wherein the donor template is contained in an AAV vector.

19. The pharmaceutical agent for use according to any one of claims 10-18, wherein the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof.

20. The pharmaceutical agent for use according to claim 19, wherein the RGEN is Cas9.

21. The pharmaceutical agent for use according to any one of claims 10-20, wherein the nucleic acid encoding the RGEN is a ribonucleic acid (RNA) sequence.

22. The pharmaceutical agent for use according to claim 21, wherein the RNA sequence encoding the RGEN is linked to the gRNA via a covalent bond.

23. The pharmaceutical agent for use according to any one of claims 10-20, wherein the nucleic acid encoding the RGEN is a deoxyribonucleic acid (DNA) sequence.

24. The pharmaceutical agent for use according to of any one of claims 10-23, wherein the nucleic acid encoding the RGEN is formulated in a liposome or lipid nanoparticle.

25. The pharmaceutical agent for use according to claim 24, wherein the liposome or lipid nanoparticle encapsulates the gRNA.

26. The pharmaceutical agent for use according to any one of claims 10-20, wherein the RGEN is pre-complexed with the gRNA, forming a Ribonucleoprotein (RNP) complex.

27. The pharmaceutical agent for use according to claim 1, wherein the pharmaceutical agent is a nucleic acid construct comprising a donor nucleic acid, wherein the nucleic acid construct is configured such that insertion of the donor nucleic acid into the genomes of the proliferative cells by homologous recombination decreases the helicase activity of WRN in the proliferative cells.

28. The pharmaceutical agent for use according to claim 27, wherein the nucleic acid construct is an AAV vector.

29. The pharmaceutical agent for use according to claim 28, wherein the AAV vector comprises two homology arms having sequences identical or substantially homologous to regions of the endogenous WRN gene.

30. The pharmaceutical agent for use according to claim 28 or 29, wherein the AAV vector is an AAV clade F vector.

31. The pharmaceutical agent for use according to claim 1, where the pharmaceutical agent is a proteolysis targeting chimera (PROTAC) that targets WRN for ubiquitination and proteolytic degradation.

32. The pharmaceutical agent for use according to claim 31, wherein the PROTAC comprises an E3 ubiquitin ligase ligand coupled via a linker to a WRN ligand.

33. The pharmaceutical agent for use according to any one of claims 1-32, wherein the proliferative cells comprise one or more MSI-H markers selected from the group consisting of BAT25, BAT26, D2S123, D5S346, and D17S250.

34. The pharmaceutical agent for use according to claim 33, wherein the method further comprises determining the presence of the one or more MSI-H markers in a population of proliferative cells from the individual to identify the presence of MSI-H in the proliferative cells.

35. The pharmaceutical agent for use according to claim 34, wherein the step of determining the presence of the one or more MSI-H markers is carried out prior to administering the pharmaceutical agent.

36. The pharmaceutical agent for use according to any one of claims 1-35, wherein the proliferative cells comprises a mutation that impairs DNA mismatch repair.

37. The pharmaceutical agent for use according to claim 36, wherein the proliferative cell comprises a mutation in a MutS homolog and/or a mutation in a MutL homolog.

38. The pharmaceutical agent for use according to claim 37, wherein the MutS homolog is selected from the group consisting of MSH2, MSH3, and MSH6, and the MutL homolog is selected from the group consisting of MLH1, MLH3, PMS1, and PMS2.

39. The pharmaceutical agent for use according to claim 38, wherein the proliferative cell comprises a mutation in MLH1, MSH2, and/or PMS2.

40. The pharmaceutical agent for use according to any one of claims 36-39, wherein the method further comprises determining the presence of the mutation in a population of proliferative cells from the individual to identify the presence of the mutation in the proliferative cells.

41. The pharmaceutical agent for use according to claim 40, wherein the step of determining the presence of the mutation is carried out prior to administering the pharmaceutical agent.

42. The pharmaceutical agent for use according to any one of claims 1-41, wherein the proliferative cell comprises one or more markers of DNA damage.

43. The pharmaceutical agent for use according to claim 42, wherein the one or more markers of DNA damage are selected from the group consisting of high p21 expression and high γH2AX expression.

44. The pharmaceutical agent for use according to claim 42 or 43, wherein the method further comprises determining the presence of the one or more markers of DNA damage in a population of proliferative cells from the individual to identify the presence of the one or more markers of DNA damage in the proliferative cells.

45. The pharmaceutical agent for use according to claim 44, wherein the step of determining the presence of the one or more markers of DNA damage is carried out prior to administering the pharmaceutical agent.

46. The pharmaceutical agent for use according to any one of claims 1-45, wherein the amount of proliferative cells in the individual is decreased as compared to a corresponding individual that does not receive administration of the pharmaceutical agent.

47. The pharmaceutical agent for use according to any one of claims 1-46, wherein the rate of proliferation of the proliferative cells is decreased as compared to a corresponding individual that does not receive administration of the pharmaceutical agent.

48. The pharmaceutical agent for use according to any one of claims 1-47, wherein at least some of the proliferative cells are induced to undergo cell cycle arrest.

49. The pharmaceutical agent for use according to any one of claims 1-48, wherein at least some of the proliferative cells are induced to undergo apoptosis.

50. The pharmaceutical agent for use according to any one of claims 1-49, wherein the proliferative disease is a cancer.

51. The pharmaceutical agent for use according to claim 50, wherein the cancer is selected from the group consisting of colon cancer, gastric cancer, endometrium cancer, ovarian cancer, hepatobiliary tract cancer, urinary tract cancer, brain cancer, and skin cancer.

52. The pharmaceutical agent for use according to any one of claims 1-51, wherein the method further comprises administering to the individual a conventional therapy for the proliferative disease.

53. The pharmaceutical agent for use according to claim 52, comprising administering to the individual an anti-PD-1 therapy.

54. The pharmaceutical agent for use according to any one of claims 1-53, wherein the individual is
(a) a mammal;
(b) a human; or
(c) a veterinary animal.

55. A pharmaceutical agent effective for decreasing WRN helicase activity for use in a method of treating an individual with a proliferative disease, the method comprising determining the presence of a high microsatellite instability (MSI-H), or a marker associated with an MSI-H, in a population of proliferative cells from the individual, determining a likelihood that the individual will respond to a therapy comprising administering to the individual said pharmaceutical agent based on the determination of the presence of MSI-H, or a marker associated with MSI-H, in the population of proliferative cells, and administering to the individual said pharmaceutical agent if the individual is predicted to respond to the therapy.

56. The pharmaceutical agent for use according to claim 55, wherein the determination of the presence of MSI-H in the population of proliferative cells comprises determining the presence of one or more MSI markers selected from the group consisting of BAT25, BAT26, D2S123, D5S346, and D17S250.

57. The pharmaceutical agent for use according to claim 55 or 56, wherein the individual is predicted to respond to the therapy if the amount of cells in the population of proliferative cells determined to have at least one of the MSI-H markers is above a pre-determined threshold for the proliferative disease.

58. The pharmaceutical agent for use according to claim 55 or 56, wherein the individual is predicted not to respond to the therapy if
(a) the amount of cells in the population of proliferative cells determined to have at least one of the MSI-H markers is below a pre-determined threshold for the proliferative disease; or
(b) the population of proliferative cells is determined to have none of the MSI-H markers.

59. The pharmaceutical agent for use according to claim 55, wherein the determination of the presence of a marker associated with MSI-H in the population of proliferative cells comprises determining the presence of a mutation that impairs DNA mismatch repair.

60. The pharmaceutical agent for use according to claim 59, wherein the mutation comprises a mutation in a MutS homolog and/or a mutation in a MutL homolog.

61. The pharmaceutical agent for use according to claim 60, wherein the MutS homolog is selected from the group consisting of MSH2, MSH3, and MSH6, and the MutL homolog is selected from the group consisting of MLH1, MLH3, PMS1, and PMS2.

62. The pharmaceutical agent for use according to claim 61, wherein the mutation comprises a mutation in MLH1, MSH2, and/or PMS2.

63. The pharmaceutical agent for use according to claim 55, wherein the determination of the presence of a marker associated with MSI-H in the population of proliferative cells comprises determining the presence of one or more markers of DNA damage.

64. The pharmaceutical agent for use according to claim 63, wherein the one or more markers of DNA damage are selected from the group consisting of high p21 expression and high γH2AX expression.

65. The pharmaceutical agent for use according to any one of claims 59-64, wherein the individual is predicted to respond to the therapy if the amount of cells in the population of proliferative cells determined to have (i) at least one mutation that impairs DNA mismatch repair and/or (ii) at least one marker of DNA damage is above a pre-determined threshold for the proliferative disease.

66. The pharmaceutical agent for use according to claim 65, wherein the at least one mutation that impairs DNA mismatch repair comprises a mutation in MLH1, MSH2, and/or PMS2, and the at least one marker of DNA damage comprises high p21 expression and/or high γH2AX expression.

67. The pharmaceutical agent for use according to any one of claims 59-64, wherein the individual is predicted not to respond to the therapy if
(a) the amount of cells in the population of proliferative cells determined to have (i) at least one mutation that impairs DNA mismatch repair and/or (ii) at least one marker of DNA damage is below a pre-determined threshold for the proliferative disease; or
(b) the population of proliferative cells is determined to have no mutations that impair DNA mismatch repair and no DNA damage markers.

68. An in vitro method for detecting a high microsatellite instability (MSI-H) and the helicase activity of WRN in an individual diagnosed with or thought to have a proliferative disease, the method comprising:
(a) contacting a biological sample from the individual with one or more reagents for detecting the presence of an MSI and the helicase activity of WRN; and
(b) detecting (i) the presence of an MSI-H; and (ii) the helicase activity of WRN.

69. The method of claim 68, wherein the reagent for detecting the presence of an MSI-H in a biological sample comprises a reagent for detecting the presence of one or more MSI markers selected from the group consisting of BAT25, BAT26, D2S123, D5S346, and D17S250.

70. An in vitro method for detecting a marker associated with a high microsatellite instability (MSI-H) and the helicase activity of WRN in an individual diagnosed with or thought to have a proliferative disease, the method comprising:
(a) contacting a biological sample from the individual with one or more reagents for detecting the presence of a marker associated with an MSI-H and the helicase activity of WRN helicase; and
(b) detecting (i) the presence of the marker associated with an MSI-H; and (ii) the helicase activity of WRN helicase.

71. The method of claim 70, wherein the reagent for detecting the presence of a marker associated with an MSI-H in a biological sample comprises a reagent for detecting the presence of (i) one or more mutations that impair DNA mismatch repair and/or (ii) one or more markers of DNA damage.

72. The method of claim 71, wherein the one or more mutations that impair DNA mismatch repair comprise a mutation in a MutS homolog and/or a mutation in a MutL homolog.

73. The method of claim 72, wherein the MutS homolog is selected from the group consisting of MSH2, MSH3, and MSH6, and the MutL homolog is selected from the group consisting of MLH1, MLH3, PMS1, and PMS2.

74. The method of claim 73, wherein the one or more mutations comprise a mutation in MLH1, MSH2, and/or PMS2.

75. The method of any one of claims 71-74, wherein the one or more markers of DNA damage are selected from the group consisting of high p21 expression and high γH2AX expression.

76. The method of any one of claims 55-75, wherein the proliferative disease is a cancer.

77. The method of claim 76, wherein the cancer is selected from the group consisting of colon cancer, gastric cancer, endometrium cancer, ovarian cancer, hepatobiliary tract cancer, urinary tract cancer, brain cancer, and skin cancer.

78. The method of any one of claims 55-77, wherein the individual is
(a) a mammal;
(b) a human; or
(c) a veterinary animal.

79. A composition comprising (a) a gRNA comprising a spacer sequence complementary to a genomic sequence within or near an endogenous WRN gene locus, or a nucleic acid encoding the gRNA; and b) an RNA-guided endonuclease (RGEN), or a nucleic acid encoding the RGEN, wherein the components of the composition are configured such that delivery of the composition into a cell is capable of decreasing the helicase activity of WRN in the cell.

80. The composition of claim 79, wherein the nucleic acid encoding the gRNA is contained in an Adeno Associated Virus (AAV) vector and/or the nucleic acid encoding the RGEN is contained in an AAV vector.

81. The composition of claim 79 or 80, wherein the spacer sequence is complementary to a genomic sequence within a coding region of the endogenous WRN gene.

82. The composition of any one of claims 79-81, further comprising a donor template comprising a donor nucleic acid, wherein the donor template is configured such that the donor nucleic acid is capable of being inserted into the WRN gene locus by homologous recombination.

83. The composition of claim 82, wherein the donor nucleic acid encodes one or more STOP codons, and the donor template is configured such that the donor nucleic acid is inserted into a coding region of the WRN gene.

84. The composition of claim 83, wherein the donor nucleic acid encodes three STOP codons in each of the 3 translation frames present in succession.

85. The composition of claim 82, wherein the donor nucleic acid encodes a mutation in the WRN helicase domain that decreases WRN helicase activity.

86. The composition of any one of claims 82-85, wherein the donor template is contained in an AAV vector.

87. The composition of any one of claims 79-86, wherein the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof.

88. The composition of claim 87, wherein the RGEN is Cas9.

89. The composition of any one of claims 79-88, wherein the nucleic acid encoding the RGEN is a ribonucleic acid (RNA) sequence.

90. The composition of claim 89, wherein the RNA sequence encoding the RGEN is linked to the gRNA via a covalent bond.

91. The composition of any one of claims 79-88, wherein the nucleic acid encoding the RGEN is a deoxyribonucleic acid (DNA) sequence.

92. The composition of any one of claims 79-91, wherein the nucleic acid encoding the RGEN is formulated in a liposome or lipid nanoparticle.

93. The composition of claim 92, wherein the liposome or lipid nanoparticle encapsulates the gRNA.

94. The composition of any one of claims 79-88, wherein the RGEN is pre-complexed with the gRNA, forming a Ribonucleoprotein (RNP) complex.

95. A composition comprising a nucleic acid construct comprising a donor nucleic acid, wherein the nucleic acid construct is configured such that insertion of the donor nucleic acid into the genome of a proliferative cell by homologous recombination decreases the helicase activity of WRN in the proliferative cell.

96. The composition of claim 95, wherein the nucleic acid construct is an AAV vector.

97. The composition of claim 96, wherein the AAV vector comprises two homology arms having sequences identical or substantially homologous to regions of the endogenous WRN gene.

98. The composition of any one of claims 95-97, wherein the AAV vector is an AAV clade F vector.

99. A method for decreasing proliferation in a proliferative cell having a high microsatellite instability (MSI-H), comprising decreasing the helicase activity of Werner syndrome ATP-dependent helicase (WRN) in the proliferative cell.

100. The method of claim 99, comprising delivering into the proliferative cell an inhibitor of WRN.

101. The method of claim 100, wherein the inhibitor of WRN is a small molecule inhibitor.

102. The method of claim 101, wherein the small molecule inhibitor has the formula: 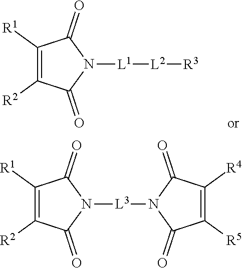
or a pharmaceutically acceptable salt thereof,
wherein
L¹ is C₁₋₄ alkylene;
L² is O, S, OC(O), OSO₂, OC(O)O, or OC(O)NH;
L³ is C₁₋₈ alkylene;
R¹, R², R⁴ and R⁵ are each independently H, halogen, C₁₋₄ alkyl, or C₁₋₄ haloalkyl; and
R³ is H, C₁₋₆ alkyl, C₂₋₆ alkenyl, C₂₋₆ alkynyl, C₁₋₆ hydroxyalkyl, C₁₋₆ haloalkyl, C₆₋₁₂ aryl, or C₆₋₁₂ aryl-C₁₋₄ alkyl, each of which is optionally substituted with halogen, C₁₋₄ alkyl, or C₁₋₄ haloalkyl.

103. The method of claim 100, wherein the inhibitor of WRN comprises an antibody drug conjugate (ADC) comprising an antibody conjugated to the WRN inhibitor.

104. The method of claim 99, comprising delivering into the proliferative cell an inhibitory nucleic acid targeting WRN mRNA, or a nucleic acid encoding the inhibitory nucleic acid.

105. The method of claim 104, wherein the inhibitory nucleic acid comprises a short interfering RNA (siRNA), a microRNA (miRNA), or an antisense oligonucleotide.

106. The method of claim 99, comprising delivering into the proliferative cell a nuclease capable of modifying the genome of the proliferative cell such that the helicase activity of WRN in the proliferative cell is decreased, or a nucleic acid encoding the nuclease.

107. The method of claim 106, wherein the nuclease is a transcription activator-like effector nuclease (TALEN) or zinc-finger nucleases (ZFN) targeting a genomic sequence within or near an endogenous WRN gene locus.

108. The method of claim 106, comprising delivering into the proliferative cell a) a gRNA comprising a spacer sequence that is complementary to a genomic sequence within or near an endogenous WRN gene locus, or a nucleic acid encoding the gRNA; and b) an RNA-guided endonuclease (RGEN), or a nucleic acid encoding the RGEN.

109. The method of claim 108, wherein the nucleic acid encoding the gRNA is contained in an Adeno Associated Virus (AAV) vector and/or the nucleic acid encoding the RGEN is contained in an AAV vector.

110. The method of claim 108 or 109, wherein the spacer sequence is complementary to a genomic sequence within a coding region of the endogenous WRN gene.

111. The method of any one of claims 108-110, wherein the genome of the proliferative cell is modified by non-homologous end joining (NHEJ).

112. The method of any one of claims 108-110, further comprising delivering into the proliferative cell a donor template comprising a donor nucleic acid, wherein the donor template is configured such that the donor nucleic acid is capable of being inserted into the WRN gene locus by homologous recombination.

113. The method of claim 112, wherein the donor nucleic acid encodes one or more STOP codons, and the donor template is configured such that the donor nucleic acid is inserted into a coding region of the WRN gene.

114. The method of claim 113, wherein the donor nucleic acid encodes three STOP codons in each of the 3 translation frames present in succession.

115. The method of claim 112, wherein the donor nucleic acid encodes a mutation in the WRN helicase domain that decreases WRN helicase activity.

116. The method of any one of claims 112-115, wherein the donor template is contained in an AAV vector.

117. The method of any one of claims 108-116, wherein the RGEN is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof.

118. The method of claim 17, wherein the RGEN is Cas9.

119. The method of any one of claims 108-118, wherein the nucleic acid encoding the RGEN is a ribonucleic acid (RNA) sequence.

120. The method of claim 119, wherein the RNA sequence encoding the RGEN is linked to the gRNA via a covalent bond.

121. The method of any one of claims 108-118, wherein the nucleic acid encoding the RGEN is a deoxyribonucleic acid (DNA) sequence.

122. The method of any one of claims 108-121, wherein the nucleic acid encoding the RGEN is formulated in a liposome or lipid nanoparticle.

123. The method of claim 122, wherein the liposome or lipid nanoparticle encapsulates the gRNA.

124. The method of any one of claims 108-118, wherein the RGEN is pre-complexed with the gRNA, forming a Ribonucleoprotein (RNP) complex.

125. The method of claim 99, comprising delivering into the proliferative cell a nucleic acid construct comprising a donor nucleic acid, wherein the nucleic acid construct is configured such that insertion of the donor nucleic acid into the genome of the proliferative cell by homologous recombination decreases the helicase activity of WRN in the proliferative cell.

126. The method of claim 125, wherein the nucleic acid construct is an AAV vector.

127. The method of claim 126, wherein the AAV vector comprises two homology arms having sequences identical or substantially homologous to regions of the endogenous WRN gene.

128. The method of claim 126 or 127, wherein the AAV vector is an AAV clade F vector.

129. The method of claim 99, comprising delivering into the proliferative cell a PROTAC that targets WRN for ubiquitination and proteolytic degradation.

130. The method of claim 129, wherein the PROTAC comprises an E3 ubiquitin ligase ligand coupled via a linker to a WRN ligand.

131. The method of any one of claims 99-130, wherein the proliferative cell comprises one or more MSI markers selected from the group consisting of BAT25, BAT26, D2S123, D5S346, and D17S250.

132. The method of any one of claims 99-131, wherein the proliferative cell comprises a mutation that impairs DNA mismatch repair.

133. The method of claim 132, wherein the proliferative cell comprises a mutation in a MutS homolog and/or a mutation in a MutL homolog.

134. The method of claim 133, wherein the MutS homolog is selected from the group consisting of MSH2, MSH3, and MSH6, and the MutL homolog is selected from the group consisting of MLH1, MLH3, PMS1, and PMS2.

135. The method of claim 134, wherein the proliferative cell comprises a mutation in MLH1, MSH2, and/or PMS2.

136. The method of any one of claims 99-135, wherein the proliferative cell comprises one or more markers of DNA damage.

137. The method of claim 136, wherein the one or more markers of DNA damage are selected from the group consisting of high p21 expression and high γH2AX expression.

138. The method of any one of claims 99-137, wherein decreasing proliferation in the proliferative cell comprises inducing cell cycle arrest in the proliferative cell.

139. The method of any one of claims 99-137, wherein decreasing proliferation in the proliferative cell comprises inducing apoptosis in the proliferative cell.

140. The method of any one of claims 99-139, wherein the method is carried out in vivo.

141. The method of any one of claims 99-139, wherein the method is carried out ex vivo.

142. The method of any one of claims 99-139, wherein the method is carried out in vitro.

143. The method of any one of claims 99-142, wherein the proliferative cell is a cancer cell.

144. The method of claim 143, wherein the cancer is selected from the group consisting of colon cancer, gastric cancer, endometrium cancer, ovarian cancer, hepatobiliary tract cancer, urinary tract cancer, brain cancer, and skin cancer.

145. The method of any one of claims 99-144, wherein the proliferative cell is
(a) a mammalian cell;
(b) a human cell; or
(c) a veterinary animal cell.


SEQUENCE LISTING (16 sequences)

SEQ ID NO: 1 - DNA, 21 bp, Artificial sequence
  Synthetic polynucleotide
  misc_feature: siRNA s14907, WRN gene
aguaagauagaaacccuccgt

SEQ ID NO: 2 - DNA, 25 bp, Artificial sequence
  Synthetic polynucleotide
  misc_feature: siRNA NM_000553_stealth_691, WRN 5′UTR
aaacccgagaagauccaguccaaca

SEQ ID NO: 3 - DNA, 21 bp, Artificial sequence
  Synthetic polynucleotide
  misc_feature: siRNA s1999, BLM gene
uuucguuuuggaagauauctt

SEQ ID NO: 4 - DNA, 21 bp, Artificial sequence
  Synthetic polynucleotide
  misc_feature: siRNA s297, MLH 1.1 gene
auauuguccacgguugaggca

SEQ ID NO: 5 - DNA, 21 bp, Artificial sequence
  Synthetic polynucleotide
  misc_feature: siRNA s298, MLH 1.2 gene
uauuguccacgguugaggcat

SEQ ID NO: 6 - DNA, 21 bp, Artificial sequence
  Synthetic polynucleotide
  misc_feature: siRNA s224048, MLH 1.3 gene
uauccucacauccaauuucta

SEQ ID NO: 7 - DNA, 21 bp, Artificial sequence
  Synthetic polynucleotide
  misc_feature: siRNA s8966, MSH2 1.1 gene
uuacacgaaaguaauauccaa

SEQ ID NO: 8 - DNA, 21 bp, Artificial sequence
  Synthetic polynucleotide
  misc_feature: siRNA s8967, MSH2 1.2 gene
uaagaucugggaaucgacgaa

SEQ ID NO: 9 - DNA, 21 bp, Artificial sequence
  Synthetic polynucleotide
  misc_feature: siRNA s8968, MSH2 1.3 gene
uaucauauccuugcgauuctc

SEQ ID NO: 10 - DNA, 21 bp, Artificial sequence
  Synthetic polynucleotide
  misc_feature: siRNA s229950, PMS1 1.1 gene
ucuacauucauaaacuucctt

SEQ ID NO: 11 - DNA, 21 bp, Artificial sequence
  Synthetic polynucleotide
  misc_feature: siRNA s229951, PMS1 1.2 gene
acaaguuuuacuauauuccgt

SEQ ID NO: 12 - DNA, 21 bp, Artificial sequence
  Synthetic polynucleotide
  misc_feature: siRNA s229952, PMS1 1.3 gene
ucuuuuaaaucugcuacucca

SEQ ID NO: 13 - DNA, 21 bp, Artificial sequence
  Synthetic polynucleotide
  misc_feature: siRNA s10740, PMS2 1.1 gene
auuggugcaacuuacacggat

SEQ ID NO: 14 - DNA, 21 bp, Artificial sequence
  Synthetic polynucleotide
  misc_feature: siRNA s10742, PMS2 1.2 gene
aaacucgaaauuuacauccgg

SEQ ID NO: 15 - DNA, 21 bp, Artificial sequence
  Synthetic polynucleotide
  misc_feature: siRNA s534928, PMS2 1.3 gene
ucuuguagcaaaauuugcctt

SEQ ID NO: 16 - DNA, 21 bp, Artificial sequence
  Synthetic polynucleotide
  misc_feature: siRNA s7903, Kifl 1 gene
ugaacuuagaagaucaguctt